SEQUENCE LISTING (83 sequences)

SEQ ID NO: 1 - DNA, 1110 bp, Neisseria meningitidis (group B)
atggatgtcagcctgtacggcgaaatcaaagccggcgtggaaggcaggaacatccagctgcagttgaccgaaccgctcccaaatattcaacctcaggttactaagcgcaaaagccgcatcaggacgaaaatcagcgatttcggctcgtttatcggctttaaggggagtgaggatttgggcgaagggctgaaggctgtttggcagcttgagcaagacgtatccgttgccggcggcggcgcgtcccagtggggcaacagggaatcctttatcggcttggcaggcgaattcggtacgctgcgcgccggtcgcgttgcaaatcagtttgacgatgccagccaagccattgatccttgggacagcaataatgatgtggcttcgcaattgggtattttcaaacgccacgacgatatgtcggtttccgtacgctacgattcccccgaattttccggttttagcggcagcgtccaattcgttccggcccaaaacagcaagtccgcctatacgccggctcattttgttcagaataagcaaaatcagcggcctactctcgttccggctgttgtcggcaagccggggtcggatgtgtattatgccggtctgaattacaaaaatggcggttttgccgggaactatgcctttaaatacgcgaaacacgccaatgtgggccgtgatgcttttgagttgttcttgatcggcagcgcgacgagtgatgaagccaaaggtaccgatcccttgaaaaaccatcaggtacaccgcctgacgggcggctatgaggaaggcggcttgaatctcgccttggcggcccagttggatttgtctgaaaatggcgacaaagccaaaaccaaaaacagtacgaccgaaattgccgcgactgcttcctaccgcttcggtaatgcagttccacgcatcagctatgcccatggtttcgacttgatcgaacgcggtaaaaaaggcgaaaataccagctacgatcaaatcatcgccggcgttgattatgatttttccaaacgcacttccgccatcgtgtctggcgcttggctgaaacgcaataccggcatcggcaactacactcaaattaatgccgcctccgtcggtttgcgccacaaattc

SEQ ID NO: 2 - protein, 1057 aa, Neisseria meningitidis (group B)
METASPVALSERLETYRGLYGLILELYSALAGLYVALGLGLYARGASNILEGLNLEGLNLETHRGLPRLEPRASNILEGLNPRGLNVALTHRLYSARGLYSSERARGILEARGTHRLYSILESERASPPHEGLYSERPHEILEGLYPHELYSGLYSERGLASPLEGLYGLGLYLELYSALAVALTRPGLNLEGLGLNASPVALSERVALALAGLYGLYGLYALASERGLNTRPGLYASNARGGLSERPHEILEGLYLEALAGLYGLPHEGLYTHRLEARGALAGLYARGVALALAASNGLNPHEASPASPALASERGLNALAILEASPPRTRPASPSERASNASNASPVALALASERGLNLEGLYILEPHELYSARGHISASPASPMETSERVALSERVALARGTYRASPSERPRGLPHESERGLYPHESERGLYSERVALGLNPHEVALPRALAGLNASNSERLYSSERALATYRTHRPRALAHISPHEVALGLNASNLYSGLNASNGLNARGPRTHRLEVALPRALAVALVALGLYLYSPRGLYSERASPVALTYRTYRALAGLYLEASNTYRLYSASNGLYGLYPHEALAGLYASNTYRALAPHELYSTYRALALYSHISALAASNVALGLYARGASPALAPHEGLLEPHELEILEGLYSERALATHRSERASPGLALALYSGLYTHRASPPRLELYSASNHISGLNVALHISARGLETHRGLYGLYTYRGLGLGLYGLYLEASNLEALALEALAALAGLNLEASPLESERGLASNGLYASPLYSALALYSTHRLYSASNSERTHRTHRGLILEALAALATHRALASERTYRARGPHEGLYASNALAVALPRARGILESERTYRALAHISGLYPHEASPLEILEGLARGGLYLYSLYSGLYGLASNTHRSERTYRASPGLNILEILEALAGLYVALASPTYRASPPHESERLYSARGTHRSERALAILEVALSERGLYALATRPLELYSARGASNTHRGLYILEGLYASNTYRTHRGLNILEASNALAALASERVALGLYLEARGHISLYSPHE

SEQ ID NO: 3 - DNA, 1104 bp, Neisseria meningitidis (group B)
atggatgttagcctgtacggcgaaatcaaagccggcgtggaaggcaggaacatccagctgcagttgaccgaaccgctccaaaatattcaacaacctcaggttactaagcgcaaaagccgcatcaggacgaaaatcagcgatttcggctcgtttatcggctttaaggggagcgaggatttgggcgaagggctgaaggctgtttggcagcttgagcaagacgtatccgttgccggcggcggcgcgacccgttggggcaacagggaatcctttgtcggcttggcaggtgaattcggcacgctgcgcgccggccgcgttgcgaatcagtttgacgatgccagcaaagccattgatccttgggacagcaataatgttgtggcttcgcaattgggtattttcaaacgccacgacgatatgccggtttccgtacgctacgattccccggaattttccggtttcagcggcagcgtccaattcgttccggctcaaaacagcaagtccgcctatacgccggctcattttgttcagcagactcctcaaagtcagcctactctcgttccggctgttgtcggcaagccggggtcggatgtgtattatgccggtctgaattacaaaaatggcggttttgccgggaactatgcctttaaatacgcgaaacacgccaatgtgggccgtgatgcttttgagttgttcttgctcggcagcgggagtgatgaagccaaaggtaccgatcccttgaaaaaccatcaggtacaccgcctgacgggcggctatgaggaaggcggcttgaatctcgccttggcggctcagttggatttgtctgaaaatgccgacaaaaccaaaaacagtacgaccgaaattgccgccactgcttcctaccgcttcggtaatgcagttccacgcatcagctatgcccatggtttcgactttatcgaacgcggtaaaaaaggcgaaaataccagctacgatcaaatcatcgccggcgttgattatgatttttccaaacgcacttccgccatcgtgtctggcgcttggctgaaacgcaataccggcatcggcaactacactcaaattaatgccgcctccgtcggtttgcgccacaaattc

SEQ ID NO: 4 - protein, 1050 aa, Neisseria meningitidis (group B)
METASPVALSERLETYRGLYGLILELYSALAGLYVALGLGLYARGASNILEGLNLEGLNLETHRGLPRLEGLNASNILEGLNGLNPRGLNVALTHRLYSARGLYSSERARGILEARGTHRLYSILESERASPPHEGLYSERPHEILEGLYPHELYSGLYSERGLASPLEGLYGLGLYLELYSALAVALTRPGLNLEGLGLNASPVALSERVALALAGLYGLYGLYALATHRARGTRPGLYASNARGGLSERPHEVALGLYLEALAGLYGLPHEGLYTHRLEARGALAGLYARGVALALAASNGLNPHEASPASPALASERLYSALAILEASPPRTRPASPSERASNASNVALVALALASERGLNLEGLYILEPHELYSARGHISASPASPMETPRVALSERVALARGTYRASPSERPRGLPHESERGLYPHESERGLYSERVALGLNPHEVALPRALAGLNASNSERLYSSERALATYRTHRPRALAHISPHEVALGLNGLNTHRPRGLNSERGLNPRTHRLEVALPRALAVALVALGLYLYSPRGLYSERASPVALTYRTYRALAGLYLEASNTYRLYSASNGLYGLYPHEALAGLYASNTYRALAPHELYSTYRALALYSHISALAASNVALGLYARGASPALAPHEGLLEPHELELEGLYSERGLYSERASPGLALALYSGLYTHRASPPRLELYSASNHISGLNVALHISARGLETHRGLYGLYTYRGLGLGLYGLYLEASNLEALALEALAALAGLNLEASPLESERGLASNALAASPLYSTHRLYSASNSERTHRTHRGLILEALAALATHRALASERTYRARGPHEGLYASNALAVALPRARGILESERTYRALAHISGLYPHEASPPHEILEGLARGGLYLYSLYSGLYGLASNTHRSERTYRASPGLNILEILEALAGLYVALASPTYRASPPHESERLYSARGTHRSERALAILEVALSERGLYALATRPLELYSARGASNTHRGLYILEGLYASNTYRTHRGLNILEASNALAALASERVALGLYLEARGHISLYSPHE

SEQ ID NO: 5 - DNA, 1122 bp, Neisseria meningitidis (group B)
atggatgtcagcctatacggcgaaatcaaagccggcgtggaaggcaggaactaccagctgcaattgactgaagcacaagccgctaacggtggagcgagcggtcaggtaaaagttactaaagttactaaggccaaaagccgcatcaggacgaaaatcagtgatttcggctcgtttatcggctttaaggggagtgaggatttgggcgacgggctgaaggctgtttggcagcttgagcaagacgtatccgttgccggcggcggcgcgacccagtggggcaacagggaatcctttatcggcttggcaggcgaattcggtacgctgcgcgccggtcgcgttgcgaatcagtttgacgatgccagccaagccattgatccttgggacagcaataatgatgtggcttcgcaattgggtattttcaaacgccacgacgacatgccggtttccgtacgctacgattcccccgaattttccggtttcagcggcagcgttcaattcgttccgatccaaaacagcaagtccgcctatacgccggcttattatactaaggatacaaacaataatcttactctcgttccggctgttgtcggcaagcccggatcggatgtgtattatgccggtctgaattacaaaaatggcggttttgccgggaactatgcctttaaatatgcgagacacgccaatgtcggacgtaatgcttttgagttgttcttgatcggcagcgggagtgatcaagccaaaggtaccgatcccttgaaaaaccatcaggtacaccgtctgacgggcggctatgaggaaggcggcttgaatctcgccttggcggctcagttggatttgtctgaaaatggcgacaaaaccaaaaacagtacgaccgaaattgccgccactgcttcctaccgcttcggtaatgcagttccacgcatcagctatgcccatggtttcgactttatcgaacgcggtaaaaaaggcgaaaataccagctacgatcaaatcatcgccggcgttgattatgatttttccaaacgcacttccgccatcgtgtctggcgcttggctgaaacgcaataccggcatcggcaactacactcaaattaatgccgcctccgtcggtttgcgccacaaattc

SEQ ID NO: 6 - protein, 374 aa, Neisseria meningitidis (group B)
MDVSLYGEIKAGVEGRNYQLQLTEAQAANGGASGQVKVTKVTKAKSRIRTKISDFGSFIGFKGSEDLGDGLKAVWQLEQDVSVAGGGATQWGNRESFIGLAGEFGTLRAGRVANQFDDASQAIDPWDSNNDVASQLGIFKRHDDMPVSVRYDSPEFSGFSGSVQFVPIQNSKSAYTPAYYTKDTNNNLTLVPAVVGKPGSDVYYAGLNYKNGGFAGNYAFKYARHANVGRNAFELFLIGSGSDQAKGTDPLKNHQVHRLTGGYEEGGLNLALAAQLDLSENGDKTKNSTTEIAATASYRFGNAVPRISYAHGFDFIERGKKGENTSYDQIIAGVDYDFSKRTSAIVSGAWLKRNTGIGNYTQINAASVGLRHKF

SEQ ID NO: 7 - DNA, 1113 bp, Neisseria meningitidis (group B)
atggatgtcagcctatacggcgaaatcaaagccggcgtggaaggcaggaactaccagctgcaattgactgaagcacaagccgctaacggtggagcgagcggtcaggtaaaagttactaaggccaaaagccgcatcaggacgaaaatcagtgatttcggctcgtttatcggctttaaggggagcgaggatttgggcgaaggtctgaaagctgtttggcagcttgagcaagacgtatccgttgccggcggcggcgcgacccagtggggcaacagggaatcctttatcggcttggcaggcgaattcggtacgctgcgcgccggtcgcgttgcgaatcagtttgacgatgccagccaagccattgatccttgggacagcaacaatgatgtggcttcgcaattgggtattttcaaacgccacgacgatatgccggtttctgtacgctacgactctccggacttttccggtttcagcggcagcgttcaattcgttccgatccaaaacagcaagtccgcctatacgccggctcatgttgttgtgaataacaaggttgctactcacgttccggctgttgtcggcaagcccggatcggatgtgtattatgccggtctgaattacaaaaatggcggttttgccgggaactatgcctttaaatatgcgagacacgccaatgtcggacgtaatgcttttgagttgttcttgatcggcagcgcgacgagtgatcaagccaaaggtaccgatcccttgaaaaaccatcaggtacaccgcctgacgggcggctatgaggaaggcggcttgaatctcgccttggcggctcagttggatttgtctgaaaatgccgacaaaaccaaaaacagtacgaccgaaattgccgcgactgcttcctaccgcttcggtaatgcagttccacgcatcagctatgcccatggtttcgacttgatcgaacgcggtaaaaaaggcgaaaataccagctacgatcaaatcatcgccggcgttgattatgatttttccaaacgcacttccgccatcgtgtctggcgcttggctgaaacgcaataccggcatcggcaactacactcaaattaatgccgcctccgtcggtttgcgccacaaattc

SEQ ID NO: 8 - protein, 371 aa, Neisseria meningitidis (group B)
MDVSLYGEIKAGVEGRNYQLQLTEAQAANGGASGQVKVTKAKSRIRTKISDFGSFIGFKGSEDLGEGLKAVWQLEQDVSVAGGGATQWGNRESFIGLAGEFGTLRAGRVANQFDDASQAIDPWDSNNDVASQLGIFKRHDDMPVSVRYDSPDFSGFSGSVQFVPIQNSKSAYTPAHVVVNNKVATHVPAVVGKPGSDVYYAGLNYKNGGFAGNYAFKYARHANVGRNAFELFLIGSATSDQAKGTDPLKNHQVHRLTGGYEEGGLNLALAAQLDLSENADKTKNSTTEIAATASYRFGNAVPRISYAHGFDLIERGKKGENTSYDQIIAGVDYDFSKRTSAIVSGAWLKRNTGIGNYTQINAASVGLRHKF

SEQ ID NO: 9 - DNA, 1107 bp, Neisseria meningitidis (group B)
atggatgtcagcctgtacggcgaaatcaaagccggcgtggaaggcaggaacttccagctgcagttgaccgaaccgccctcaaagagtcaacctcaggtaaaagttactaaggccaaaagccgcatcaggacgaaaatcagtgatttcggctcgtttatcggctttaaggggagtgaggatttgggcgaagggctgaaggctgtttggcagcttgagcaagacgtatccgttgccggcggcggcgcgacccagtggggcaacagggaatcctttgtcggcttggcaggcgaattcggtacgctgcgcgccggtcgcgttgcgaatcagtttgacgatgccagccaagccattgatccttgggacagcaacaatgatgtggcttcgcaattgggtattttcaaacgccacgacgatatgccggtttccgtacgctacgactctccggacttttccggtttcagcggcagcgtccaattcgttccgatccaaaacagcaagtccgcctatacgccggctcattatactaggcagaacaatgctgatgttttcgttccggctgttgtcggcaagcccggatcggatgtgtattatgccggtctgaattacaaaaatggcggttttgccgggagctatgcctttaaatatgcgagacacgccaatgtcggacgtgatgcttttgagttgttcttgctcggcagcacgagtgatgaagccaaaggtaccgatcccttgaaaaaccatcaggtacaccgcctgacgggcggctatgaggaaggcggcttgaatctcgccttggcggctcagttggatttgtctgaaaatggcgacaaagccaaaaccaaaaacagtacgaccgaaattgccgccactgcttcctaccgcttcggtaatgcagttccgcgcatcagctatgcccatggtttcgacttgatcgaacgcggtaaaaaaggcgaaaataccagctacgatcaaatcatcgccggcgttgattatgatttttccaaacgcacttccgccatcgtgtctggcgcttggctgaaacgcaataccggcatcggcaactacactcaaattaatgccgcctccgtcggtttgcgccacaaattc

SEQ ID NO: 10 - protein, 369 aa, Neisseria meningitidis (group B)
MDVSLYGEIKAGVEGRNFQLQLTEPPSKSQPQVKVTKAKSRIRTKISDFGSFIGFKGSEDLGEGLKAVWQLEQDVSVAGGGATQWGNRESFVGLAGEFGTLRAGRVANQFDDASQAIDPWDSNNDVASQLGIFKRHDDMPVSVRYDSPDFSGFSGSVQFVPIQNSKSAYTPAHYTRQNNADVFVPAVVGKPGSDVYYAGLNYKNGGFAGSYAFKYARHANVGRDAFELFLLGSTSDEAKGTDPLKNHQVHRLTGGYEEGGLNLALAAQLDLSENGDKAKTKNSTTEIAATASYRFGNAVPRISYAHGFDLIERGKKGENTSYDQIIAGVDYDFSKRTSAIVSGAWLKRNTGIGNYTQINAASVGLRHKF

SEQ ID NO: 11 - DNA, 1110 bp, Neisseria meningitidis (group B)
atggatgtcagcctgtacggcgaaatcaaagccggcgtggaaggcaggaactaccagctgcaattgactgaacaaccctcaagaactcaaggtcaaacgagcaatcaggtaaaagttactaaggccaaaagccgcatcaggacgaaaatcagtgatttcggctcgtttatcggctttaaggggagcgaggatttgggcgaaggtctgaaggctgtttggcagcttgagcaagacgtatccgttgccggcggcggcgcgacccgttggggcaacagggaatcctttgtcggcttggcaggcgaattcggcacgctgcgcgccggccgcgttgcgaatcagtttgacgatgccagcaaagccattgatccttgggacagcaataatgttgtggcttcgcaattgggtattttcaaacgccacgacgatatgccggtttccgtacgctacgactctccggacttttccggtttcagcggcagcgtccaattcgttccggctcaaaacagcaagtccgcctataagccggcttatgtggatgagaagaaaatggttcatgcggctgttgtcggcaagcccggatcggatgtgtattatgccggtctgaattacaaaaatggcggttttgccgggaactatgcctttaaatatgcgaaacacgccaatgtgggccgtgatgcttttaatttgttcttgcttgggcgcatcggcgatgatgatgaagccaaaggtaccgatcccttgaaaaaccatcaggtacaccgcctgacgggcggctatgaggaaggcggcttgaatctcgccttggcggctcagttggatttgtctgaaaatggcgacaaaaccaaaaacagtacgaccgaaattgccgccactgcttcctaccgcttcgggaatgcagttccacgcatcagctatgcccatggtttcgactttatcgaacgcggtaaaaaaggcgaacataccagctacgatcaaatcatcgccggcgttgattatgatttttccaaacgcacttccgccatcgtgtctggtgcttggctgaaacgcaataccggcatcggcaactacactcaaattaatgccgcctccgtcggtttgcgccacaaattc

SEQ ID NO: 12 - protein, 370 aa, Neisseria meningitidis (group B)
MDVSLYGEIKAGVEGRNYQLQLTEQPSRTQGQTSNQVKVTKAKSRIRTKISDFGSFIGFKGSEDLGEGLKAVWQLEQDVSVAGGGATRWGNRESFVGLAGEFGTLRAGRVANQFDDASKAIDPWDSNNVVASQLGIFKRHDDMPVSVRYDSPDFSGFSGSVQFVPAQNSKSAYKPAYVDEKKMVHAAVVGKPGSDVYYAGLNYKNGGFAGNYAFKYAKHANVGRDAFNLFLLGRIGDDDEAKGTDPLKNHQVHRLTGGYEEGGLNLALAAQLDLSENGDKTKNSTTEIAATASYRFGNAVPRISYAHGFDFIERGKKGEHTSYDQIIAGVDYDFSKRTSAIVSGAWLKRNTGIGNYTQINAASVGLRHKF

SEQ ID NO: 13 - DNA, 1104 bp, Neisseria meningitidis (group B)
atggatgtcagcctgtacggcgaaatcaaagccggcgtggaaggcaggaacatccagctgcaattgaccgagcagccctcaaaggctcaaggtcaaacgaacaatcaggtaaaagttactaaggcaaaaagccgcatcaggacgaaaatcagcgatttcggctcgtttatcggctttaaggggagcgaggatttgggcgaagggctgaaggctgtttggcagcttgagcaagacgtatccgttgccggcggcggcgcgacccagtggggcaacagggaatcctttgtcggcttggcaggcgaattcggtacgctgcgcgccggtcgcgttgcaaatcagtttgacgatgccagccaagccattgatccttgggacagcaacaatgatgtggcttcgcaattgggtattttcaaacgccacgacgatatgccggtttccgtacgctacgactctccggacttttccggtttcagcggcagcgtccaattcgttccggctcaaaacagcaagtccgcctatacgccggcttatgtggatgagcagagtaagtatcatgcggctgttgtcggcaagcccggatcggatgtgtattatgccggtctgaattataagaatggcggttttgccgggaactatgcctttaaatatgcgaaacacgccaatgtcggacgtgatgcttttgagttgttcttgctcggcagcgggagtgatcaagccaaaggtaccgatcccttgaaaaaccatcaggtacaccgcctgacgggcggctatgaggaaggcggcttgaatctcgccttggcggctcagttggatttgtctgaaaatgccgacaaaaccaaaaacagtacgaccgaaattgccgccactgcttcctaccgcttcggtaatgcagttccacgcatcagctatgcccatggtttcgacttgatcgaacgcggtaaaaaaggcgaaaataccagctacgatcaaatcatcgccggcgttgattatgatttttccaaacgcacttccgccatcgtgtctggcgcttggctgaaacgcaataccggcatcggcaactacactcaaattaatgccgcctccgtcggtttgcgccacaaattc

SEQ ID NO: 14 - protein, 1056 aa, Neisseria meningitidis (group B)
METASPVALSERLETYRGLYGLILELYSALAGLYVALGLGLYARGASNILEGLNLEGLNLETHRGLGLNPRSERLYSALAGLNGLYGLNTHRASNASNGLNVALLYSVALTHRLYSALALYSSERARGILEARGTHRLYSILESERASPPHEGLYSERPHEILEGLYPHELYSGLYSERGLASPLEGLYGLGLYLELYSALAVALTRPGLNLEGLGLNASPVALSERVALALAGLYGLYGLYALATHRGLNTRPGLYASNARGGLSERPHEVALGLYLEALAGLYGLPHEGLYTHRLEARGALAGLYARGVALALAASNGLNPHEASPASPALASERGLNALAILEASPPRTRPASPSERASNASNASPVALALASERGLNLEGLYILEPHELYSARGHISASPASPMETPRVALSERVALARGTYRASPSERPRASPPHESERGLYPHESERGLYSERVALGLNPHEVALPRALAGLNASNSERLYSSERALATYRTHRPRALATYRVALASPGLGLNSERLYSTYRHISALAALAVALVALGLYLYSPRGLYSERASPVALTYRTYRALAGLYLEASNTYRLYSASNGLYGLYPHEALAGLYASNTYRALAPHELYSTYRALALYSHISALAASNVALGLYARGASPALAPHEGLLEPHELELEGLYSERGLYSERASPGLNALALYSGLYTHRASPPRLELYSASNHISGLNVALHISARGLETHRGLYGLYTYRGLGLGLYGLYLEASNLEALALEALAALAGLNLEASPLESERGLASNALAASPLYSTHRLYSASNSERTHRTHRGLILEALAALATHRALASERTYRARGPHEGLYASNALAVALPRARGILESERTYRALAHISGLYPHEASPLEILEGLARGGLYLYSLYSGLYGLASNTHRSERTYRASPGLNILEILEALAGLYVALASPTYRASPPHESERLYSARGTHRSERALAILEVALSERGLYALATRPLELYSARGASNTHRGLYILEGLYASNTYRTHRGLNILEASNALAALASERVALGLYLEARGHISLYSPHE

SEQ ID NO: 15 - DNA, 1119 bp, Neisseria meningitidis (group B)
atggatgtcagcctatacggcgaaatcaaagccggcgtggaaggcaggaacatccaggcgcaattgaccgagcagccccaagtaactaacggtgtgcaaggcaatcaggtaaaagttactaaggccaaaagccgcatcaggacgaaaatcagcgatttcggctcgtttatcggctttaaggggagcgaggatttgggcgaagggctgaaggctgtttggcagcttgagcaagacgtatccgttgccggcggcggcgcgtcccagtggggcaacagggaatcctttatcggcttggcaggcgaattcggtacgctgcgcgccggtcgcgttgcaaatcagtttgacgatgccagccaagccattaatccttgggacagcaataatgatgtggcttcgcaattgggtattttcaaacgccacgacgatatgccggtttccgtacgctacgattctccggaattttccggtttcagcggcagcgtccaattcgttccggctcaaaacagcaagtccgcctataagccggcttattatactaaggatacaaacaataatcttactctcgttccggctgttgtcggcaagcccggatcggatgtgtattatgccggtctgaattacaaaaatggcggttttgccgggaactatgcctttaaatatgcgagacacgccaatgtcggacgtaatgcttttgagttgttcttgatcggcagcgcgacgagtgatgaagccaaaggtaccgatcccttgaaaaaccatcaggtacaccgcctgacgggcggctatgaggaaggcggcttgaatctcgccttggcggctcagttggatttgtctgaaaatgccgacaaaaccaaaaacagtacgaccgaaattgccgccactgcttcctaccgcttcggtaatgcagttccgcgcatcagctatgcccatggtttcgactttatcgaacgcggtaaaaaaggcgaaaataccagctacgatcaaatcatcgccggcgttgattatgatttttccaaacgcacttccgccatcgtgtctggcgcttggctgaaacgcaataccggcatcggcaactacactcaaattaatgccgcctccgtcggtttgcgccacaaattc

SEQ ID NO: 16 - protein, 1070 aa, Neisseria meningitidis (group B)
METASPVALSERLETYRGLYGLILELYSALAGLYVALGLGLYARGASNILEGLNALAGLNLETHRGLGLNPRGLNVALTHRASNGLYVALGLNGLYASNGLNVALLYSVALTHRLYSALALYSSERARGILEARGTHRLYSILESERASPPHEGLYSERPHEILEGLYPHELYSGLYSERGLASPLEGLYGLGLYLELYSALAVALTRPGLNLEGLGLNASPVALSERVALALAGLYGLYGLYALASERGLNTRPGLYASNARGGLSERPHEILEGLYLEALAGLYGLPHEGLYTHRLEARGALAGLYARGVALALAASNGLNPHEASPASPALASERGLNALAILEASNPRTRPASPSERASNASNASPVALALASERGLNLEGLYILEPHELYSARGHISASPASPMETPRVALSERVALARGTYRASPSERPRGLPHESERGLYPHESERGLYSERVALGLNPHEVALPRALAGLNASNSERLYSSERALATYRLYSPRALATYRTYRTHRLYSASPTHRASNASNASNLETHRLEVALPRALAVALVALGLYLYSPRGLYSERASPVALTYRTYRALAGLYLEASNTYRLYSASNGLYGLYPHEALAGLYASNTYRALAPHELYSTYRALAARGHISALAASNVALGLYARGASNALAPHEGLLEPHELEILEGLYSERALATHRSERASPGLALALYSGLYTHRASPPRLELYSASNHISGLNVALHISARGLETHRGLYGLYTYRGLGLGLYGLYLEASNLEALALEALAALAGLNLEASPLESERGLASNALAASPLYSTHRLYSASNSERTHRTHRGLILEALAALATHRALASERTYRARGPHEGLYASNALAVALPRARGILESERTYRALAHISGLYPHEASPPHEILEGLARGGLYLYSLYSGLYGLASNTHRSERTYRASPGLNILEILEALAGLYVALASPTYRASPPHESERLYSARGTHRSERALAILEVALSERGLYALATRPLELYSARGASNTHRGLYILEGLYASNTYRTHRGLNILEASNALAALASERVALGLYLEARGHISLYSPHE

SEQ ID NO: 17 - DNA, 1113 bp, Neisseria meningitidis (group B)
atggatgtcagcctatacggcgaaatcaaagccggcgtggaaggcaggaactaccagctgcaattgactgaagcacaagccgctaacggtggagcgagcggtcaggtaaaagttactaaggccaaaagccgcatcaggacgaaaatcagtgatttcggctcgtttatcggctttaaggggagtgaggatttgggcgacgggctgaaggctgtttggcagcttgagcaagacgtatccgttgccggcggcggcgcgacccagtggggcaacagggaatcctttatcggcttggcaggcgaattcggtacgctgcgcgccggtcgcgttgcgaatcagtttgacgatgccagccaagccattgatccttgggacagcaataatgatgtggcttcgcaattgggtattttcaaacgccacgacgacatgccggtttccgtacgctacgattcccccgaattttccggtttcagcggcagcgttcaattcgttccgatccaaaacagcaagtccgcctatacgccggcttattatactaaggatacaaacaataatcttactctcgttccggctgttgtcggcaagcccggatcggatgtgtattatgccggtctgaattacaaaaatggcggttttgccgggaactatgcctttaaatatgcgagacacgccaatgtcggacgtaatgcttttgagttgttcttgatcggcagcgggagtgatcaagccaaaggtaccgatcccttgaaaaaccatcaggtacaccgtctgacgggcggctatgaggaaggcggcttgaatctcgccttggcggctcagttggatttgtctgaaaatggcgacaaaaccaaaaacagtacgaccgaaattgccgccactgcttcctaccgcttcggtaatgcagttccacgcatcagctatgcccatggtttcgactttatcgaacgcggtaaaaaaggcgaaaataccagctacgatcaaatcatcgccggcgttgattatgatttttccaaacgcacttccgccatcgtgtctggcgcttggctgaagcgcaataccggcatcggcaactacactcaaattaatgccgcctccgtcggtttgcgccacaaattc

SEQ ID NO: 18 - protein, 371 aa, Neisseria meningitidis (group B)
MDVSLYGEIKAGVEGRNYQLQLTEAQAANGGASGQVKVTKAKSRIRTKISDFGSFIGFKGSEDLGDGLKAVWQLEQDVSVAGGGATQWGNRESFIGLAGEFGTLRAGRVANQFDDASQAIDPWDSNNDVASQLGIFKRHDDMPVSVRYDSPEFSGFSGSVQFVPIQNSKSAYTPAYYTKDTNNNLTLVPAVVGKPGSDVYYAGLNYKNGGFAGNYAFKYARHANVGRNAFELFLIGSGSDQAKGTDPLKNHQVHRLTGGYEEGGLNLALAAQLDLSENGDKTKNSTTEIAATASYRFGNAVPRISYAHGFDFIERGKKGENTSYDQIIAGVDYDFSKRTSAIVSGAWLKRNTGIGNYTQINAASVGLRHKF

SEQ ID NO: 19 - DNA, 1110 bp, Neisseria meningitidis (group B)
atggatgtcagcctgtacggcgaaatcaaagccggcgtggaaggcaacaacattcagctgcaattgaccgaaccaccctcaaaaggtcagacgggcaataaagttactaagggcaaaagccgcatcaggacgaaaatcaacgatttcggctcgtttatcggctttaaggggagcgaggatttgggcgaagggctgaaggctgtttggcagcttgagcaagacgtatccgttgccggcggcggcgcgacccagtggggcaacagggaatcctttatcggcttggcaggcgaattcggcacgctgcgcgccggtcgcgttgcaaatcagtttgacgatgccagccaagccattgatccttgggacagcaacaatgatgtggcttcgcaattgggtattttcaaacgccacgacgatatgccggtttctgtacgctacgactctccggacttttccggtttcagcggcagcgtccaattcgttccggcccaaaacagcaaatccgcctatacgccggctacttatactgtggatagtagtggtgttgttactcccgttcctgctgttgtcggcaagcccggatcggatgtgtattatgccggtctgaattacaaaaatggcggttttgccgggaactatgcctttaaatacgcgaaacacgccaatgtgggccgtgatgcttttaatttgttcttgcttgggcgcatcggcgaggatgatgaagccaaaggtaccgatcccttgaaaaaccatcaggtacaccgcctgacgggcggctatgaggaaggcggcttgaatctcgccttggcggctcagttggatttgtctgaaaatggcgacaaaaccaaaaacagtacgaccgaaattgccgccactgcttcctaccgcttcgggaatgcagttccacgcatcagctatgcccatggtttcgactttatcgaacgcggtaaaaaaggcgaacataccagctacgatcaaatcatcgccggcgttgattatgatttttccaaacgcacttccgccatcgtgtctggtgcttggctgaaacgcaataccggcatcggcaactacactcaaattaatgccgcctccgtcggtttgcgccacaaattc

SEQ ID NO: 20 - protein, 1060 aa, Neisseria meningitidis (group B)
METASPVALSERLETYRGLYGLILELYSALAGLYVALGLGLYASNASNILEGLNLEGLNLETHRGLPRPRSERLYSGLYGLNTHRGLYASNLYSVALTHRLYSGLYLYSSERARGILEARGTHRLYSILEASNASPPHEGLYSERPHEILEGLYPHELYSGLYSERGLASPLEGLYGLGLYLELYSALAVALTRPGLNLEGLGLNASPVALSERVALALAGLYGLYGLYALATHRGLNTRPGLYASNARGGLSERPHEILEGLYLEALAGLYGLPHEGLYTHRLEARGALAGLYARGVALALAASNGLNPHEASPASPALASERGLNALAILEASPPRTRPASPSERASNASNASPVALALASERGLNLEGLYILEPHELYSARGHISASPASPMETPRVALSERVALARGTYRASPSERPRASPPHESERGLYPHESERGLYSERVALGLNPHEVALPRALAGLNASNSERLYSSERALATYRTHRPRALATHRTYRTHRVALASPSERSERGLYVALVALTHRPRVALPRALAVALVALGLYLYSPRGLYSERASPVALTYRTYRALAGLYLEASNTYRLYSASNGLYGLYPHEALAGLYASNTYRALAPHELYSTYRALALYSHISALAASNVALGLYARGASPALAPHEASNLEPHELELEGLYARGILEGLYGLASPASPGLALALYSGLYTHRASPPRLELYSASNHISGLNVALHISARGLETHRGLYGLYTYRGLGLGLYGLYLEASNLEALALEALAALAGLNLEASPLESERGLASNGLYASPLYSTHRLYSASNSERTHRTHRGLILEALAALATHRALASERTYRARGPHEGLYASNALAVALPRARGILESERTYRALAHISGLYPHEASPPHEILEGLARGGLYLYSLYSGLYGLHISTHRSERTYRASPGLNILEILEALAGLYVALASPTYRASPPHESERLYSARGTHRSERALAILEVALSERGLYALATRPLELYSARGASNTHRGLYILEGLYASNTYRTHRGLNILEASNALAALASERVALGLYLEARGHISLYSPHE

SEQ ID NO: 21 - DNA, 45 bp, Neisseria meningitidis (group B)
cgcgagatctcatatggatgtcagcctatacggcgaaatcaaagc

SEQ ID NO: 22 - DNA, 46 bp, Neisseria meningitidis (group B)
cgtcggtttgcgccacaaattctaatgagtgactgaagatctcgcg

SEQ ID NO: 23 - DNA, 70 bp, Neisseria meningitidis (group B)
cgcgagatctcatatggatgtcagcctatacggcgaaatcaaagccggcgtggaaggcaggaactaccag

SEQ ID NO: 24 - DNA, 1110 bp, Neisseria meningitidis (group B)
atggatgtcagcctgtacggcgaaatcaaagccggcgtggaaggcaggaacatccagctgcaattgaccgagcagccctcaaaggctcaaggtcaaacgaacaatcaggtaaaagttactaaggcaaaaagccgcatcaggacgaaaatcagcgatttcggctcgtttatcggctttaaggggagtgaggatttgggcgacgagctgaaggctgtttggcagcttgagcaagacgtatccgttgccggcggcggcgcgacccgttggggcaatagggaatcctttgtcggcttggcaggcgaattcggcacgctgcgcgccggccgcgttgcgaatcagtttgacgatgccagccaagccattgatccttgggacagcaacaatgatgtggcttcgcaattgggtattttcaaacgccacgacgatatgccggtttccgtacgctacgactctccggacttttccggtttcagcggcagcgtccaattcgttccgatccaaaacagcaagtccgcctataagccggcttatgtggatgagaagaagatggttcatgcggctgttgtcggcaagcccggatcggatgtgtattatgccggtctgaattacaaaaatggcggatttgccgggagctatgcctttaaatatgcgagacacgccaatgtcggacgtgatgcttttgagttgttcttgctcggcagcacgagtgatgaagccaaaggtaccgatcccttgaaaaaccatcaggtacaccgcctgacgggcggctatgaggaaggcggcttgaatctcgccttggcggcccagttggatttgtctgaaaatggcgacaaagccaaaaccaaaaacagtacgaccgaaattgccgcgactgcttcctaccgcttcggtaatgcagttccacgcatcagctatgcccatggtttcgacttgatcgaacgcggtaaaaaaggcgaaaataccagctacgatcaaatcatcgccggcgttgattatgatttttccaaacgcacttccgccatcgtgtctggcgcttggctgaaacgcaataccggcatcggcaactacactcaaattaatgccgcctccgtcggtttgcgccacaaattc

SEQ ID NO: 25 - protein, 370 aa, Neisseria meningitidis (group B)
MAVSLTGGILAGVGGAAIGLGLTGGPSLAGGGTAAGVLVTLALSAIATLISAPGSPIGPLGSGALGAGLLAVTGLGGAVSVAGGGATATGAAGSPVGLAGGPGTLAAGAVAAGPAAASGAIAPTASAAAVASGLGIPLAHAAMPVSVATASPAPSGPSGSVGPVPIGASLSATLPATVAGLLMVHAAVVGLPGSAVTTAGLATLAGGPAGSTAPLTAAHAAVGAAAPGLPLLGSTSAGALGTAPLLAHGVHALTGGTGGGGLALALAAGLALSGAGALALTLASTTGIAATASTAPGAAVPAISTAHGPALIGAGLLGGATSTAGIIAGVATAPSLATSAIVSGATLLAATGIGATTGIAAASVGLAHLP

SEQ ID NO: 26 - protein, 10 aa, Neisseria meningitidis (group B)
  misc_feature (5)..(5): Xaa can be any naturally occurring amino acid
ADIGXGLADA

SEQ ID NO: 27 - protein, 10 aa, Neisseria meningitidis (group B)
  misc_feature (3)..(3): Xaa can be any naturally occurring amino acid
IGXGLADALT

SEQ ID NO: 28 - protein, 10 aa, Neisseria meningitidis (group B)
SLNTGKLKND

SEQ ID NO: 29 - protein, 17 aa, Neisseria meningitidis (group B)
  misc_feature (12)..(12): Xaa can be any naturally occurring amino acid
SLNTGKLKNDKXSRFDF

SEQ ID NO: 30 - protein, 9 aa, Neisseria meningitidis (group B)
  misc_feature (6)..(6): Xaa can be any naturally occurring amino acid
SGEFQXYKQ

SEQ ID NO: 31 - protein, 8 aa, Neisseria meningitidis (group B)
  misc_feature (6)..(6): Xaa can be any naturally occurring amino acid
IEHLKXPE

SEQ ID NO: 32 - protein, 10 aa, Neisseria meningitidis (group B)
  misc_feature (10)..(10): Xaa can be any naturally occurring amino acid
GGGVAADIGX

SEQ ID NO: 33 - protein, 9 aa, Neisseria meningitidis (group B)
SGEFQIYKQ

SEQ ID NO: 34 - protein, 10 aa, Neisseria meningitidis (group B)
HSAVVALQIE

SEQ ID NO: 35 - protein, 10 aa, Neisseria meningitidis (group B)
EKINNPDKID

SEQ ID NO: 36 - protein, 10 aa, Neisseria meningitidis (group B)
SLINQRSFLV

SEQ ID NO: 37 - protein, 10 aa, Neisseria meningitidis (group B)
SGLGGEHTAF

SEQ ID NO: 38 - protein, 10 aa, Neisseria meningitidis (group B)
GEHTAFNQLP

SEQ ID NO: 39 - protein, 11 aa, Neisseria meningitidis (group B)
SFLVSGLGGEH

SEQ ID NO: 40 - protein, 34 aa, Neisseria meningitidis (group B)
EKINNPDKIDSLINQRSFLVSGLGGEHTAFNQLP

SEQ ID NO: 41 - protein, 10 aa, Neisseria meningitidis (group B)
GKAEYHGKAF

SEQ ID NO: 42 - protein, 10 aa, Neisseria meningitidis (group B)
YHGKAFSSDD

SEQ ID NO: 43 - protein, 14 aa, Neisseria meningitidis (group B)
GKAEYHGKAFSSDD

SEQ ID NO: 44 - protein, 10 aa, Neisseria meningitidis (group B)
IEHLKTPEQN

SEQ ID NO: 45 - protein, 10 aa, Neisseria meningitidis (group B)
KTPEQNVELA

SEQ ID NO: 46 - protein, 14 aa, Neisseria meningitidis (group B)
IEHLKTPEQNVELA

SEQ ID NO: 47 - protein, 10 aa, Neisseria meningitidis (group B)
AELKADEKSH

SEQ ID NO: 48 - protein, 10 aa, Neisseria meningitidis (group B)
AVILGDTRYG

SEQ ID NO: 49 - protein, 20 aa, Neisseria meningitidis (group B)
AELKADEKSHAVILGDTRYG

SEQ ID NO: 50 - protein, 10 aa, Neisseria meningitidis (group B)
EEKGTYHLAL

SEQ ID NO: 51 - protein, 14 aa, Neisseria meningitidis (group B)
KINNPDKIDSLINQ

SEQ ID NO: 52 - protein, 10 aa, Neisseria meningitidis (group B)
DEKSHAVILG

SEQ ID NO: 53 - protein, 10 aa, Neisseria meningitidis (group B)
KIGEKVHEIG

SEQ ID NO: 54 - protein, 10 aa, Neisseria meningitidis (group B)
LITLESGEFQ

SEQ ID NO: 55 - protein, 10 aa, Neisseria meningitidis (group B)
SALTALQTEQ

SEQ ID NO: 56 - protein, 10 aa, Neisseria meningitidis (group B)
FQVYKQSHSA

SEQ ID NO: 57 - protein, 26 aa, Neisseria meningitidis (group B)
LITLESGEFQVYKQSHSALTALQTEQ

SEQ ID NO: 58 - protein, 10 aa, Neisseria meningitidis (group B)
IGDIAGEHTS

SEQ ID NO: 59 - protein, 10 aa, Neisseria meningitidis (group B)
EHTSFDKLPK

SEQ ID NO: 60 - protein, 16 aa, Neisseria meningitidis (group B)
IGDIAGEHTSFDKLPK

SEQ ID NO: 61 - protein, 10 aa, Neisseria meningitidis (group B)
ATYRGTAFGS

SEQ ID NO: 62 - protein, 10 aa, Neisseria meningitidis (group B)
DDAGGKLTYT

SEQ ID NO: 63 - protein, 10 aa, Neisseria meningitidis (group B)
IDFAAKQGHG

SEQ ID NO: 64 - protein, 10 aa, Neisseria meningitidis (group B)
KIEHLKSPEL

SEQ ID NO: 65 - protein, 42 aa, Neisseria meningitidis (group B)
ATYRGTAFGSDDAGGKLTYTIDFAAKQGHGKIEHLKSPELNV

SEQ ID NO: 66 - protein, 10 aa, Neisseria meningitidis (group B)
HAVISGSVLY

SEQ ID NO: 67 - protein, 10 aa, Neisseria meningitidis (group B)
KGSYSLGIFG

SEQ ID NO: 68 - protein, 10 aa, Neisseria meningitidis (group B)
VLYNQDEKGS

SEQ ID NO: 69 - protein, 24 aa, Neisseria meningitidis (group B)
HAVISGSVLYNQDEKGSYSLGIFG

SEQ ID NO: 70 - protein, 10 aa, Neisseria meningitidis (group B)
AQEVAGSAEV

SEQ ID NO: 71 - protein, 10 aa, Neisseria meningitidis (group B)
IHHIGLAAKQ

SEQ ID NO: 72 - protein, 10 aa, Neisseria meningitidis (group B)
VETANGIHHI

SEQ ID NO: 73 - protein, 25 aa, Neisseria meningitidis (group B)
AQEVAGSAEVETANGIHHIGLAAKQ

SEQ ID NO: 74 - protein, 22 aa, Neisseria meningitidis (group B)
VAGSAEVETANGIHHIGLAAKQ

SEQ ID NO: 75 - protein, 10 aa, Neisseria meningitidis (group B)
MVAKRQFRIG

SEQ ID NO: 76 - protein, 13 aa, Neisseria meningitidis (group B)
DIAGEHTSFDKLP

SEQ ID NO: 77 - protein, 10 aa, Neisseria meningitidis (group B)
YTIDFAAKQG

SEQ ID NO: 78 - protein, 13 aa, Neisseria meningitidis (group B)
GKIEHLKSPELNV

SEQ ID NO: 79 - protein, 12 aa, Neisseria meningitidis (group B)
HAVISGSVLYNQ

SEQ ID NO: 80 - protein, 10 aa, Neisseria meningitidis (group B)
AQEVAGSAEV

SEQ ID NO: 81 - protein, 254 aa, Artificial
  Consensus protein sequence of Neisseria meningitidis ORF2086
  misc_feature (9)..(9): Xaa can be any naturally occurring amino acid
  misc_feature (14)..(14): Xaa can be any naturally occurring amino acid
  misc_feature (22)..(22): Xaa can be any naturally occurring amino acid
  misc_feature (24)..(24): Xaa can be any naturally occurring amino acid
  misc_feature (26)..(26): Xaa can be any naturally occurring amino acid
  misc_feature (30)..(30): Xaa can be any naturally occurring amino acid
  misc_feature (32)..(32): Xaa can be any naturally occurring amino acid
  misc_feature (37)..(38): Xaa can be any naturally occurring amino acid
  misc_feature (40)..(42): Xaa can be any naturally occurring amino acid
  misc_feature (44)..(45): Xaa can be any naturally occurring amino acid
  misc_feature (47)..(47): Xaa can be any naturally occurring amino acid
  misc_feature (49)..(49): Xaa can be any naturally occurring amino acid
  misc_feature (57)..(59): Xaa can be any naturally occurring amino acid
  misc_feature (73)..(73): Xaa can be any naturally occurring amino acid
  misc_feature (79)..(81): Xaa can be any naturally occurring amino acid
  misc_feature (83)..(83): Xaa can be any naturally occurring amino acid
  misc_feature (87)..(88): Xaa can be any naturally occurring amino acid
  misc_feature (92)..(92): Xaa can be any naturally occurring amino acid
  misc_feature (98)..(98): Xaa can be any naturally occurring amino acid
  misc_feature (102)..(102): Xaa can be any naturally occurring amino acid
  misc_feature (106)..(107): Xaa can be any naturally occurring amino acid
  misc_feature (111)..(111): Xaa can be any naturally occurring amino acid
  misc_feature (113)..(126): Xaa can be any naturally occurring amino acid
  misc_feature (128)..(128): Xaa can be any naturally occurring amino acid
  misc_feature (130)..(135): Xaa can be any naturally occurring amino acid
  misc_feature (140)..(140): Xaa can be any naturally occurring amino acid
  misc_feature (142)..(143): Xaa can be any naturally occurring amino acid
  misc_feature (146)..(148): Xaa can be any naturally occurring amino acid
  misc_feature (150)..(150): Xaa can be any naturally occurring amino acid
  misc_feature (152)..(152): Xaa can be any naturally occurring amino acid
  misc_feature (154)..(154): Xaa can be any naturally occurring amino acid
  misc_feature (157)..(157): Xaa can be any naturally occurring amino acid
  misc_feature (161)..(162): Xaa can be any naturally occurring amino acid
  misc_feature (164)..(164): Xaa can be any naturally occurring amino acid
  misc_feature (166)..(166): Xaa can be any naturally occurring amino acid
  misc_feature (168)..(168): Xaa can be any naturally occurring amino acid
  misc_feature (172)..(173): Xaa can be any naturally occurring amino acid
  misc_feature (177)..(177): Xaa can be any naturally occurring amino acid
  misc_feature (179)..(179): Xaa can be any naturally occurring amino acid
  misc_feature (185)..(185): Xaa can be any naturally occurring amino acid
  misc_feature (188)..(188): Xaa can be any naturally occurring amino acid
  misc_feature (191)..(191): Xaa can be any naturally occurring amino acid
  misc_feature (194)..(197): Xaa can be any naturally occurring amino acid
  misc_feature (199)..(199): Xaa can be any naturally occurring amino acid
  misc_feature (203)..(203): Xaa can be any naturally occurring amino acid
  misc_feature (208)..(208): Xaa can be any naturally occurring amino acid
  misc_feature (210)..(212): Xaa can be any naturally occurring amino acid
  misc_feature (214)..(216): Xaa can be any naturally occurring amino acid
  misc_feature (220)..(220): Xaa can be any naturally occurring amino acid
  misc_feature (222)..(222): Xaa can be any naturally occurring amino acid
  misc_feature (224)..(226): Xaa can be any naturally occurring amino acid
  misc_feature (228)..(229): Xaa can be any naturally occurring amino acid
  misc_feature (233)..(233): Xaa can be any naturally occurring amino acid
  misc_feature (236)..(236): Xaa can be any naturally occurring amino acid
  misc_feature (238)..(238): Xaa can be any naturally occurring amino acid
  misc_feature (240)..(245): Xaa can be any naturally occurring amino acid
  misc_feature (247)..(247): Xaa can be any naturally occurring amino acid
  misc_feature (249)..(250): Xaa can be any naturally occurring amino acid
  misc_feature (252)..(252): Xaa can be any naturally occurring amino acid
CSSGGGGVXADIGXGLADALTXPXDXKDKXLXSLTLXXSXXXNXXLXLXAQGAEKTXXXGDSLNTGKLKNDKXSRFDFXXXIXVDGXXITLXSGEFQXYKQXHSAXXALQXEXXXXXXXXXXXXXXRXFXXXXXXGEHTXFXXLPXXXAXYXGXAFXSDDXXGXLXYXIDFXXKQGXGXIEHLKXPEXNVXLAXXXXKXDEKXHAVIXGXXXYXXXEKGXYXLXXXGXXAQEXAGXAXVXXXXXXHXIXXAXKQ

SEQ ID NO: 82 - protein, 254 aa, Artificial
  ORF2086 protein, subfamily A
  misc_feature (14)..(14): Xaa can be any naturally occurring amino acid
  misc_feature (22)..(22): Xaa can be any naturally occurring amino acid
  misc_feature (24)..(24): Xaa can be any naturally occurring amino acid
  misc_feature (26)..(26): Xaa can be any naturally occurring amino acid
  misc_feature (30)..(30): Xaa can be any naturally occurring amino acid
  misc_feature (32)..(32): Xaa can be any naturally occurring amino acid
  misc_feature (37)..(38): Xaa can be any naturally occurring amino acid
  misc_feature (40)..(42): Xaa can be any naturally occurring amino acid
  misc_feature (44)..(45): Xaa can be any naturally occurring amino acid
  misc_feature (47)..(47): Xaa can be any naturally occurring amino acid
  misc_feature (49)..(49): Xaa can be any naturally occurring amino acid
  misc_feature (57)..(59): Xaa can be any naturally occurring amino acid
  misc_feature (73)..(73): Xaa can be any naturally occurring amino acid
  misc_feature (79)..(81): Xaa can be any naturally occurring amino acid
  misc_feature (83)..(83): Xaa can be any naturally occurring amino acid
  misc_feature (88)..(88): Xaa can be any naturally occurring amino acid
  misc_feature (92)..(92): Xaa can be any naturally occurring amino acid
  misc_feature (102)..(102): Xaa can be any naturally occurring amino acid
  misc_feature (146)..(146): Xaa can be any naturally occurring amino acid
  misc_feature (161)..(162): Xaa can be any naturally occurring amino acid
  misc_feature (164)..(164): Xaa can be any naturally occurring amino acid
  misc_feature (166)..(166): Xaa can be any naturally occurring amino acid
  misc_feature (168)..(168): Xaa can be any naturally occurring amino acid
  misc_feature (172)..(173): Xaa can be any naturally occurring amino acid
  misc_feature (177)..(177): Xaa can be any naturally occurring amino acid
  misc_feature (179)..(179): Xaa can be any naturally occurring amino acid
  misc_feature (194)..(194): Xaa can be any naturally occurring amino acid
  misc_feature (215)..(215): Xaa can be any naturally occurring amino acid
  misc_feature (226)..(226): Xaa can be any naturally occurring amino acid
  misc_feature (236)..(236): Xaa can be any naturally occurring amino acid
  misc_feature (242)..(242): Xaa can be any naturally occurring amino acid
  misc_feature (249)..(249): Xaa can be any naturally occurring amino acid
  misc_feature (252)..(252): Xaa can be any naturally occurring amino acid
CSSGGGGVAADIGXGLADALTXPXDXKDKXLXSLTLXXSXXXNXXLXLXAQGAEKTXXXGDSLNTGKLKNDKXSRFDFXXXIXVDGQXITLXSGEFQIYKQXHSAVVALQIEKINNPDKIDSLINQRSFLVSGLGGEHTAFNQLPXGKAEYHGKAFSSDDXXGXLXYXIDFXXKQGXGXIEHLKTPEQNVELAXAELKADEKSHAVILGDTRYGXEEKGTYHLALXGDRAQEIAGXATVKIXEKVHEIXIAXKQ

SEQ ID NO: 83 - protein, 255 aa, Artificial
  ORF2086 protein, subfamily B
  misc_feature (9)..(9): Xaa can be any naturally occurring amino acid
  misc_feature (14)..(14): Xaa can be any naturally occurring amino acid
  misc_feature (30)..(30): Xaa can be any naturally occurring amino acid
  misc_feature (32)..(32): Xaa can be any naturally occurring amino acid
  misc_feature (37)..(38): Xaa can be any naturally occurring amino acid
  misc_feature (40)..(42): Xaa can be any naturally occurring amino acid
  misc_feature (44)..(45): Xaa can be any naturally occurring amino acid
  misc_feature (47)..(47): Xaa can be any naturally occurring amino acid
  misc_feature (49)..(49): Xaa can be any naturally occurring amino acid
  misc_feature (87)..(87): Xaa can be any naturally occurring amino acid
  misc_feature (114)..(114): Xaa can be any naturally occurring amino acid
  misc_feature (117)..(117): Xaa can be any naturally occurring amino acid
  misc_feature (119)..(119): Xaa can be any naturally occurring amino acid
  misc_feature (121)..(121): Xaa can be any naturally occurring amino acid
  misc_feature (128)..(128): Xaa can be any naturally occurring amino acid
  misc_feature (130)..(130): Xaa can be any naturally occurring amino acid
  misc_feature (147)..(149): Xaa can be any naturally occurring amino acid
  misc_feature (192)..(192): Xaa can be any naturally occurring amino acid
  misc_feature (195)..(196): Xaa can be any naturally occurring amino acid
  misc_feature (204)..(204): Xaa can be any naturally occurring amino acid
  misc_feature (229)..(230): Xaa can be any naturally occurring amino acid
CSSGGGGVXADIGXGLADALTAPLDHKDKXLXSLTLXXSXXXNXXLXLXAQGAEKTYGNGDSLNTGKLKNDKVSRFDFIRQIEVDGXLITLESGEFQVYKQSHSALTALQTEQXQDXEXSXKMVAKRXFXIGDIAGEHTSFDKLPKXXXATYRGTAFGSDDAGGKLTYTIDFAAKQGHGKIEHLKSPELNVXLAXXYIKPDEKXHAVISGSVLYNQDEKGSYSLGIFGXXAQEVAGSAEVETANGIHHIGLAAKQ

(19) United States
(12) Patent Application Publication
(10) Pub. No.: US 2007/0148729 A1
(43) Pub. Date: Jun. 28, 2007

(54) Methods for increasing neisseria protein expression and compositions thereof

(76) Inventors: John Erwin Farley, Chapel Hill, NC (US); Susan Kay Holseth, Suffern, NY (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 10/542,284

(22) PCT Filed: Jan. 13, 2004
(86) PCT No.: PCT/US04/00800

§ 371 Date: Nov. 15, 2006

Related U.S. Application Data
(60) Provisional application No. 60/440,244, filed on Jan. 15, 2003.

(51) Int. Cl. C12P 21/06; C07H 21/04; C12N 15/74; C12N 1/21

(52) U.S. Cl. 435/69.1; 435/252.3; 435/471; 530/350; 536/23.7

(57) ABSTRACT

The present invention broadly relates to polynucleotide sequences encoding porin polypeptides of Neisseria. More particularly, the invention relates to newly identified nucleic acid sequence mutations in polynucleotides encoding PorA polypeptides of Neisseria meningitidis, wherein these sequence mutations result in increased expression levels of PorA polypeptides.

### FIELD OF THE INVENTION

The invention relates to polynucleotide sequences encoding porin polypeptides of Neisseria. More particularly, the invention relates to newly identified nucleic acid sequence mutations in polynucleotides encoding PorA polypeptides of Neisseria meningitidis, wherein the sequence mutations result in increased expression levels of PorA polypeptides.

### BACKGROUND OF THE INVENTION

Neisseria meningitidis is a major cause of death and morbidity throughout the world. Neisseria meningitidis causes both endemic and epidemic diseases, principally meningitis and meningococcemia (Schwartz et al., 1989), with incidences as high as 1,000 per 100,000 having been reported during epidemics in sub-Saharan Africa (Riedo et al., 1995). In fact, Neisseria meningitidis is one of the most common causes of bacterial meningitis in the United States, accounting for approximately 20-25% of all cases (Dawson et al., 1999). Without antibiotic treatment, the mortality of Neisseria meningitidis infection can be as high as 85% and even with this treatment, it still remains at approximately 10%. In addition, patients treated by antibiotics can still suffer serious and permanent neurologic deficiencies.

Isolates of Neisseria meningitidis are subdivided into serological groups according to the presence of capsular antigens. Currently, 12 serogroups are recognized, with serogroups A, B, C, Y, and W-135 being most commonly found. Within serogroups, serotypes, serosubtypes and immunotypes can be identified by outer membrane proteins and lipopolysaccharide (Frasch et al., 1985(a)). It has been well documented that serum bactericidal activity is the major defense mechanism against Neisseria meningitidis and that protection against invasion by the bacteria correlates with the presence in the serum of anti-meningococcal antibodies (Goldschneider et al., 1969).

The capsular polysaccharide immunogenic compositions presently available are not effective against all Neisseria meningitidis isolates and do not effectively induce the production of protective antibodies in young infants, who are the principal victims of this disease (Frasch, 1989; Reingold et al., 1985; Zollinger, 1990). The capsular polysaccharides of serogroups A, C, Y and W-1 35 are presently used in immunogenic compositions against Neisseria meningitidis. These polysaccharide compositions are effective in the short term, however the vaccinated subjects do not develop an immunological memory, so they must be revaccinated within a three-year period to maintain their level of resistance. The introduction of the meningococcal C conjugate vaccine has overcome this limitation and provides long term protection.

In contrast to pneumococcal immunogenic compositions, meningococcal polysaccharide immunogenic compositions have been greatly simplified by the fact that fewer polysaccharides are required. In fact, groups A, B, C, Y and W135 are responsible for a majority of meningococcal meningitis. Some success in the prevention of group A and C meningococcal meningitis was achieved using a bivalent polysaccharide immunogenic composition (Gotschlich et al., 1969; Artenstein et al., 1970). However, there has been a need to augment this composition because infants fail to respond to the polysaccharide vaccine, and because a significant proportion of cases of meningococcal meningitis are due to groups other than A and C. Although Y and W135 are now included in the polysaccharide vaccine, B is not.

Group B is of particular epidemiologic importance. The inclusion of the group B polysaccharide in the immunogenic composition remains a special problem. The group B meningococcal polysaccharide is poorly immunogenic in man (Wyle et al., 1972). The group B capsular polysaccharides (CPs) consist of polymers of N-acetylneuraminic acid known as polysialic acid (PSA). PSA is carried on human neural cell adhesion molecules (NCAM) of fetal and newborn tissues, and on selected adult tissues (Seki and Arai, 1993). Thus, the structure is recognized as “self” by the human immune system and in consequence, the production of antibody specific for this structure is suppressed. Because of this molecular mimicry, an immunogenic composition based on the native group B CPs could raise antibody directed against the poly N-acetylneuraminic acid moiety, and might induce autoimmune disease.

Presently, no effective immunogenic composition against serogroup B isolates is available even though these organisms are one of the primary causes of meningococcal diseases in developed countries. Indeed, the serogroup B polysaccharide is not a good immunogen, inducing only a poor response of IgM of low specificity which is not protective (Gotschlich et al., 1969; Skevakis et al., 1984; Zollinger, 1979). Furthermore, the presence of closely similar, crossreactive structures in the glycoproteins of neonatal human brain tissue (Finne et al., 1983) might discourage attempts at improving the immunogenicity of serogroup B polysaccharide. To obtain a more effective immunogenic composition, other Neisseria meningitidis surface antigens such as lipopolysaccharide, pili proteins and proteins present in the outer membrane are under investigation.

The outer membranes of Neisseria species are semi-permeable, which allow free flow access and escape of small molecular weight substances to and from the periplasmic space, but retard molecules of larger size (Heasley et al., 1980; Douglas et al., 1981). One of the mechanisms whereby this is accomplished is the inclusion within these membranes of proteins which have been collectively named porins. These proteins are made up of three identical polypeptide chains (i.e., homotrimers) (Jones et al., 1980; McDade Jr. and Johnston, 1980) and in their native trimer conformation form water filled, voltage-dependent channels within the outer membrane of the bacteria or other membranes to which they have been introduced (Lynch et al., 1984(a); Lynch et al., 1984(b); Young et al., 1983; Mauro et al., 1988; Young et al., 1986). Because of the relative abundance of these proteins within the outer membrane, these protein antigens have been used to subgroup Neisseria meningitidis into several serotypes and serosubtypes for epidemiological purposes (Frasch et al., 1985(b); Knapp et al., 1985). These Neisseria porins have been the subject of considerable investigation (James and Heckels, 1981; Judd, 1988; Blake and Gotschlich, 1982; Wetzler, et al., 1988), and many have been cloned and sequenced (Gotschlich et al., 1987; McGuinness et al., 1990; Carbonetti and Sparling, 1987; Feavers et al., 1992; Murakmi et al., 1989; Wolff and Stern, 1991; Ward et al., 1992).

The porin proteins were initially co-isolated with lipopolysaccharides. Consequently, the porin proteins have been termed “endotoxin-associated proteins” (Bjornson et al., 1988). The meningococcal porins have been subdivided into three major classifications, which in antedated nomenclature were known as Class 1, 2, and 3 (Frasch et al., 1985(b)). Each meningococcal strain examined has contained one of the porB alleles for either a Class 2 porin gene or a Class 3 porin gene, but not both (Feavers et al., 1992; Murakani et al., 1989). Most meningococcal strains contain the porA gene (Class 1), but a few strains may not express the PorA protein due to phase variation. The data from the genes that have been thus far sequenced would suggest that all Neisseria porin proteins have at least 70% homology with each other, with some variations on a basic theme (Feavers et al., 1992). The porB (Class 2/3) genes are more closely related to each other than they are to the porA (Class 1) genes.

The development of immunogenic compositions targeted against serogroup B Neisseria meningitidis has concentrated on the use of outer membrane components, with a lead candidate being the PorA serosubtype antigen. Experimental immunogenic compositions with PorA protein have been tested in mice and immunogenic compositions of PorA-containing meningococcal outer membrane vesicles have been tested in human trials. These immunogenic compositions elicit a protective response against the homologous meningococcal strains, but show little or no heterologous protection. To produce an efficacious serogroup B immunogenic composition will require the use of multiple serosubtypes of the PorA protein to provide protection against the major disease causing strains. Based on epidemiological studies, prevention of greater than 65% of serogroup B disease in North America and Europe, will require at least a six valent and probably up to a nine valent PorA immunogenic composition.

Presently no immunogenic composition exists for Neisseria meningitidis serogroup B. A major impediment in the use of Neisseria porin proteins has been the inability to obtain sufficient quantities of purified porin proteins. For example, it has been observed that prolonged expression of Neisseria porin proteins in E. coli is lethal to the E. coli host cells (Koomey et al., 1991; Carbonetti and Sparling 1987; Carbonetti et al., 1988; U.S. Pat. No. 6,013,267 and U.S. Pat. No. 5,439,808). One approach to reduce toxicity of Neisseria porin proteins expressed in E. coli host cells has been the use of fusion constructs. Blake et al. reported the successful expression of a Neisseria meningitidis porin protein (i.e., a fusion protein) in an E. coli host cell by removing the meningococcal leader sequence and fusing the mature porin to the amino terminal 15 amino acids of the T7 φ10 capsid protein, “T7-tag” (U.S. Pat. No. 5,439,808).

It is observed in the present invention, that the recombinant expression in E. coli of five serosubtypes of PorA, (P1:5c,10, P1:5a,2c, P1:22,9, P1:22,14 and P1:21,16), occur only at low levels without the T7-tag fusion. It is contemplated that it will be advantageous to express PorA proteins as non-fusion proteins for use in the preparation of multivalent immunogenic compositions, wherein such a composition will comprise multiple PorA serosubtypes (e.g., a six valent, a seven valent, an eight valent or a nine valent PorA composition). Wherever possible, it is desirable to avoid introduction of extra amino acids in an immunogenic composition as it could introduce new epitopes, or alter folding of the PorA protein, either of which could affect PorA epitope presentation to the immune system.

### SUMMARY OF THE INVENTION

The present invention broadly relates to polynucleotide sequences encoding porin polypeptides of Neisseria. More particularly, the invention relates to newly identified nucleic acid sequence mutations in polynucleotides encoding PorA polypeptides of Neisseria meningitidis, wherein these sequence mutations result in increased expression levels of recombinant PorA polypeptides. In certain preferred embodiments, the polynucleotide encoding the PorA protein or polypeptide is cloned from a Neisseria meningitidis serogroup B isolate.

In a preferred embodiment, the invention is directed to a method for increasing the expression levels of a Neisseria PorA protein or polypeptide in a host cell comprising the steps of infecting, transfecting or transforming a host cell with an expression vector comprising a polynucleotide comprising a nucleotide sequence of SEQ ID NO:1, SEQ ID NO:3, SEQ ID NO:13, SEQ ID NO:15 or SEQ ID NO:24, wherein codon 18 is a codon other than an ATC; culturing the host cell under conditions suitable to produce the protein or polypeptide encoded by the polynucleotide of SEQ ID NO:1, SEQ ID NO:3, SEQ ID NO:13, SEQ ID NO:15 or SEQ ID NO:24; and recovering the protein or polypeptide from the culture.

In one preferred embodiment, the polynucleotide comprising the nucleotide sequence of SEQ ID NO:1 encodes a protein or polypeptide comprising an amino acid sequence of SEQ ID NO:2, wherein the amino acid at residue 18 is an amino acid other than an ATC encoded isoleucine residue. In another preferred embodiment, the polynucleotide comprising the nucleotide sequence of SEQ ID NO:3 encodes a protein or polypeptide comprising an amino acid sequence of SEQ ID NO:4, wherein the amino acid at residue 18 is an amino acid other than an ATC encoded isoleucine residue. In yet another preferred embodiment, the polynucleotide comprising the nucleotide sequence of SEQ ID NO:13 encodes a protein or polypeptide comprising an amino acid sequence of SEQ ID NO:14, wherein the amino acid at residue 18 is an amino acid other than an ATC encoded isoleucine residue. In still another preferred embodiment, the polynucleotide comprising the nucleotide sequence of SEQ ID NO:15 encodes a protein or polypeptide comprising an amino acid sequence of SEQ ID NO:16, wherein the amino acid at residue 18 is an amino acid other than an ATC encoded isoleucine residue. In yet another embodiment, the polynucleotide comprising the nucleotide sequence of SEQ ID NO:24 encodes a protein or polypeptide comprising an amino acid sequence of SEQ ID NO:25, wherein the amino acid at residue 18 is an amino acid other than an ATC encoded isoleucine residue. In certain preferred embodiments, codon 18 is a TAC codon. In one particular embodiment, the polynucleotide encoding the PorA protein or polypeptide is isolated from Neisseria meningitidis. In other embodiments, the polynucleotide is operatively linked to one or more gene expression regulatory elements. In a preferred embodiment, one of the regulatory elements is a promoter. In another embodiment, the vector is a plasmid, wherein a preferred plasmid vector is pET9a. In yet other embodiments, the host cell is a bacterial cell. In preferred embodiments, the host cell is E. coli. In preferred embodiments, the E. coli host cell is a strain comprising the DE3 lysogen. In another preferred embodiment, the E. coli is a strain selected from the group consisting of BLR(DE3)pLysS, BL21(DE3)pLysS, HMS174(DE3)pLysE and NovaBlue(DE3). In other embodiments of the invention, the protein or polypeptide expressed is at least about 30% of the total cellular protein concentration. In a more preferred embodiment, the protein or polypeptide expressed is at least about 50% of the total cellular protein concentration. In a most preferred embodiment, the protein or polypeptide expressed is at least about 75% of the total cellular protein concentration.

In another preferred embodiment of the invention, an isolated PorA protein or polypeptide is produced according to a method comprising infecting, transfecting or transforming a host cell with an expression vector comprising a polynucleotide comprising a nucleotide sequence of SEQ ID NO:1, SEQ ID NO:3, SEQ ID NO:13, SEQ ID NO:15 or SEQ ID NO:24, wherein codon 18 is a codon other than an ATC; culturing the host cell under conditions suitable to produce the protein or polypeptide encoded by the polynucleotide of SEQ ID NO:1, SEQ ID NO:3, SEQ ID NO:13, SEQ ID NO:15 or SEQ ID NO:24; and recovering the protein or polypeptide from the culture.

In still other embodiments the invention is directed to an isolated Neisseria meningitidis polynucleotide comprising a nucleotide sequence of SEQ ID NO:1, SEQ ID NO:3, SEQ ID NO:13, SEQ ID NO:15 or SEQ ID NO:24, wherein codon 18 is a codon other than an ATC codon. In certain preferred embodiments, codon 18 is a TAC codon.

In yet other embodiments, the invention is directed to an isolated Neisseria meningitidis PorA polypeptide or protein comprising an amino acid sequence of SEQ ID NO:2, SEQ ID NO:4, SEQ ID NO:14, SEQ ID NO:16 or SEQ ID NO:25, wherein the amino acid at residue 18 is an amino acid other than an ATC encoded isoleucine. In certain preferred embodiments, the amino acid at residue 18 is tyrosine.

In one preferred embodiment, the invention provides a recombinant expression vector comprising a polynucleotide having a nucleotide sequence of SEQ ID NO:1, SEQ ID NO:3, SEQ ID NO:13, SEQ ID NO:15 or SEQ ID NO:24, wherein codon 18 is a codon other than an ATC codon. In one particular embodiment, codon 18 is a TAC codon. In still other embodiments, the polynucleotide is selected from the group consisting of DNA, cDNA, RNA and mRNA. In one preferred embodiment, the vector is plasmid DNA. In yet other embodiments, the polynucleotide is operatively linked to one or more gene expression regulatory elements.

In certain embodiments, the invention is directed to a genetically engineered host cell transfected, transformed or infected with a recombinant expression vector comprising a polynucleotide having a nucleotide sequence of SEQ ID NO:1, SEQ ID NO:3, SEQ ID NO:13, SEQ ID NO:15 or SEQ ID NO:24, wherein codon 18 is a codon other than an ATC codon. In preferred embodiments, the host cell is a bacterial cell. In even more preferred embodiments, the bacterial host cell is E. coli. In certain embodiments, the E. coli host cell is a strain comprising the DE3 lysogen. In preferred embodiments, the bacterial host cell is an E. coli strain selected from the group consisting of BLR(DE3)pLysS, BL21(DE3)pLysS, HMS174(DE3)pLysE and NovaBlue(DE3). In a most preferred embodiment, the polynucleotide is expressed to produce the encoded polypeptide or protein.

The invention is directed in other embodiments to an immunogenic composition comprising a Neisseria meningitidis PorA polypeptide or protein having an amino acid sequence of SEQ ID NO:2, SEQ ID NO:4, SEQ ID NO:14, SEQ ID NO:16 or SEQ ID NO:25, wherein the amino acid at residue 18 is an amino acid other than an ATC encoded isoleucine. In preferred embodiments, the amino acid at residue 18 is tyrosine. In particular embodiments, the immunogenic composition further comprises one or more PorA polypeptides or proteins selected from the group consisting of SEQ ID NO:6, SEQ ID NO:8, SEQ ID NO:10, SEQ ID NO:12, SEQ ID NO:18 and SEQ ID NO:20. In yet other embodiments, the immunogenic composition further comprises one or more adjuvants.

In certain other embodiments, the invention is directed to an immunogenic composition comprising a Neisseria meningitidis PorA polypeptide or protein having an amino acid sequence of SEQ ID NO:2, SEQ ID NO:4, SEQ ID NO:14, SEQ ID NO:16 and SEQ ID NO:25, wherein the amino acid at residue 18 is an amino acid other than an ATC encoded isoleucine. In preferred embodiments, the amino acid at residue 18 is tyrosine. In particular embodiments, the immunogenic composition further comprises one or more PorA polypeptides or proteins selected from the group consisting of SEQ ID NO:6, SEQ ID NO:8, SEQ ID NO:10, SEQ ID NO:12, SEQ ID NO:18 and SEQ ID NO:20. In yet other embodiments, the immunogenic composition further comprises one or more adjuvants.

In certain other embodiments, the invention is directed to methods for identifying “endogenous” and/or “mature” Neisseria polynucleotide sequences encoding porin proteins or polypeptides which would be expressed at low levels in a host cell and methods for increasing the expression levels of said porin polypeptides or proteins in a host cell. An “endogenous” Neisseria polynucleotide sequence of the invention is a Neisseria sequence isolated from a naturally occurring Neisseria strain or a Neisseria sequence identified from a Neisseria sequence database, wherein the “endogenous” Neisseria polynucleotide sequence comprises nucleotides encoding a 5′ signal (or transport or leader) peptide sequence. In contrast, a “mature” Neisseria polynucleotide sequence lacks the nucleotides encoding the 5′ signal peptide sequence.

Thus, in certain embodiments, the invention is directed to a method for identifying Neisseria polynucleotide sequences encoding porin proteins or polypeptides which are expressed at low levels in a host cell, the method comprising obtaining a mature Neisseria polynucleotide sequence and determining the triplet sequence at codon 17, wherein an ATC at codon 17 indicates that the encoded porin protein or polypeptide is expressed at low levels in a host cell.

In another embodiment, the invention is directed to a method for identifying Neisseria polynucleotide sequences encoding porin proteins or polypeptides which are expressed at low levels in a host cell, the method comprising obtaining an endogenous Neisseria polynucleotide sequence; determining the 5′ signal sequence; hypothetically deleting the 5′ signal sequence and determining the triplet sequence at codon 17, wherein an ATC at codon 17 indicates that the encoded porin protein or polypeptide is expressed at low levels in a host cell.

In yet another embodiment, the invention is directed to a method for increasing the expression levels of a Neisseria porin polypeptide or protein in a host cell, the method comprising obtaining a mature Neisseria polynucleotide sequence; determining the triplet sequence at codon 17, wherein an ATC at codon 17 indicates that the encoded porin protein or polypeptide is expressed at low levels in a host cell and replacing codon 17 with a codon other than an ATC. In a preferred embodiment, a 5′-ATG codon is added to the sequence. In still another embodiment, the above method further comprises the steps of infecting, transfecting or transforming a host cell with an expression vector comprising the polynucleotide, culturing the host cell under conditions suitable to produce the encoded protein or polypeptide and recovering the protein or polypeptide from the culture. In a preferred embodiment, codon 17 is replaced with a TAC codon (or codon 18 is replaced with a TAC when a 5′-ATG codon is added).

In still other embodiments, the invention is directed to a method for increasing the expression levels of a Neisseria porin polypeptide or protein in a host cell, the method comprising obtaining an endogenous Neisseria polynucleotide sequence; determining the 5′ signal sequence; deleting the 5′ signal sequence; determining the triplet sequence at codon 17, wherein an ATC at codon 17 indicates that the encoded porin protein or polypeptide is expressed at low levels in a host cell and replacing codon 17 with a codon other than an ATC. In certain preferred embodiments, the method further comprises the step of adding a 5′-ATG codon to the sequence. In another preferred embodiment, the method further comprises the steps of infecting, transfecting or transforming a host cell with an expression vector comprising the polynucleotide; culturing the host cell under conditions suitable to produce the encoded protein or polypeptide; and recovering the protein or polypeptide from the culture.

In yet another embodiment, the invention is directed to a method for increasing the expression levels of a Neisseria porin polypeptide or protein in a host cell, the method comprising obtaining a mature Neisseria porA polynucleotide sequence; determining the triplet sequence at codon 17, wherein an ATC at codon 17 indicates that the encoded porin protein or polypeptide is expressed at low levels in a host cell and selecting an alternative Neisseria strain wherein codon 17 of the mature alternative porA sequence is a codon other than an ATC. In a preferred embodiment, the method further comprises the step of adding a 5′-ATG codon to the alternative Neisseria porA sequence. In another preferred embodiment, the method further comprises the steps of infecting, transfecting or transforming a host cell with an expression vector comprising the polynucleotide; culturing the host cell under conditions suitable to produce the encoded protein or polypeptide and recovering the protein or polypeptide from the culture. In one preferred embodiment, the porA sequence from the alternative strain has a TAC at codon 17 (or the alternative strain has a TAC at codon 18 when a 5′-ATG codon is added).

In another embodiment, the invention is directed to a method for increasing the expression levels of a Neisseria porin polypeptide or protein in a host cell, the method comprising obtaining an endogenous Neisseria porA polynucleotide sequence; determining the 5′ signal sequence; hypothetically deleting the 5′ signal sequence; determining the triplet sequence at codon 17, wherein an ATC at codon 17 indicates that the encoded porin protein or polypeptide is expressed at low levels in a host cell and selecting an alternative Neisseria strain, wherein codon 17 of the alternative Neisseria strain's mature porA sequence is a codon other than an ATC. In one preferred embodiment, the method further comprises the step of adding a 5′-ATG codon to the alternative Neisseria porA sequence. In another preferred embodiment, the method of further comprises the steps of infecting, transfecting or transforming a host cell with an expression vector comprising the polynucleotide; culturing the host cell under conditions suitable to produce the encoded protein or polypeptide and recovering the protein or polypeptide from the culture. In another preferred embodiment, the alternative strain has a TAC at codon 17 (or the alternative strain has a TAC at codon 18 when a 5′-ATG codon is added).

In certain embodiments, the invention is directed to isolated polynucleotides produced according to the methods of identifying “endogenous” and/or “mature” Neisseria polynucleotide sequences encoding porin proteins or polypeptides which would be expressed at low levels in a host cell and methods for increasing the expression levels of said porin polypeptides or proteins in a host cell. In still other embodiments, the invention is directed to isolated proteins or polypeptides produced according to the methods of identifying “endogenous” and/or “mature” Neisseria polynucleotide sequences encoding porin proteins or polypeptides which would be expressed at low levels in a host cell and methods for increasing the expression levels of said porin polypeptides or proteins in a host cell. In other embodiments, the invention is directed to recombinant expression vectors comprising a polynucleotide produced according to the methods of identifying “endogenous” and/or “mature” Neisseria polynucleotide sequences encoding porin proteins or polypeptides which would be expressed at low levels in a host cell and methods for increasing the expression levels of said porin polypeptides or proteins in a host cell. In further embodiments, the invention is directed to genetically engineered host cells transfected, transformed or infected with these recombinant vectors. In yet other embodiments, the invention is directed to immunogenic compositions comprising a polypeptide or protein produced according to the methods of identifying “endogenous” and/or “mature” Neisseria polynucleotide sequences encoding porin proteins or polypeptides which would be expressed at low levels in a host cell and methods for increasing the expression levels of said porin polypeptides or proteins in a host cell.

In one particular embodiment, the invention is directed to a method of immunizing against Neisseria comprising administering to a host an immunizing amount of an immunogenic composition comprising a polypeptide having an amino acid sequence of SEQ ID NO:2, or a fragment thereof and a pharmaceutically acceptable carrier, wherein the amino acid at residue 18 is an amino acid other than an ATC encoded isoleucine. In certain preferred embodiments, the amino acid at residue 18 is tyrosine.

In other embodiments, the invention is directed to a method of immunizing against Neisseria comprising administering to a host an immunizing amount of an immunogenic composition comprising a polypeptide having an amino acid sequence of SEQ ID NO:4, or a fragment thereof and a pharmaceutically acceptable carrier, wherein the amino acid at residue 18 is an amino acid other than an ATC encoded isoleucine. In certain preferred embodiments, the amino acid at residue 18 is tyrosine.

In still other embodiments, the invention is directed to method a of immunizing against Neisseria comprising administering to a host an immunizing amount of an immunogenic composition comprising a polypeptide having an amino acid sequence of SEQ ID NO:14, or a fragment thereof and a pharmaceutically acceptable carrier, wherein the amino acid at residue 18 is an amino acid other than an ATC encoded isoleucine. In particular embodiments, the amino acid at residue 18 is tyrosine.

In still another embodiment, the invention is directed to a method of immunizing against Neisseria comprising administering to a host an immunizing amount of an immunogenic composition comprising a polypeptide having an amino acid sequence of SEQ ID NO:16, or a fragment thereof and a pharmaceutically acceptable carrier, wherein the amino acid at residue 18 is an amino acid other than an ATC encoded isoleucine. In certain embodiments, the amino acid at residue 18 is tyrosine.

In still another embodiment, the invention is directed to a method of immunizing against Neisseria comprising administering to a host an immunizing amount of an immunogenic composition comprising a polypeptide having an amino acid sequence of SEQ ID NO:25, or a fragment thereof and a pharmaceutically acceptable carrier, wherein the amino acid at residue 18 is an amino acid other than an ATC encoded isoleucine. In certain embodiments, the amino acid at residue 18 is tyrosine.

In one embodiment, the invention is directed to a method of immunizing against Neisseria comprising administering to a host an immunizing amount of an immunogenic composition comprising a polypeptide having an amino acid sequence of SEQ ID NO:2 or a fragment thereof, a polypeptide having an amino acid sequence of SEQ ID NO:4 or a fragment thereof, a polypeptide having an amino acid sequence of SEQ ID NO:14 or a fragment thereof, a polypeptide having an amino acid sequence of SEQ ID NO:16 or a fragment thereof, a polypeptide having an amino acid sequence of SEQ ID NO:25 or a fragment thereof and a pharmaceutically acceptable carrier, wherein the amino acid at residue 18 of SEQ ID NO:2, SEQ ID NO:4, SEQ ID NO:14, SEQ ID NO:16 and SEQ ID NO:25 is an amino acid other than an ATC encoded isoleucine. In a preferred embodiment, the amino acid at residue 18 is tyrosine. In still other preferred embodiments, the method further comprises an adjuvant and/or one or more PorA polypeptides or proteins selected from the group consisting of SEQ ID NO:6, SEQ ID NO.8, SEQ ID NO:10, SEQ ID NO:12, SEQ ID NO:14, SEQ ID NO:16, SEQ ID NO:18 and SEQ ID NO:20.

Other features and advantages of the invention will be apparent from the following detailed description, from the preferred embodiments thereof, and from the claims.

### BRIEF DESCRIPTION OF THE DRAWINGS

FIG. 1 shows the T7 promoter region of the inducible expression plasmid pET9a. The T7 promoter sequence is comprised of nucleotides 615 to 631 and the ribosome binding site is comprised of nucleotides 560-565. The start codon (ATG) is in italics (nucleotides 549-551) and is part of a Ndel endonuclease restriction recognition site. The T7-Tag sequence spans nucleotides 519-548. The porA gene, with a 5′ Ndel restriction site, was cloned into the vector on a BgIII fragment at the BamHI restriction site. The PorA protein can be expressed as a T7-Tag amino terminal fusion or the Ndel fragment from nucleotide 506 to 551 can be deleted and the PorA protein can be expressed without the T7-Tag. The nucleotide numbering is based on the published pET9a DNA sequence from Novagen, Inc.

FIG. 2 is a porA 5′ nucleotide sequence alignment. Boxed residues differ from the consensus sequence.

FIG. 3 is a PorA polypeptide sequence alignment. Boxed residues differ from the consensus sequence.

FIG. 4 is a polyacrylamide protein gel (12% ) showing PorA expression in E. coli cell lines BLR(DE3)pLysS (FIG. 4A) or BL21 (DE3)pLysS (FIG. 4B) carrying the plasmid family pPX7303 (PorA subserotype P1:5a, 2c), with an ATC at codon 18. Each lane contains a whole cell lysate (WCL) of uninduced or induced expression of PorA from the T7 promoter contained on the plasmid pPX7303. Lane 1 shows the molecular weight markers (207, 123, 86, 44, 31, 18 and 7 kD). Lane 2 shows the PorA expression level from pPX7303 without IPTG induction. Lanes 3 and 4 show IPTG induction of PorA expression from either the T7-tag fusion protein (pPX7303-T7) or the mature PorA protein (pPX7303). Lane 5 contains the mutant plasmid, pPX7316, which changes the native porA codon 18 (ATC) to TAC. Note the enhanced level of PorA expression when the TAC is substituted for the ATC codon (lane 5).

### DETAILED DESCRIPTION OF THE INVENTION

The invention described hereinafter, addresses the need for Neisseria meningitidis immunogenic compositions that effectively cover most or all of the disease caused by serogroup B Neisseria meningitidis. Thus, it is highly desirable to prepare an immunogenic composition that protects against heterologous strains of Neisseria meningitidis serogroup B. A lead candidate in Neisseria meningitidis serogroup B immunogen development is the abundant and highly immunogenic outer membrane protein PorA. It is contemplated that an efficacious serogroup B immunogenic composition will require the use of multiple serosubtypes of the PorA protein and at least about a six to about a nine valent PorA immunogen to provide broad protection against endemic Neisseria meningitidis serogroup B strains. However, it is observed in the invention described hereinafter, that the recombinant expression of five serosubtypes of PorA occur only at low levels (e.g., serosubtypes P1:5a,2c (SEQ ID NO:3), P1:5c,10 (SEQ ID NO:l), P1:22,9 (SEQ ID NO:13), P1:21,16 (SEQ ID NO:15) and P1:22,14 (SEQ ID NO:24) when expressed as fusionless proteins.

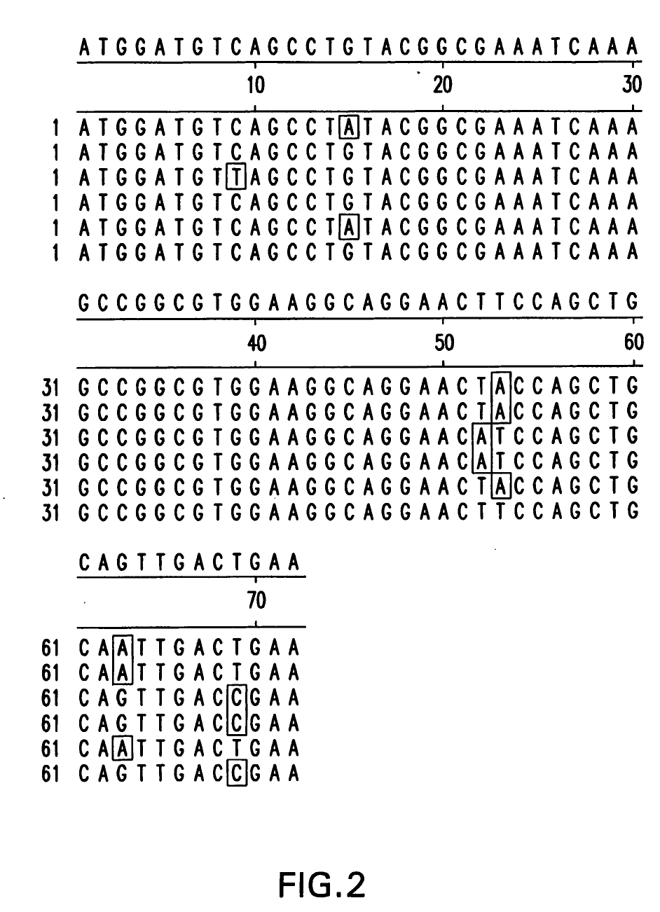

The present invention identifies novel nucleic acid sequence mutations in polynucleotides encoding PorA polypeptides of Neisseria meningitidis, wherein these sequence mutations result in increased expression levels of PorA polypeptides. Fifteen PorA serosubtype genes were cloned into a pET9a vector behind the highly active bacteriophage T7 promoter (Studier et al., 1990). The E. coli strain BLR(DE3)pLysS (Novagen, Inc.) was used as the host strain for recombinant expression from the pET9a/PorA plasmids. Ten of the fifteen serosubtype porA genes expressed well in this system. However, there were difficulties expressing five porA genes unless a T7 tag was fused to the amino terminus. Comparative analysis of the porA gene sequence (see Table 1) suggests the source of the expression problem is a difference in codon 18 of the porA gene in the plasmids expressing low-levels of PorA polypeptides. Those with a TAC (Tyr) codon at position 18 expressed at high levels, whereas those with an ATC (IIe) codon at position 18 expressed at low levels. An ATT (IIe) or TTC (Phe) codon at position 18 expressed at high levels. Site directed mutagenesis of the nucleotides encoding codon 18 converted the codon sequence from ATC to TAC, which matches the DNA sequence of the other highly expressing porA genes (FIG. 2 and Table 1). The altered (i.e., mutated at codon 18) porA genes from P1:5a,2c (i.e., SEQ ID NO:3 has a codon other than ATC at codon 18), P1:5c,10 (i.e., SEQ ID NO:1 has a codon other than ATC at codon 18), P1:22,9 (i.e., SEQ ID NO:13 has a codon other than ATC at codon 18) and P1:21,16 (i.e., SEQ ID NO:15 has a codon other than ATC at codon 18) now express high levels of their respective PorA protein, with PorA protein levels at 35-75% of total cellular protein.

TABLE 1

| Neisseria meningitidis | | Amino Acid |
|---|---|---|
| Serosubtype | Codon #18 | Residue #18 |
| P1:7,16 | TAC | Tyr |
| (SEQ ID NO: 5) | | |
| P1:7b,4 | TAC | Tyr |
| (SEQ ID N0: 7) | | |
| P1:7b,16 | TAC | Tyr |
| (SEQ ID NO: 17) | | |
| P1:22a,14 | TAC | Tyr |
| (SEQ ID NO: 11) | | |
| P1:5c,10 | ATC | Ile |
| (SEQ ID NO: 1) | | |
| P1:5a,2c | ATC | Ile |
| (SEQ ID NO: 3) | | |
| P1:21,16 | ATC | Ile |
| (SEQ ID NO: 15) | | |
| P1:22,9 | ATC | Ile |
| (SEQ ID NO: 13) | | |
| P1:22,14 | ATC | Ile |
| (SEQ ID NO: 24) | | |
| P1:18,25,6 | ATT | Ile |
| (SEQ ID NO: 19) | | |
| P1:19,15 | TTC | Phe |
| (SEQ ID NO: 9) | | |

As defined hereinafter, an “endogenous” Neisseria polynucleotide sequence encoding a secreted protein (or polypeptide) is a polynucleotide isolated or identified from a naturally occurring Neisseria strain and encodes a 5′ signal (or transport or leader) peptide sequence. Similarly, as defined hereinafter, an “endogenous” secreted Neisseria protein or polypeptide sequence is a Neisseria protein or polypeptide isolated or identified from a naturally occurring Neisseria strain and comprises a N-terminal signal (or transport or leader) peptide sequence. Specifically, for the PorA polypeptide, the signal sequence consists of nineteen amino acids, wherein a signal peptidase recognizes the N-terminal signal sequence via a proline turn at amino acid position −6, an alanine at amino acid position −3 and an alanine at amino acid position −1. The above numbering of the amino acids of the N-terminal sequence (i.e., −1 to −19) is used to distinguish the N-terminal signal sequence (i.e., the “endogenous” sequence) from the amino acids found in a “mature” sequence (i.e., lacking the N-terminal signal sequence). Thus, all amino acids with a negative number are comprised within the N-terminal signal sequence, wherein an amino acid designated −1 is next to the protease cleavage site and an amino acid designated −19 is located furthest upstream of the cleavage site (i.e., —19 is the N-terminal amino acid).

As defined hereinafter, a signal sequence generally exhibits three distinct features as follows: (1) a membrane spanning hydrophobic domain, (2) followed by a turn in the peptide sequence formed by either a proline or glycine at approximately amino acid position −6, relative to the cleavage site and (3) there is in general either an alanine, glycine or serine at both the −3 and −1 positions, relative to the cleavage site (Pugsley, 1993). Although different proteins have slight variations in signal sequence features, the majority of PorA sequences obtained to date have a nineteen amino acid signal sequence, with an alanine at amino acid positions −3 and −1. Computer programs such as SignalP, Sigcleave or SPScan can be used to predict the signal sequence of a protein and are well known in the art (Zagursky and Russell, 2001).

For the recombinant expression of endogenous Neisseria porin proteins or polypeptides (e.g., the PorA polypeptide) in a host cell, the 5′ nucleotides encoding the signal sequence are removed and a 5′ initiating methionine codon (ATG) is added in its place (i.e., replacing the 5′ signal sequence with a 5′ ATG codon). Thus, as defined hereinafter, a “mature” Neisseria polynucleotide sequence has the nucleotides encoding the signal sequence deleted from the endogenous Neisseria polynucleotide sequence. Similarly, a “mature +1” Neisseria polynucleotide sequence has the nucleotides encoding the signal sequence deleted from the endogenous Neisseria polynucleotide sequence, wherein the signal sequence has been substituted with a 5′ ATG codon. In addition, a “mature +1” Neisseria polynucleotide sequence of the invention may be represented as set forth in SEQ ID Nos: 1, 3, 5, 7, 9, 11, 13, 15, 17, 19 and 24, which include a 5′ methionine initiation codon (ATG) at position one in the nucleotide sequence.

As defined hereinafter, a “mature” Neisseria protein or polypeptide sequence of the invention is a protein or polypeptide sequence having its N-terminal signal peptide sequence removed from the endogenous amino acid sequence. Similarly, as defined hereinafter, a “mature +1” Neisseria protein or polypeptide sequence and/or a “recombinantly expressed” Neisseria protein or polypeptide of the invention is a protein or polypeptide sequence having its N-terminal signal peptide sequence removed from the endogenous amino acid sequence, wherein the signal peptide sequence has been replaced with a N-terminal methionine amino acid. In addition, a “mature +1” Neisseria protein or polypeptide of the invention may be represented as set forth in SEQ ID Nos: 2, 4, 6, 8, 10, 12, 14,16, 18, 20 and 25, which includes a N-terminal methionine residue at position one of the amino acid sequence.

As defined above, a “mature +1” Neisseria polynucleotide sequence has the nucleotides encoding the signal sequence deleted from the endogenous Neisseria polynucleotide sequence, wherein the signal sequence has been substituted with a 5′ ATG codon. Thus, codon 18 of the “mature +1” Neisseria nucleotide sequences set forth as SEQ ID Nos: 1, 3, 5, 7, 9, 11, 13, 15, 17, 19 and 24 is equivalent to codon 17 of a “mature” Neisseria sequence. Similarly, amino acid 18 of the “mature +1” protein or polypeptide as set forth in SEQ ID Nos: 2, 4, 6, 8, 10, 12, 14, 16, 18, 20 and 25 is equivalent to amino acid 17 of a “mature” protein or polypeptide. By way of a non-limiting example, if a particular codon or amino acid in a “mature” sequence equals (n), then the same codon or amino acid in a “mature +1” sequence equals (n+1) due to the addition of the ATG codon or the methionine amino acid, respectively.

Hereinafter, all references to a “Neisseria polynucleotide”, a “recombinant Neisseria polynucleotide” a “Neisseria polypeptide or protein” or a “recombinant Neisseria polypeptide or protein” are directed to “mature +1” Neisseria sequences, unless specifically referred to as an “endogenous” sequence or a “mature” sequence. In addition, hereinafter, all references to a “mutant” polynucleotide sequence, a “wildtype” polynucleotide sequence, a “mutant” polypeptide or protein sequence or a “wildtype” polypeptide or protein sequence, refer to a mature +1 Neisseria sequence unless specifically referred to as an “endogenous mutant” sequence, an “endogenous wildtype” sequence, a “mature mutant” sequence or a “mature wildtype” sequence.

Thus, as defined hereinafter, a Neisseria meningitidis strain 870227, serosubtype P1:5c,10 mutant porA polynucleotide sequence has a nucleic acid sequence of SEQ ID NO:1, wherein the wildtype ATC codon of SEQ ID NO:1 has been mutated to TAC at codon 18 and the encoded PorA protein or polypeptide has an amino acid sequence of SEQ ID NO:2, wherein the wildtype IIe amino acid residue 18 of SEQ ID NO:2 has been mutated to a Tyr amino acid residue. A Neisseria meningitidis strain NMB, serosubtype P1:5a,2c mutant porA polynucleotide has a nucleic acid sequence of SEQ ID NO:3, wherein the wildtype ATC codon of SEQ ID NO:3 has been mutated to TAC at codon 18 and the encoded PorA protein or polypeptide has an amino acid sequence of SEQ ID NO:4, wherein the wildtype IIe amino acid residue 18 of SEQ ID NO:4 has been mutated to a Tyr amino acid residue. A Neisseria meningitidis strain M982, serosubtype P1:22,9 mutant porA polynucleotide has a nucleic acid sequence of SEQ ID NO:13, wherein the wildtype ATC codon of SEQ ID NO:13 has been mutated to TAC at codon 18 and the encoded PorA protein or polypeptide has an amino acid sequence of SEQ ID NO:14, wherein the wildtype lie amino acid residue 18 of SEQ ID NO:14 has been mutated to a Tyr amino acid residue. A Neisseria meningitidis strain L4, serotype P1:21,16 mutant porA polynucleotide has a nucleic acid sequence of SEQ ID NO:15, wherein the wildtype ATC codon of SEQ ID NO:15 has been mutated to TAC at codon 18 and the encoded PorA protein or polypeptide has an amino acid sequence of SEQ ID NO:16, wherein the wildtype IIe amino acid residue 18 of SEQ ID NO:16 has been mutated to a Tyr amino acid residue. A Neisseria meningitidis strain M97 253462, serosubtype P1:22,14 mutant porA polynucleotide sequence has a nucleic acid sequence of SEQ ID NO:24, wherein the wildtype ATC codon of SEQ ID NO:24 has been mutated to TAC at codon 18 and the encoded PorA protein or polypeptide has an amino acid sequence of SEQ ID NO:25, wherein the wildtype IIe amino acid residue 18 of SEQ ID NO:25 has been mutated to a Tyr amino acid residue.

Further defined hereinafter is a Neisseria meningitidis strain H44/76, serosubtype P1:7,16 wildtype polynucleotide sequence of SEQ ID NO:5, a Neisseria meningitidis strain H44/76, serosubtype P1:7,16 wildtype polypeptide sequence of SEQ ID NO:6, a Neisseria meningitidis strain 880049, serosubtype P1 :7b,4 wildtype polynucleotide sequence of SEQ ID NO:7, a Neisseria meningitidis strain 880049, serosubtype P1:7b,4 wildtype polypeptide sequence of SEQ ID NO:8, a Neisseria meningitidis strain H355, serosubtype P1:19,15 polynucleotide sequence of SEQ ID NO:9, a Neisseria meningitidis strain H355, serosubtype P1:19,15 wildtype polypeptide sequence of SEQ ID NO:10, a Neisseria meningitidis strain 6557, serosubtype P1:22a,14 wildtype polynucleotide sequence of SEQ ID NO:11, a Neisseria meningitidis strain 6557, serosubtype P1 :22a,14 wildtype polypeptide sequence of SEQ ID NO:12, a Neisseria meningitidis strain M97 252097, serosubtype P1:7b,16 wildtype polynucleotide sequence SEQ ID NO:17, a Neisseria meningitidis strain M97 252097, serosubtype P1 :7b,16 wildtype polypeptide sequence of SEQ ID NO:18, a Neisseria meningitidis strain 6940, serosubtype P1:18,25,6 wildtype polynucleotide sequence of SEQ ID NO:19, and a Neisseria meningitidis strain 6940, serosubtype P1:18,25,6 wildtype polypeptide sequence of SEQ ID NO:20.

In addition, the examples described above are preferred in certain embodiments, but should not be construed as limiting. It is contemplated in the invention that replacing codon 18 with a codon other than an ATC results in the encoded PorA protein or polypeptide being expressed at high levels. For example, wildtype P1:18,25,6 (SEQ ID NO:19) has an ATT at codon 18, which encodes an isoleucine residue and P1:19,15 (SEQ ID NO:9) has a TTC at codon 18, which encodes a phenylalanine residue, both express well as fusion-less proteins. Thus, in addition to an ATC to TAC substitution at codon 18, other substitutions at codon 18 (e.g., ATC to TTC or ATC to ATT) are contemplated, as long as the encoded porin protein or polypeptide is being expressed at high levels.

A. Neisseria Polynucleotides Encoding PorA Polypeptides

Isolated and purified Neisseria polynucleotides of the present invention are contemplated for use in the production of Neisseria polypeptides. More specifically, in certain embodiments, the polynucleotides encode Neisseria porin polypeptides, particularly PorA polypeptides from Neisseria meningitidis. Thus, in one aspect, the present invention provides isolated and purified polynucleotides that encode Neisseria meningitidis serogroup B PorA polypeptides, wherein a polynucleotide comprising an ATC at codon 18 is mutated to a TAC codon, resulting in increased PorA protein expression levels. It is contemplated in particular embodiments that increased PorA protein expression levels facilitate the preparation of multivalent immunogenic compositions, e.g., a six valent, a seven valent, an eight valent or a nine valent PorA composition which protects against Neisseria meningitidis infection. In other embodiments, the invention provides methods for identifying “endogenous” and/or “mature” Neisseria polynucleotide sequences that encode PorA polypeptides which would be expressed at low levels in a host cell and methods for increasing the expression levels of said polypeptides or proteins in a host cell.

Further contemplated in the invention is the identification of Neisseria polynucleotides which express porin proteins at low levels, wherein low expression levels are associated with an ATC at codon 17 of a mature sequence or at codon 18 of a mature +1 sequence. As described above, mutation of the ATC codon to TAC codon increases the expression level of the encoded Neisseria porin protein. The increased expression levels of such porin proteins will further facilitate the isolation and purification of sufficient quantities to be tested and/or used as immunogenic compositions to protect against Neisseria infection, particularly Neisseria meningitidis infection.

In particular embodiments, a polynucleotide of the present invention is a DNA molecule, wherein the DNA may be chromosomal DNA, plasmid DNA or cDNA. In a preferred embodiment, a polynucleotide of the present invention is a recombinant polynucleotide, which encodes a Neisseria meningitidis PorA polypeptide. In another embodiment, an isolated and purified polynucleotide encoding a PorA polypeptide comprises a nucleotide sequence of SEQ ID NO:1, SEQ ID NO:3, SEQ ID NO:13, SEQ ID NO:15 or SEQ ID NO:24, wherein codon 18 is a codon other than an ATC. In another preferred embodiment, the polynucleotide is comprised in a plasmid vector and expressed in a prokaryotic host cell.

As used hereinafter, the term “polynucleotide” means a sequence of nucleotides connected by phosphodiester linkages. Polynucleotides are presented hereinafter in the 5′ to the 3′ direction. A polynucleotide of the present invention comprises from about 40 to about several hundred thousand base pairs. Preferably, a polynucleotide comprises from about 10 to about 3,000 base pairs. Preferred lengths of particular polynucleotide are set forth hereinafter.

A polynucleotide of the present invention is a deoxyribonucleic acid (DNA) molecule, a ribonucleic acid (RNA) molecule, or analogs of the DNA or RNA generated using nucleotide analogs. The nucleic acid molecule is single-stranded or double-stranded, but preferably is double-stranded DNA. Where a polynucleotide is a DNA molecule, that molecule is a gene, a cDNA molecule or a genomic DNA molecule. Nucleotide bases are indicated hereinafter by a single letter code: adenine (A), guanine (G), thymine (T), cytosine (C), inosine (I) and uracil (U).

“Isolated” means altered “by the hand of man” from the natural state. An “isolated” composition or substance is one that has been changed or removed from its original environment, or both. For example, a polynucleotide or a polypeptide naturally present in a living animal is not “isolated,” but the same polynucleotide or polypeptide separated from the coexisting materials of its natural state is “isolated,” as the term is employed hereinafter.

Preferably, an “isolated” polynucleotide is free of sequences which naturally flank the nucleic acid (i.e., sequences located at the 5′ and 3′ ends of the nucleic acid) in the genomic DNA of the organism from which the nucleic acid is derived. For example, in various embodiments, the isolated Neisseria meningitidis nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. However, the Neisseria meningitidis nucleic acid molecule can be fused to other protein encoding or regulatory sequences and still be considered isolated.

Neisseria meningitidis polynucleotides of the present invention are obtained, using standard cloning and screening techniques, from a cDNA library derived from mRNA. Polynucleotides of the invention also are obtained from natural sources such as genomic DNA libraries (e.g., a Neisseria meningitidis library) or are synthesized using well known and commercially available techniques.

The invention further encompasses nucleic acid molecules that differ from the nucleotide sequences shown in SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19 and SEQ ID NO:24 (and fragments thereof) due to degeneracy of the genetic code and thus encode the same Neisseria meningitidis polypeptide as that encoded by the nucleotide sequence shown SEQ ID NO:1, SEQ ID NO:3, SEQ ID NO:5, SEQ ID NO:7, SEQ ID NO:9, SEQ ID NO:l1, SEQ ID NO:13, SEQ ID NO:15, SEQ ID NO:17, SEQ ID NO:19 and SEQ ID NO:24.

Moreover, the polynucleotide of the invention can comprise only a fragment of the coding region of a Neisseria meningitidis polynucleotide or gene, such as a fragment of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19 or SEQ ID NO:24. In particular embodiments, it is desirable that such a fragment encode an antigenic PorA polypeptide fragment.

Thus, in certain embodiments, the polynucleotide sequence information provided by the present invention allows for the preparation of relatively short DNA (or RNA) oligonucleotide sequences having the ability to specifically hybridize to gene sequences of the selected polynucleotides disclosed hereinafter. The term “oligonucleotide” as used hereinafter is defined as a molecule comprised of two or more deoxyribonucleotides or ribonucleotides, usually more than three (3), and typically more than ten (10) and up to one hundred (100) or more (although preferably between twenty and thirty). The exact size will depend on many factors, which in turn depends on the ultimate function or use of the oligonucleotide. Thus, in particular embodiments of the invention, nucleic acid probes of an appropriate length are prepared based on a consideration of a selected nucleotide sequence. The ability of such nucleic acid probes to specifically hybridize to a polynucleotide encoding a Neisseria meningitidis polypeptide lends them particular utility in a variety of embodiments. Most importantly, the probe can be used in a variety of assays for detecting the presence of complementary sequences in a given sample.

To provide certain of the advantages in accordance with the present invention, a preferred nucleic acid sequence employed for hybridization studies or assays includes probe molecules that are complementary to at least a 10 to 70 or so long nucleotide stretch of a polynucleotide that encodes a Neisseria meningitidis polypeptide, such as that shown in SEQ ID NO:2, SEQ ID NO: 4, SEQ ID NO:14, SEQ ID NO:16 or SEQ ID NO:25. A size of at least 10 nucleotides in length helps to ensure that the fragment will be of sufficient length to form a duplex molecule that is both stable and selective. Molecules having complementary sequences over stretches greater than 10 bases in length are generally preferred, though, in order to increase stability and selectivity of the hybrid, and thereby improve the quality and degree of specific hybrid molecules obtained. One will generally prefer to design nucleic acid molecules having gene-complementary stretches of 25 to 40 nucleotides, 55 to 70 nucleotides, or even longer where desired. Such fragments are readily prepared, for example, by directly synthesizing the fragment by chemical means, by application of nucleic acid reproduction technology, such as the PCR technology of (U.S. Pat. No. 4,683,202, incorporated hereinafter by reference in its entirety) or by excising selected DNA fragments from recombinant plasmids containing appropriate inserts and suitable restriction enzyme sites.

In another aspect, the present invention contemplates an isolated and purified polynucleotide comprising a nucleotide sequence that is identical or complementary to a segment of at least 10 contiguous bases of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19 or SEQ ID NO:24, wherein the polynucleotide hybridizes to a polynucleotide that encodes a Neisseria meningitidis polypeptide. Preferably, the isolated and purified polynucleotide comprises a base sequence that is identical or complementary to a segment of at least 25 to 70 contiguous bases of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19 or SEQ ID NO:24. For example, the polynucleotide of the invention can comprise a segment of bases identical or complementary to 40 or 55 contiguous bases of the disclosed nucleotide sequences.

Accordingly, a polynucleotide probe molecule of the invention can be used for its ability to selectively form duplex molecules with complementary stretches of the gene. Depending on the application envisioned, one will desire to employ varying conditions of hybridization stringency to achieve varying degree of selectivity of the probe toward the target sequence (see Table 2). For applications requiring a high degree of selectivity, one will typically desire to employ relatively stringent conditions to form the hybrids. For some applications, for example, where one desires to prepare mutants employing a mutant primer strand hybridized to an underlying template or where one seeks to isolate a Neisseria meningitidis homologous polypeptide coding sequence from other cells, functional equivalents, or the like, less stringent hybridization conditions are typically needed to allow formation of the heteroduplex (see Table 2). Cross-hybridizing species can thereby be readily identified as positively hybridizing signals with respect to control hybridizations. In any case, it is generally appreciated that conditions can be rendered more stringent by the addition of increasing amounts of formamide, which serves to destabilize the hybrid duplex in the same manner as increased temperature. Thus, hybridization conditions can be readily manipulated, and thus will generally be a method of choice depending on the desired results.

The present invention also includes polynucleotides capable of hybridizing under reduced stringency conditions, more preferably stringent conditions, and most preferably highly stringent conditions, to polynucleotides described hereinafter. Examples of stringency conditions are shown in Table 3 below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 2
HYBRIDIZATION STRINGENCY CONDITIONS

| | Poly- | Hybrid | Hybridization | Wash |
|---|---|---|---|---|
| Stringency | nucleotide | Length | Temperature | Temperature |
| Condition | Hybrid | (bp)^{I} | and Buffer^{H} | and Buffer^{H} |
| A | DNA:DNA | >50 | 65° C.; 1 × SSC -or- | 65° C.; |
| | | | 42° C.; 1 × SSC, 50% | 0.3 × SSC |
| | | | formamide | |
| B | DNA:DNA | <50 | T_{B}; 1 × SSC | T_{B}; 1 × SSC |
| C | DNA:RNA | >50 | 67° C.; 1 × SSC -or- | 67° C.; |
| | | | 45° C.; 1 × SSC, 50% | 0.3 × SSC |
| | | | formamide | |
| D | DNA:RNA | <50 | T_{D}; 1 × SSC | T_{D}; 1 × SSC |
| E | RNA:RNA | >50 | 70° C.; 1 × SSC -or- | 70° C.; |
| | | | 50° C.; 1 × SSC, 50% | 0.3 × SSC |
| | | | formamide | |
| F | RNA:RNA | <50 | T_{F}; 1 × SSC | T_{F}; 1 × SSC |
| G | DNA:DNA | >50 | 65° C.; 4 × SSC -or- | 65° C.; |
| | | | 42° C.; 4 × SSC, 50% | 1 × SSC |
| | | | formamide | |
| H | DNA:DNA | <50 | T_{H}; 4 × SSC | T_{H}; 4 × SSC |
| I | DNA:RNA | >50 | 67° C.; 4 × SSC -or- | 67° C.; |
| | | | 45° C.; 4 × SSC, 50% | 1 × SSC |
| | | | formamide | |
| J | DNA:RNA | <50 | T_{J}; 4 × SSC | T_{J}; 4 × SSC |
| K | RNA:RNA | >50 | 70° C.; 4 × SSC -or- | 67° C.; |
| | | | 50° C.; 4 × SSC, 50% | 1 × SSC |
| | | | formamide | |
| L | RNA:RNA | <50 | T_{L}; 2 × SSC | T_{L}; 2 × SSC |
| M | DNA:DNA | >50 | 50° C.; 4 × SSC -or- | 50° C.; |
| | | | 40° C.; 6 × SSC, 50% | 2 × SSC |
| | | | formamide | |
| N | DNA:DNA | <50 | T_{N}; 6 × SSC | T_{N}; 6 × SSC |
| O | DNA:RNA | >50 | 55° C.; 4 × SSC -or- | 55° C.; |
| | | | 42° C.; 6 × SSC, 50% | 2 × SSC |
| | | | formamide | |
| P | DNA:RNA | <50 | T_{P}; 6 × SSC | T_{P}; 6 × SSC |
| Q | RNA:RNA | >50 | 60° C.; 4 × SSC -or- | 60° C.; |
| | | | 45° C.; 6 × SSC, 50% | 2 × SSC |
| | | | formamide | |
| R | RNA:RNA | <50 | T_{R}; 4 × SSC | T_{R}; 4 × SSC |
| (bp)^{I}: The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length is determined by aligning the sequences of the polynucleotides and identifying | | | | |
| # the region or regions of optimal sequence complementarity. | | | | |
| Buffer^{H}: SSPE (1 × SSPE is 0.15M NaCl, 10 mM NaH₂PO₄, and 1.25 mM EDTA, pH 7.4) can be substituted for SSC (1 × SSC is 0.15M NaCl and 15 mM sodium citrate) in the hybridization and wash buffers; washes are performed for 15 minutes after hybridization is complete. | | | | |
| T_{B} through T_{R}: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). | | | | |
| # For hybrids between 18 and 49 base pairs in length, Tₘ(° C.) = 81.5 + 16.6(log₁₀[Na⁺]) + 0.41(% G + C) − (600/N), where N is the number of bases in the hybrid, and [Na⁺] is the concentration of sodium ions in the hybridization buffer ([Na⁺] for 1 × SSC = 0.165 M). | | | | |

Additional examples of stringency conditions for polynucleotide hybridization are provided in Sambrook et al., 1989, Molecular Cloning: A Laboratory Manual, Cold Spring Harbor Laboratory Press, Cold Spring Harbor, N.Y., chapters 9 and 11, and Ausubel et al., 1995, Current Protocols in Molecular Biology, eds., John Wiley & Sons, Inc., sections 2.10 and 6.3-6.4, incorporated hereinafter by reference.

B. Neisseria Meningitidis PorA Polypeptides

Isolated and purified Neisseria porin polypeptides or proteins of the present invention are contemplated for use in the production of immunogenic compositions for immunizing a host against Neisseria infection. In particular embodiments, an isolated porin polypeptide or protein is the PorA polypeptide from Neisseria meningitidis. In certain embodiments, the invention is directed to methods for increasing expression levels of recombinant Neisseria meningitidis PorA polypeptides or proteins. In certain preferred embodiments, the PorA polypeptide or protein has an amino acid sequence of SEQ ID NO: 2, SEQ ID NO: 4, SEQ ID NO:14, SEQ ID NO:16 or SEQ ID NO:25, wherein the amino acid at residue 18 is a Tyr in SEQ ID NO: 2, SEQ ID NO: 4, SEQ ID NO:14, SEQ ID NO:16 and SEQ ID NO:25. In particular embodiments, the present invention provides isolated and purified Neisseria meningitidis polypeptides. Preferably, a Neisseria meningitidis polypeptide of the invention is a recombinant polypeptide. In certain embodiments, a Neisseria meningitidis polypeptide of the present invention is a PorA polypeptide comprising an amino acid sequence of SEQ ID NO:2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20 or SEQ ID NO:25, a biological equivalent thereof, or a fragment thereof.

In certain other embodiments, the invention provides “mature” and/or “endogenous” Neisseria meningitidis polynucleotide sequences which have been identified as encoding porin polypeptide sequences which would be expressed at low levels in a host cell (e.g., see Example 3). In certain preferred embodiments, the invention provides methods for increasing (e.g., mutating codon 17 of a “mature” sequence) the expression levels of said porin polypeptides in a host cell. Thus, in particular embodiments, the invention provides Neisseria meningitidis polynucleotides and polypeptides obtained from the methods of the present invention.

A biological equivalent or variant of a Neisseria meningitidis polypeptide according to the present invention encompasses 1) a polypeptide isolated from Neisseria meningitidis and 2) a polypeptide that contains substantial homology to a Neisseria meningitidis polypeptide.

Biological equivalents or variants of Neisseria meningitidis include both functional and non-functional Neisseria meningitidis polypeptides. Functional biological equivalents or variants are naturally occurring amino acid sequence variants of a Neisseria meningitidis polypeptide that maintains the ability to elicit an immunological or antigenic response in a subject. Functional variants will typically contain only conservative substitution of one or more amino acids of, e.g., SEQ ID NO:2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20 or SEQ ID NO:25, or substitution, deletion or insertion of non-critical residues in non-critical regions (i.e., epitope regions) of the polypeptide.

Modifications and changes can be made in the structure of a polypeptide of the present invention and still obtain a molecule having Neisseria meningitidis antigenicity. For example, certain amino acids are substituted for other amino acids in a sequence without appreciable loss of antigenicity. Because it is the interactive capacity and nature of a polypeptide that defines that polypeptide's biological functional activity, certain amino acid sequence substitutions can be made in a polypeptide sequence (or, of course, its underlying DNA coding sequence) and nevertheless obtain a polypeptide with like properties.

In making such changes, the hydropathic index of amino acids can be considered. The importance of the hydropathic amino acid index in conferring interactive biologic function on a polypeptide is generally understood in the art (Kyte & Doolittle, 1982). It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid residue determines the secondary and tertiary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within +/−2 is preferred, those that are within +/−1 are particularly preferred, and those within +/−0.5 are even more particularly preferred.

Substitution of like amino acids can also be made on the basis of hydrophilicity, particularly where the biological functional equivalent polypeptide or peptide thereby created is intended for use in immunological embodiments. U.S. Pat. No. 4,554,101, incorporated hereinafter by reference, states that the greatest local average hydrophilicity of a polypeptide, as governed by the hydrophilicity of its adjacent amino acids, correlates with its immunogenicity and antigenicity, i.e. with a biological property of the polypeptide.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those that are within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine (See Table 3, below). The present invention thus contemplates functional or biological equivalents of a Neisseria meningitidis polypeptide as set forth above.

TABLE 3
AMINO ACID SUBSTITUTIONS

| Original | Exemplary Residue |
|---|---|
| Residue | Substitution |
| Ala | Gly; Ser |
| Arg | Lys |
| Asn | Gln; His |
| Asp | Glu |
| Cys | Ser |
| Gln | Asn |
| Glu | Asp |
| Gly | Ala |
| His | Asn; Gln |
| Ile | Leu; Val |
| Leu | Ile; Val |
| Lys | Arg |
| Met | Leu; Tyr |
| Ser | Thr |
| Thr | Ser |
| Trp | Tyr |
| Tyr | Trp; Phe |
| Val | Ile; Leu |

A Neisseria meningitidis polypeptide or polypeptide antigen of the present invention is understood to be any Neisseria meningitidis polypeptide comprising substantial sequence similarity, structural similarity and/or functional similarity to a Neisseria meningitidis polypeptide comprising the amino acid sequence of one of SEQ ID NO:2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20 or SEQ ID NO:25.

It is contemplated in the present invention, that a Neisseria meningitidis polypeptide may advantageously be cleaved into fragments for use in further structural or functional analysis, or in the generation of reagents such as Neisseria meningitidis related polypeptides, PorA antigenic fragments and Neisseria meningitidis specific antibodies. This can be accomplished by treating purified or unpurified Neisseria meningitidis polypeptides with a peptidase such as endoproteinase glu-C (Roche Diagnostics Corp., Basel, Switzerland). Treatment with CNBr is another method by which peptide fragments may be produced from natural Neisseria meningitidis polypeptides. Recombinant techniques also can be used to produce specific fragments of a Neisseria meningitidis polypeptide.

A fragment is a polypeptide having an amino acid sequence that entirely is the same as part, but not all, of the amino acid sequence. The fragment can comprise, for example, at least 7 or more (e.g., 8, 10, 12, 14, 16, 18, 20, or more) contiguous amino acids of an amino acid sequence of SEQ ID NO:2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20 or SEQ ID NO:25. Fragments may be “freestanding” or comprised within a larger polypeptide of which they form a part or region, most preferably as a single, continuous region. In one embodiment, the fragments include at least one epitope of the mature polypeptide sequence.

In certain embodiments of the invention, it may be useful to make a PorA fusion protein. As defined herein, a “fusion protein” refers to a protein or polypeptide encoded by two, often unrelated (i.e., heterologous), fused genes or fragments thereof.

C. Vectors, Host Cells and Recombinant Neisseria Meningitidis Polypeptides

In a preferred embodiment, the present invention provides expression vectors comprising polynucleotides that encode Neisseria meningitidis polypeptides. Preferably, the expression vectors of the invention comprise polynucleotides that encode Neisseria meningitidis PorA polypeptides comprising the amino acid sequence of one of SEQ ID NO:2 (wherein the amino acid at residue 18 is a Tyr), SEQ ID NO: 4 (wherein the amino acid at residue 18 is a Tyr), SEQ ID NO: 6, SEQ ID NO: 8, SEQ ID NO: 10, SEQ ID NO: 12, SEQ ID NO: 14 (wherein the amino acid at residue 18 is a Tyr), SEQ ID NO: 16 (wherein the amino acid at residue 18 is a Tyr), SEQ ID NO: 18, SEQ ID NO: 20 or SEQ ID NO:25 (wherein the amino acid at residue 18 is a Tyr). More preferably, the expression vectors of the invention comprise a polynucleotide comprising the nucleotide base sequence of SEQ ID NO:1 (wherein codon 18 is TAC), SEQ ID NO: 3 (wherein codon 18 is TAC), SEQ ID NO: 5, SEQ ID NO: 7, SEQ ID NO: 9, SEQ ID NO: 11, SEQ ID NO: 13 (wherein codon 18 is TAC), SEQ ID NO: 15 (wherein codon 18 is TAC), SEQ ID NO: 17, SEQ ID NO: 19 or SEQ ID NO:24 (wherein codon 18 is TAC). In certain embodiments the expression vectors of the invention comprise a polynucleotide operatively linked to a prokaryotic promoter.

Expression of proteins in prokaryotes is most often carried out in E. coli with vectors containing constitutive or inducible promoters. In preferred embodiments, the PorA proteins are expressed as non-fusion proteins.

Examples of suitable inducible non-fusion E. coli expression vectors include pTrc (Amann et al., 1988) and pET derivatives (Studier et al., 1990) pBAD (Guzman et al., 1995), pRSET (Invitrogen Life Technologies), LITMUS (Evans et al. 1995), pMAL (Zagursky et al, 1984), pLEX (LaVallie et al., 1992), pCX-TOPO (Invitrogen Life Technologies).

One strategy to maximize recombinant protein expression in E. coli is to express the protein in a host bacterium with an impaired capacity to proteolytically cleave the recombinant protein. Another strategy is to alter the nucleic acid sequence of the nucleic acid to be inserted into an expression vector so that the individual codons for each amino acid are those preferentially utilized in E. coli. Such alteration of nucleic acid sequences of the invention is carried out by standard DNA mutagenesis or synthesis techniques (See Section A).

In other embodiments, a nucleic acid of the invention is expressed in mammalian cells using a mammalian expression vector. Examples of mammalian expression vectors include pCDM8 (Seed, 1987), and pMT2PC (Kaufman et al., 1987). When used in mammalian cells, the expression vector's control functions are often provided by viral regulatory elements

Another aspect of the invention pertains to host cells into which a recombinant expression vector of the invention has been introduced. The terms “host cell” and “recombinant host cell” are used interchangeably hereinafter. It is understood that such terms refer not only to the particular subject cell, but to the progeny or potential progeny of such a cell. Because certain modifications may occur in succeeding generations due to either mutation or environmental influences, such progeny may not, in fact, be identical to the parent cell, but are still included within the scope of the term as used hereinafter. A host cell can be any prokaryotic or eukaryotic cell. For example, a Neisseria meningitidis polypeptide can be expressed in bacterial cells such as E. coli, yeast or mammalian cells (such as Chinese hamster ovary cells (CHO), NIH 3T3, PERC.6, NSO, VERO, chick embryo fibroblasts, BHK cells or COS cells). Other suitable host cells are known to those skilled in the art.

Vector DNA can be introduced into prokaryotic or eukaryotic cells via conventional transformation, infection or transfection techniques. As used hereinafter, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, ultrasound or electroporation. Suitable methods for transforming or transfecting host cells can be found in Sambrook, et al. (“Molecular Cloning: A Laboratory Manual” 2nd, ed, Cold Spring Harbor Laboratory, Cold Spring Harbor Laboratory Press, Cold Spring Harbor, N.Y., 1989), and other laboratory manuals.

A host cell of the invention, such as a prokaryotic or eukaryotic host cell in culture, can be used to produce (i.e., express) a Neisseria meningitidis polypeptide. Accordingly, the invention further provides methods for producing a Neisseria meningitidis polypeptide using the host cells of the invention. In one embodiment, the method comprises culturing the host cell of invention (into which a recombinant expression vector encoding a Neisseria meningitidis polypeptide has been introduced) in a suitable medium until the Neisseria meningitidis polypeptide is produced. In another embodiment, the method further comprises isolating the Neisseria meningitidis polypeptide from the medium or the host cell.

As used hereinafter, a promoter is a region of a DNA molecule typically within about 100 nucleotide pairs in front of (upstream of) the point at which transcription begins (i.e., a transcription start site). That region typically contains several types of DNA sequence elements that are located in similar relative positions in different genes. As used hereinafter, the term “promoter” includes what is referred to in the art as an upstream promoter region and a promoter region.

Another type of discrete transcription regulatory sequence element is an enhancer. An enhancer provides specificity of time, location and expression level for a particular encoding region (e.g., gene). A major function of an enhancer is to increase the level of transcription of a coding sequence in a cell that contains one or more transcription factors that bind to that enhancer. Unlike a promoter, an enhancer can function when located at variable distances from transcription start sites so long as a promoter is present.

As used hereinafter, the phrase “enhancer-promoter” means a composite unit that contains both enhancer and promoter elements. An enhancer-promoter is operatively linked to a coding sequence that encodes at least one gene product. As used hereinafter, the phrase “operatively linked” means that an enhancer-promoter is connected to a coding sequence in such a way that the transcription of that coding sequence is controlled and regulated by that enhancer-promoter. Means for operatively linking an enhancer-promoter to a coding sequence are well known in the art. As is also well known in the art, the precise orientation and location relative to a coding sequence whose transcription is controlled, is dependent inter alia upon the specific nature of the enhancer-promoter. Thus, a TATA box minimal promoter is typically located from about 25 to about 30 base pairs upstream of a transcription initiation site and an upstream promoter element is typically located from about 100 to about 200 base pairs upstream of a transcription initiation site. In contrast, an enhancer can be located downstream from the initiation site and can be at a considerable distance from that site.

An enhancer-promoter used in a vector construct of the present invention is any enhancer-promoter that drives expression in a cell to be transfected. By employing an enhancer-promoter with well-known properties, the level and pattern of gene product expression can be optimized.

A coding sequence of an expression vector is operatively linked to a transcription termination region. RNA polymerase transcribes an encoding DNA sequence, where typically the DNA sequences located downstream of the polyadenylation site serve to terminate transcription. Those DNA sequences are referred to hereinafter as transcription-termination regions. Those regions are required for efficient polyadenylation of transcribed messenger RNA (mRNA). Transcription-termination regions are well known in the art. A preferred transcription-termination region used in an adenovirus vector construct of the present invention comprises a polyadenylation signal of SV40 or the protamine gene.

An expression vector comprises a polynucleotide that encodes a Neisseria meningitidis polypeptide. Such a polypeptide is meant to include a sequence of nucleotide bases encoding a Neisseria meningitidis polypeptide sufficient in length to distinguish the segment from a polynucleotide segment encoding a non Neisseria meningitidis polypeptide. A polypeptide of the invention can also encode biologically functional polypeptides or peptides which have variant amino acid sequences, such as with changes selected based on considerations such as the relative hydropathic score of the amino acids being exchanged. These variant sequences are those isolated from natural sources or induced in the sequences disclosed hereinafter using a mutagenic procedure such as site-directed mutagenesis.

Preferably, the expression vectors of the present invention comprise polynucleotides that encode polypeptides comprising the amino acid residue sequence of SEQ ID NO:2 (wherein the amino acid at residue 18 is a Tyr), SEQ ID NO: 4 (wherein the amino acid at residue 18 is a Tyr), SEQ ID NO: 6, SEQ ID NO: 8, SEQ ID NO: 10, SEQ ID NO: 12, SEQ ID NO: 14, SEQ ID NO: 16, SEQ ID NO: 18 SEQ ID NO: 20 or SEQ ID NO:25 (wherein the amino acid at residue 18 is a Tyr). An expression vector can include a Neisseria meningitidis polypeptide coding region itself of any of the Neisseria meningitidis polypeptides noted above or it can contain coding regions bearing selected alterations or modifications in the basic coding region of such a Neisseria meningitidis polypeptide. Alternatively, such vectors or fragments can code larger polypeptides or polypeptides which nevertheless include the basic coding region. In any event, it should be appreciated that due to codon redundancy as well as biological functional equivalence, this aspect of the invention is not limited to the particular DNA molecules corresponding to the polypeptide sequences noted above.

A DNA molecule of the present invention can be incorporated into a vector by a number of techniques that are well known in the art. For instance, the pET vectors have been demonstrated to be of particular value.

An expression vector of the present invention is useful both as a means for preparing quantities of the Neisseria meningitidis polypeptide-encoding DNA itself, and as a means for preparing the encoded polypeptide and peptides. It is contemplated that where Neisseria meningitidis polypeptides of the invention are made by recombinant means, one can employ prokaryotic expression vectors as shuttle systems. In another aspect, the recombinant host cells of the present invention are prokaryotic host cells. Preferably, the recombinant host cells of the invention are bacterial cells of the BL21(DE3) strain of Escherichia coli. In general, prokaryotes are preferred for the initial cloning of DNA sequences and constructing the vectors useful in the invention. For example, E. coli K12 strains can be particularly useful. Other microbial strains that can be used include E. coli B, and E. coliₓ1976 (ATCC No. 31537). These examples are, of course, intended to be illustrative rather than limiting.

In preferred embodiments, the recombinant host cells of the present invention are prokaryotic host cells. Preferably, the recombinant host cells of the invention are bacterial cells of the of Escherichia coli strains BLR(DE3)pLysS, BLR(DE3), BLR, BL21(DE3)pLysS, BL21(DE3)pLysE, BL21(DE3), BL21, BL21-SI, BL21 Star, HMS174(DE3)pLysE, HMS174(DE3), HMS174, NovaBlue(DE3), NovaBlue, DH5α, DH5αF′ or DH5αF′IQ

In general, plasmid vectors containing replicon and control sequences, which are derived from species compatible with the host cell are used in connection with these hosts. The vector ordinarily carries a replication site, as well as marking sequences which are capable of providing phenotypic selection in transformed cells. For example, E. coli is transformed using pBR322, a plasmid derived from an E. coli species (Bolivar, et al. 1977). pBR322 contains genes for ampicillin and tetracycline resistance and thus provides an easy means for identifying transformed cells. The pBR plasmid, or other microbial plasmid or phage must also contain, or be modified to contain, promoters which can be used by the microbial organism for expression of its own polypeptides.

Those promoters most commonly used in recombinant DNA construction include the β-lactamase (penicillinase) and lactose promoter systems (Chang, et al. 1978; Itakura., et al. 1977, Goeddel, et al. 1979; Goeddel, et al. 1980) and a tryptophan (TRP) promoter system. Contemplated for use in the present invention is the T7 promoter. While these are the most commonly used, other microbial promoters have been discovered and utilized, and details concerning their nucleotide sequences have been published, enabling a skilled worker to introduce functional promoters into plasmid vectors (Siebwenlist, et al. 1980).

Means of transforming or transfecting cells with exogenous polynucleotide such as DNA molecules are well known in the art and include techniques such as calcium-phosphate- or DEAE-dextran-mediated transfection, protoplast fusion, electroporation (see e.g., Sambrook, Fritsch and Maniatis, 1989).

The most widely used method is transfection mediated by either calcium phosphate or DEAE-dextran. Although the mechanism remains obscure, it is believed that the transfected DNA enters the cytoplasm of the cell by endocytosis and is transported to the nucleus. Depending on the cell type, up to 90% of a population of cultured cells can be transfected at any one time. Because of its high efficiency, transfection mediated by calcium phosphate or DEAE-dextran is the method of choice for experiments that require transient expression of the foreign DNA in large numbers of cells. Calcium phosphate-mediated transfection is also used to establish cell lines that integrate copies of the foreign DNA, which are usually arranged in head-to-tail tandem arrays into the host cell genome.

The application of brief, high-voltage electric pulses to a variety of prokaryotic and plant cells leads to the formation of nanometer-sized pores in the bacterial membrane. DNA is taken directly into the cell cytoplasm either through these pores or as a consequence of the redistribution of membrane components that accompanies closure of the pores. Electroporation can be extremely efficient method for moving DNA through the cell membrane.

A transfected cell can be prokaryotic or eukaryotic. Preferably, the host cells of the invention are prokaryotic host cells. Where it is of interest to produce a Neisseria meningitidis polypeptide, cultured prokaryotic host cells are of particular interest.

In yet another embodiment, the present invention contemplates a process or method of preparing Neisseria meningitidis polypeptides comprising transforming, transfecting or infecting cells with a polynucleotide that encodes a Neisseria meningitidis polypeptide to produce transformed host cells; and maintaining the transformed host cells under biological conditions sufficient for expression of the polypeptide. Preferably, the transformed host cells are prokaryotic cells. More preferably, the prokaryotic cells are bacterial cells of the BLR (DE3) pLysS strain of Escherichia coli. Even more preferably, the polynucleotide transfected into the transformed cells comprise the nucleic acid sequence of SEQ ID NO:1 (wherein codon 18 is TAC), SEQ ID NO:3 (wherein codon 18 is TAC), SEQ ID NO: 5, SEQ ID NO: 7, SEQ ID NO: 9, SEQ ID NO: 11, SEQ ID NO: 13 (wherein codon 18 is TAC), SEQ ID NO: 15 (wherein codon 18 is TAC), SEQ ID NO: 17 SEQ ID NO: 19 or SEQ ID NO:24 (wherein codon 18 is TAC). Additionally, transfection is accomplished using an expression vector disclosed above. A host cell used in the process is capable of expressing a functional (i.e., antigenic), recombinant Neisseria meningitidis polypeptide.

Following transfection, the cell is maintained under culture conditions for a period of time sufficient for expression of a Neisseria meningitidis polypeptide. Culture conditions are well known in the art and include ionic composition and concentration, temperature, pH and the like. Typically, transfected cells are maintained under culture conditions in a culture medium. Suitable media for various cell types are well known in the art. In a preferred embodiment, temperature is from about 20° C. to about 50° C., more preferably from about 30° C. to about 40° C. and, even more preferably about 37° C.

The pH is preferably from about a value of 6.0 to a value of about 8.0, more preferably from about a value of about 6.8 to a value of about 7.8 and, most preferably about 7.4. Osmolality is preferably from about 200 milliosmols per liter (mosm/L) to about 400 mosm/l and, more preferably from about 290 mosm/L to about 310 mosm/L. Other biological conditions needed for transfection and expression of an encoded protein are well known in the art.

Transfected cells are maintained for a period of time sufficient for expression of a Neisseria meningitidis polypeptide. A suitable time depends inter alia upon the cell type used and is readily determinable by a skilled artisan. Typically, maintenance time is from about 1 to 2 days.

Recombinant Neisseria meningitidis polypeptide is recovered or collected either from the transfected cells or the medium in which those cells are cultured. Recovery comprises isolating and purifying the Neisseria meningitidis polypeptide. Isolation and purification techniques for polypeptides are well known in the art and include such procedures as precipitation, filtration, chromatography, electrophoresis and the like.

D. Immunogenic Compositions and Antibodies

The isolated polynucleotides of the invention are used to express Neisseria meningitidis polypeptides (e.g., via a recombinant expression vector in a host cell as described above). Moreover, anti-Neisseria meningitidis antibodies are used to detect and isolate a Neisseria meningitidis porin polypeptide (or a fragment thereof present in a biological sample.

In particular embodiments, the invention provides immunogenic Neisseria meningitidis antigen compositions comprising polypeptides having an amino acid sequence of SEQ ID NO:2 (wherein the amino acid at residue 18 is a Tyr) and/or SEQ ID NO:4 (wherein the amino acid at residue 18 is a Tyr) and/or SEQ ID NO:14 (wherein the amino acid at residue 18 is a Tyr) and/or SEQ ID NO:16 (wherein the amino acid at residue 18 is a Tyr) and/or SEQ ID NO:25 (wherein the amino acid at residue 18 is a Tyr). In other embodiments, an immunogenic composition further comprises additional Neisseria meningitidis antigens than those set forth in SEQ ID Nos:2, 4, 14, 16 and 25, such as newly identified mature or endogenous Neisseria meningitidis sequences optimized for increased expression in a host cell. The immunogenic composition may further comprise a pharmaceutically acceptable carrier, as outlined in Section E. In certain preferred embodiments, the immunogenic composition will comprise one or more adjuvants. As defined hereinafter, an “adjuvant” is a substance that serves to enhance the immune response to an “antigen”. Thus, adjuvants are often given to boost the immune response and are well known to the skilled artisan.

Examples of adjuvants contemplated in the present invention include, but are not limited to, aluminum salts (alum) such as aluminum phosphate and aluminum hydroxide, Mycobacterium tuberculosis, Bordetella pertussis, bacterial lipopolysaccharides, aminoalkyl glucosamine phosphate compounds (AGP), or derivatives or analogs thereof, which are available from Corixa (Hamilton, Mont.), and which are described in U.S. Pat. No. 6,113,918; one such AGP is 2-[(R)-3-Tetradecanoyloxytetradecanoylamino]ethyl 2-Deoxy-4-O-phosphono-3-O-[(R)-3-tetradecanoyoxytetradecanoyl]-2-[(R)-3-tetradecanoyoxytetradecanoylamino]-b-D-glucopyranoside, which is also known as 529 (formerly known as RC529), which is formulated as an aqueous form or as a stable emulsion, MPL™ (3-O-deacylated monophosphoryl lipid A) (Corixa) described in U.S. Pat. No. 4,912,094, synthetic polynucleotides such as oligonucleotides containing a CpG motif (U.S. Pat. No. 6,207,646), polypeptides, saponins such as Quil A or STIMULON™ QS-21 (Antigenics, Framingham, Mass.), described in U.S. Pat. No. 5,057,540, a pertussis toxin (PT), or an E. coli heat-labile toxin (LT), particularly LT-K63, LT-R72, CT-S109, PT-K9/G129; see, e.g., International Patent Publication Nos. WO 93/13302 and WO 92/19265, cholera toxin (either in a wild-type or mutant form, e.g., wherein the glutamic acid at amino acid position 29 is replaced by another amino acid, preferably a histidine, in accordance with published International Patent Application number WO 00/18434).

Various cytokines and lymphokines are suitable for use as adjuvants. One such adjuvant is granulocyte-macrophage colony stimulating factor (GM-CSF), which has a nucleotide sequence as described in U.S. Pat. No. 5,078,996. A plasmid containing GM-CSF cDNA has been transformed into E. coli and has been deposited with the American Type Culture Collection (ATCC), 1081 University Boulevard, Manassas, Va. 20110-2209, under Accession Number 39900. The cytokine lnterleukin-12 (IL-12) is another adjuvant which is described in U.S. Pat. No. 5,723,127. Other cytokines or lymphokines have been shown to have immune modulating activity, including, but not limited to, the interleukins 1-α, 1-β, 2, 4, 5, 6, 7, 8, 10, 13, 14, 15, 16, 17 and 18, the interferons-α, β and γ, granulocyte colony stimulating factor, and the tumor necrosis factors α and β, and are suitable for use as adjuvants.

Provided also in the invention are methods for immunizing a host against Neisseria meningitidis infection. In a preferred embodiment, the host is human. Thus, a host (or subject) is administered an immunizing amount of an immunogenic composition comprising at least a PorA polypeptide having an amino acid sequence of SEQ ID NO:2 (wherein the amino acid at residue 18 is a Tyr) and/or SEQ ID NO:4 (wherein the amino acid at residue 18 is a Tyr) and/or SEQ ID NO:14 (wherein the amino acid at residue 18 is a Tyr) and/or SEQ ID NO:16 (wherein the amino acid at residue 18 is a Tyr), a biological equivalent thereof or a fragment thereof and a pharmaceutically acceptable carrier. In certain preferred embodiments, a multivalent immunogenic composition (e.g., a six valent composition, a seven valent composition, an eight valent composition, a nine valent composition, etc.) comprises one or more PorA polypeptides having an amino acid sequence of SEQ ID NO:2 (wherein the amino acid at residue 18 is a Tyr), SEQ ID NO:4 (wherein the amino acid at residue 18 is a Tyr), SEQ ID NO: 6, SEQ ID NO: 8, SEQ ID NO: 10, SEQ ID NO: 12, SEQ ID NO: 14 (wherein the amino acid at residue 18 is a Tyr), SEQ ID NO:16 (wherein the amino acid at residue 18 is a Tyr), SEQ ID NO: 18, SEQ ID NO: 20 and SEQ ID NO:25 (wherein the amino acid at residue 18 is a Tyr). An immunizing amount of an immunogenic composition is determined by doing a dose response study in which subjects are immunized with gradually increasing amounts of the immunogenic composition and the immune response analyzed to determine the optimal dosage. Starting points for the study can be inferred from immunization data in animal models. The dosage amount varies depending upon specific conditions of the individual. The amount can be determined in routine trials by means known to those skilled in the art.

An immunologically effective amount of the immunogenic composition in an appropriate number of doses is administered to the subject to elicit an immune response. Immunologically effective amount, as used herein, means the administration of that amount to a mammalian host (preferably human), either in a single dose or as part of a series of doses, sufficient to at least cause the immune system of the individual treated to generate a response that reduces the clinical impact of the bacterial infection. Protection may be conferred by a single dose of the immunogenic composition or vaccine, or may require the administration of several doses, in addition to booster doses at later times to maintain protection. This may range from a minimal decrease in bacterial burden to prevention of the infection. Ideally, the treated individual will not exhibit the more serious clinical manifestations of the Neisseria meningitidis infection. The dosage amount can vary depending upon specific conditions of the individual, such as age and weight. This amount can be determined in routine trials by means known to those skilled in the art.

The peptides and proteins of the invention are formulated as univalent and multivalent immunogenic compositions. In a certain embodiments, an immunogenic composition of the invention is a six valent, a seven valent, an eight valent or a nine valent immunogenic composition. In other embodiments, the peptides and proteins of the invention (e.g., SEQ ID Nos:2, 4, 14, 16 and 25) are administered as multivalent immunogenic compositions in combination with other antigens of Neisseria meningitidis. For example, the peptides and proteins are administered in conjunction with additional Neisseria meningitidis outer membrane proteins or antigenic polysaccharide. In one particular embodiment, the peptides and proteins of the invention are administered in combination with a Neisseria protein encoded by a nucleic acid sequence open reading frame (ORF) identified as “ORF2086”.

The ORF2086 nucleic acid sequence encodes a protein antigen first observed in a complex mixture of soluble outer membrane proteins (OMPs) from a meningococcal strain. The isolated and purified ORF2086 protein antigen exhibited bactericidal activity against at least six of the Neisseria meningitidis serosubtypes, as described in International Publication No. WO 03/063766 A2 (International Application No. PCT/US02/32369) and U.S. Continuation-In-Part Application No. 60//463,161, filed Apr. 16, 2003 (each specifically incorporated herein by reference in its entirety).

In certain embodiments, an ORF2086 protein comprises any of the following amino acid sequences: ADIGxGLADA (SEQ ID NO:26), wherein x is any amino acid; IGxGLADALT (SEQ ID NO:27), wherein x is any amino acid; SLNTGKLKND (SEQ ID NO:28); SLNTGKLKNDKxSRFDF (SEQ ID NO:29), wherein x is any amino acid; SGEFQxYKQ (SEQ ID NO:30), wherein x is any amino acid; IEHLKxPE (SEQ ID NO:31), wherein x is any amino acid; or combinations thereof.

In certain other embodiments, an ORF2086 protein is a Neisseria Subfamily A protein comprising any of the following amino acid sequences: GGGVAADIGx (SEQ ID NO:32), wherein x is any amino acid; SGEFQIYKQ (SEQ ID NO:33); HSAVVALQIE (SEQ ID NO:34); EKINNPDKID (SEQ ID NO:35); SLINQRSFLV (SEQ ID NO:36); SGLGGEHTAF (SEQ ID NO:37); GEHTAFNQLP (SEQ ID NO:38); SFLVSGLGGEH (SEQ ID NO:39); EKINNPDKIDSLINQRSFLVSGLGGEHTAFNQLP (SEQ ID NO:40); GKAEYHGKAF (SEQ ID NO:41); YHGKAFSSDD (SEQ ID NO:42); GKAEYHGKAFSSDD (SEQ ID NO:43); IEHLKTPEQN (SEQ ID NO 44); KTPEQNVELA (SEQ ID NO:45); IEHLKTPEQNVELA (SEQ ID NO:46); AELKADEKSH (SEQ ID NO:47); AVILGDTRYG (SEQ ID NO:48); AELKADEKSHAVILGDTRYG (SEQ ID NO:49); EEKGTYHLAL (SEQ ID NO:50); KINNPDKIDSLINQ (SEQ ID NO:51); DEKSHAVILG (SEQ ID NO:52); KIGEKVHEIG (SEQ ID NO:53) and combinations thereof.

In certain other embodiments, an ORF2086 protein is a Neisseria Subfamily B protein comprising any of the following amino acid sequences: LITLESGEFQ (SEQ ID NO:54); SALTALQTEQ (SEQ ID NO:55); FQVYKQSHSA (SEQ ID NO:56); LITLESGEFQVYKQSHSALTALQTEQ (SEQ ID NO:57); IGDIAGEHTS (SEQ ID NO:58); EHTSFDKLPK (SEQ ID NO:59); IGDIAGEHTSFDKLPK (SEQ ID NO:60); ATYRGTAFGS (SEQ ID NO:61); DDAGGKLTYT (SEQ ID NO:62); IDFAAKQGHG (SEQ ID NO:63); KIEHLKSPEL (SEQ ID NO:64); ATYRGTAFGSDDAGGKLTYTIDFAAKQGHGKIEHLKSPELNV (SEQ ID NO:65); HAVISGSVLY (SEQ ID NO:66); KGSYSLGIFG (SEQ ID NO:67); VLYNQDEKGS (SEQ ID NO:68); HAVISGSVLYNQDEKGSYSLGIFG (SEQ ID NO:69); AQEVAGSAEV (SEQ ID NO:70); IHHIGLAAKQ (SEQ ID NO:71); VETANGIHHI (SEQ ID NO:72); AQEVAGSAEVETANGIHHIGLAAKQ (SEQ ID NO:73); VAGSAEVETANGIHHIGLAAKQ (SEQ ID NO:74); MVAKRQFRIG (SEQ ID NO:75); DIAGEHTSFDKLP (SEQ ID NO:76); YTIDFAAKQG (SEQ ID NO:77); GKIEHLKSPELNV (SEQ ID NO:78); HAVISGSVLYNQ (SEQ ID NO:79); AQEVAGSAEV (SEQ ID NO:80) and combinations thereof.

In another embodiment, an ORF2086 protein comprises a consensus sequence of SEQ ID NO:81 and/or immunogenic portions thereof.

ORF2086 Protein Consensus Sequence (SEQ ID NO:81):
CSSG-----GGGVxADIGxGLADALTxPxDxKDKxLxSLTLxxSxxxNxx
LxLxAQGAEKTxxxGD---SLNTGKLKNDKxSRFDFxxxIxVDGxxITLx
SGEFQxYKQxHSAxxALQxExxxxxxxxxxxxxxRxFxxxxxxGEHTxFx
xLPxx-xAxYxGxAFxSDDxxGxLxYxIDFxxKQGxGxIEHLKxPExNVx
LAxxxxKxDEKxHAVIxGxxxYxxxEKGxYxLxxxGxxAQExAGxAxVxx
xxxxHxIxxAxKQ

In the foregoing consensus sequence, the “x” represents any amino acid, the region from amino acid position 5 to amino acid position 9 is any of 0 to 5 amino acids, the region from amino acid position 67 to amino acid position 69 is any of 0 to 3 amino acids, and amino acid position 156 is any of 0 to 1 amino acid. In one particular embodiment, the region from amino acid position 5 to amino acid position 9 comprises 0, 4 or 5 amino acids and the region from amino acid position 67 to amino acid position 69 comprises 0 or 3 amino acids.

In certain other embodiments, an ORF2086 protein of Subfamily A comprises a consensus sequence of SEQ ID NO:82 and/or immunogenic portions thereof.

2086 Subfamily A sequence (SEQ ID NO:82):
CSSG----GGGVAADIGxGLADALTxPxDxKDKxLxSLTLxxSxxxNxxL
xLxAQGAEKTxxxGD---SLNTGKLKNDKxSRFDFxxxIxVDGQxITLxS
GEFQIYKQxHSAVVALQIEKINNPDKIDSLINQRSFLVSGLGGEHTAFNQ
LPxGKAEYHGKAFSSDDxxGxLxYxIDFxxKQGxGxIEHLKTPEQNVELA
xAELKADEKSHAVILGDTRYGxEEKGTYHLALxGDRAQEIAGxATVKIxE
KVHEIxIAxKQ

The reference “x” is any amino acid. The region from amino acid position 5 to amino acid position 8 is any of 0 to 4 amino acids. The region from amino acid position 66 to amino acid position 68 is any of 0 to 3 amino acids. In one particular embodiment, the region from amino acid position 5 to amino acid position 8 comprises 0 or 4 amino acids and the region from amino acid position 66 to amino acid position 68 comprises 0 or 3 amino acids.

In certain other embodiments, an ORF2086 protein of Subfamily B comprises a consensus sequence of SEQ ID NO:83 and/or immunogenic portions thereof.

2086 Subfamily B (SEQ ID 83):
CSSGGGG-----VxADIGxGLADALTAPLDHKDKxLxSLTLxxSxxxNxx
LxLxAQGAEKTYGNGDSLNTGKLKNDKVSRFDFIRQIEVDGxLITLESGE
FQVYKQSHSALTALQTEQxQDxExSxKMVAKRxFxIGDIAGEHTSFDKLP
KxxxATYRGTAFGSDDAGGKLTYTIDFAAKQGHGKIEHLKSPELNVxLAx
xYIKPDEKxHAVISGSVLYNQDEKGSYSLGIFGxxAQEVAGSAEVETANG
IHHIGLAAKQ

The reference “x” is any amino acid. The region from amino acid position 8 to amino acid position 12 is any of 0 to 5 amino acids. In one particular embodiment, the region from amino acid position 8 to amino acid position 12 comprises 0 or 5 amino acids.

The immunogenic compositions are administered to a human or animal in a variety of ways. These include intradermal, intramuscular, intraperitoneal, intravenous, subcutaneous, oral and intranasal routes of administration.

In another embodiment, the present invention provides antibodies immunoreactive with porin polypeptides. Preferably, the antibodies of the invention are monoclonal antibodies. Additionally, the porin polypeptides are PorA polypeptides which comprise the amino acid residue sequence of SEQ ID NO:2 (wherein the amino acid at residue 18 is a Tyr) and/or SEQ ID NO:4 (wherein the amino acid at residue 18 is a Tyr) and/or SEQ ID NO:14 (wherein the amino acid at residue 18 is a Tyr) and/or SEQ ID NO:16 (wherein the amino acid at residue 18 is a Tyr) and/or SEQ ID NO:25 (wherein the amino acid at residue 18 is a Tyr). Means for preparing and characterizing antibodies are well known in the art (see, e.g., Antibodies “A Laboratory Manual, E. Howell and D. Lane, Cold Spring Harbor Laboratory, 1988).

As used herein, an antibody is said to selectively bind to a polypeptide of the invention when the antibody binds to the desired polypeptide and does not selectively bind to unrelated proteins.

The term “antibody” as used herein refers to immunoglobulin molecules and immunologically active fragments of immunoglobulin molecules, i.e., molecules that contain an antigen binding site which specifically binds (immunoreacts with) an antigen, such as SEQ ID NO:2 (wherein the amino acid at residue 18 is a Tyr), SEQ ID NO:4 (wherein the amino acid at residue 18 is a Tyr), SEQ ID NO:14 (wherein the amino acid at residue 18 is a Tyr), SEQ ID NO:16 (wherein the amino acid at residue 18 is a Tyr) or SEQ ID NO:25 (wherein the amino acid at residue 18 is a Tyr). The invention provides polyclonal and monoclonal antibodies that bind porin proteins. The term “monoclonal antibody” or “monoclonal antibody composition,” as used herein, refers to a population of antibody molecules that contain only one species of an antigen binding site capable of immunoreacting with a particular epitope of porin (e.g. a PorA epitope). A monoclonal antibody composition thus typically displays a single binding affinity for a particular polypeptide with which it immunoreacts.

To generate anti-porin antibodies, an isolated porin polypeptide, or a fragment thereof, is used as an immunogen to generate antibodies that bind porin using standard techniques for polyclonal and monoclonal antibody preparation. A full-length porin polypeptide can be used or, alternatively, an antigenic peptide fragment of porin can be used as an immunogen. An antigenic fragment of the porin polypeptide will typically comprises at least 8 contiguous amino acid residues, e.g., 8 contiguous amino acids from SEQ ID NO:2, SEQ ID NO:4, SEQ ID NO:14, SEQ ID NO:16 or SEQ ID NO:25. Preferably, the antigenic peptide comprises at least 10 amino acid residues, more preferably at least 15 amino acid residues, even more preferably at least 20 amino acid residues, and most preferably at least 30 amino acid residues of a porin polypeptide. Preferred fragments for generating anti-porin antibodies are regions of a porin polypeptide that are located on the surface of the polypeptide, e.g., hydrophilic regions, and more desirable on the outer surface of Neisseria.

A monoclonal antibody of the present invention is readily prepared through use of well-known techniques such as those exemplified in U.S. Pat. No. 4,196,265, herein incorporated by reference.

By use of a monoclonal antibody of the present invention, specific polypeptides and polynucleotide of the invention can be recognized as antigens, and thus identified. Once identified, those polypeptides and polynucleotide can be isolated and purified by techniques such as antibody-affinity chromatography. In antibody-affinity chromatography, a monoclonal antibody is bound to a solid substrate and exposed to a solution containing the desired antigen. The antigen is removed from the solution through an immunospecific reaction with the bound antibody. The polypeptide or polynucleotide is then easily removed from the substrate and purified.

Additionally, examples of methods and reagents particularly amenable for use in generating and screening antibody display library can be found in, for example, U.S. Pat. No. 5,223,409; International Application No. WO 92/18619; International Application No. WO 91/17271; International Application No. WO 92/20791; International Application No. WO 92/15679; International Application No. WO 93/01288; International Application No. WO 92/01047; International Application No. WO 92/09690 and International Application No. WO 90/02809.

Additionally, antibodies, such as chimeric and humanized monoclonal antibodies, comprising both human and non-human fragments, are made using standard recombinant DNA techniques, for example using methods described in European Application Nos. EP 184,187; EP 171,496; EP 173,494; International Application No. WO 86/01533; U.S. Pat. No. 4,816,567; and European Application No. EP 125,023.

E. Pharmaceutical Compositions

In certain embodiments, the present invention provides pharmaceutical and immunogenic compositions comprising Neisseria meningitidis polypeptides and physiologically acceptable carriers. More preferably, the pharmaceutical compositions comprise Neisseria meningitidis PorA polypeptides comprising the amino acid residue sequence of SEQ ID NO:2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20 or SEQ ID NO:25.

The Neisseria meningitidis PorA proteins or polypeptides (also referred to hereinafter as “active compounds”) of the invention are incorporated into pharmaceutical compositions suitable for administration to a subject, e.g., a human. Such compositions typically comprise the nucleic acid molecule, protein, modulator, or antibody and a pharmaceutically acceptable carrier. As used hereinafter the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions.

A pharmaceutical composition of the invention is formulated to be compatible with its intended route of administration. Examples of routes of administration include parenteral (e.g., intravenous, intradermal, subcutaneous, intramuscular, intraperitoneal), mucosal (e.g., oral, rectal, intranasal, buccal, vaginal, respiratory) and transdermal (topical). Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier is a solvent or dispersion medium containing, for example, water, ethanol, polyol (for example, glycerol, propylene glycol, and liquid polyetheylene glycol, and the like), and suitable mixtures thereof. The proper fluidity is maintained, for example, by the use of a coating such as lecithin, by the maintenance of the required particle size in the case of dispersion and by the use of surfactants. Prevention of the action of microorganisms is achieved by various antibacterial and antifungal agents, for example, parabens, chlorobutanol, phenol, ascorbic acid, thimerosal, and the like. In many cases, it is preferable to include isotonic agents, for example, sugars, polyalcohols such as manitol, sorbitol, sodium chloride in the composition. Prolonged absorption of the injectable compositions are brought about by including in the composition an agent which delays absorption, for example, aluminum monostearate and gelatin.

Sterile injectable solutions can be prepared by incorporating the active compound (e.g., a Neisseria meningitidis PorA polypeptide) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They are enclosed in gelatin capsules or compressed into tablets. For the purpose of oral therapeutic administration, the active compound is incorporated with excipients and used in the form of tablets, troches, or capsules. Oral compositions also are prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer. Systemic administration can also be by transmucosal or transdermal means. For mucosal or transdermal administration, penetrants appropriate to the barrier to be permeated are used in the formulation. Such penetrants are generally known in the art, and include, for example, for mucosal administration, detergents, bile salts, and fusidic acid derivatives. Mucosal administration is accomplished through the use of nasal sprays or suppositories. For transdermal administration, the active compounds are formulated into ointments, salves, gels, or creams as generally known in the art.

The compounds also are be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems.

Biodegradable, biocompatible polymers are used, such as ethylene vinyl acetate, polyanhydrides, polyglycolic acid, collagen, polyorthoesters, and polylactic acid. Methods for preparation of such formulations are apparent to those skilled in the art. The materials can also be obtained commercially from Alza corporation and Nova Pharmaceuticals, Inc. Liposomal suspensions (including liposomes targeted to infected cells with monoclonal antibodies to viral antigens) can also be used as pharmaceutically acceptable carriers. These are prepared according to methods known to those skilled in the art, for example, as described in U.S. Pat. No. 4,522,811, incorporated herein by reference in its entirety.

It is especially advantageous to formulate oral or parenteral compositions in dosage unit form for ease of administration and uniformity of dosage. Dosage unit form as used hereinafter refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

A pharmaceutically acceptable vehicle is understood to designate a compound or a combination of compounds entering into a pharmaceutical or immunogenic composition which does not cause side effects and which makes it possible, for example, to facilitate the administration of the active compound, to increase its life and/or its efficacy in the body, to increase its solubility in solution or alternatively to enhance its preservation. These pharmaceutically acceptable vehicles are well known and will be adapted by persons skilled in the art according to the nature and the mode of administration of the active compound chosen.

A composition of the present invention is typically administered parenterally in dosage unit formulations containing standard, well-known nontoxic physiologically acceptable carriers, adjuvants, and vehicles as desired. The term parenteral as used hereinafter includes intravenous, intramuscular, intraarterial injection, or infusion techniques.

Injectable preparations, for example sterile injectable aqueous or oleaginous suspensions, are formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol.

Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid find use in the preparation of injectables.

A carrier is also a liposome. Means for using liposomes as delivery vehicles are well known in the art (see, e.g. Gabizon et al., 1990; Ferruti et at., 1986; and Ranade, V. V., 1989).

An assay is used to confirm that the polynucleotides administered by immunization do not give rise to a transformed phenotype in the host (U.S. Pat. No. 6,168,918, incorporated herein by reference in its entirety).

All patents and publications cited herein are incorporated by reference.

### F. EXAMPLES

The following examples are carried out using standard techniques, which are well known and routine to those of skill in the art, except where otherwise described in detail. The following examples are presented for illustrative purpose, and should not be construed in any way limiting the scope of this invention.

### Example 1

### Materials and Methods

The endogenous porA genes were obtained from seven clinical isolates of serogroup B Neisseria meningitidis. The strains are listed by a designated name with their serogroup, serotypes and serosubtypes shown in parentheses; H355 (B:15, P1:19,15), 6557 (B:7, P1:22a,14), NMB (B:2b, P1:5a,2c), 870227 (B:4, P1:5c,10), H44/76 (B:15, P1:7,16), 880049 (B:4, P1:7b,4), M97 23462 (B:4z, P1:22,14).

Each porA gene was amplified by polymerase chain reaction (PCR) (AmpliTaq and ABI 2400 thermal cycler, Applied Biosystems, Foster City, Calif.) from chromosomal DNA derived from the above listed strains. The PCR amplification of the porA genes utilized two oligonucleotide primers (Table 4) in each reaction: PORABGL2 (SEQ ID NO:21) and NMBGL2TR (SEQ ID NO:22) or PI10M18 (SEQ ID NO:23) and NMBGL2TR (SEQ ID NO:22). The amplified porA PCR products were cloned directly into the TOPO-PCR2.1 cloning vector and selected on HySoy agar supplemented with 100 μg/ml ampicillin and 20 pg/ml X-Gal. White colonies were selected and grown. Plasmid DNA was prepared using a Qiagen miniprep kit and the plasmids were screened for the PCR fragment insert. PCR insert plasmids were subjected to DNA sequencing (Big Dye chemistry on an ABI377 sequencer, Applied Biosystems, Foster City, Calif.).

TABLE 4
PCR AMPLIFICATION PRIMERS

PORABGL2
5′-CGCGAGATCTCATATGGATGTCAGCCTATACGGCGAAATCAAAGC-
3′
(SEQ ID NO:21)
NMBGL2TR
3′-CGTCGGTTTGCGCCACAAATTCTAATGAGTGACTGAAGATCTCGCG-
5′
(SEQ ID NO:22)
PI10AA18
5′-CGCGAGATCTCATATGGATGTCAGCCTATACGGCGAAATCAAAGCCG
GCGTGGAAGGCAGGAACTACCAG-3′
(SEQ ID NO:23)

Note:
The start codon (ATG) is underlined in PORABGL2, the multiple stop codons are underlined in NMBGL2TR, both the start codon (ATG) and codon conversion from ATC to TAC are underlined in PI10AA18.

Cloning and Expression in the pET9a Vector

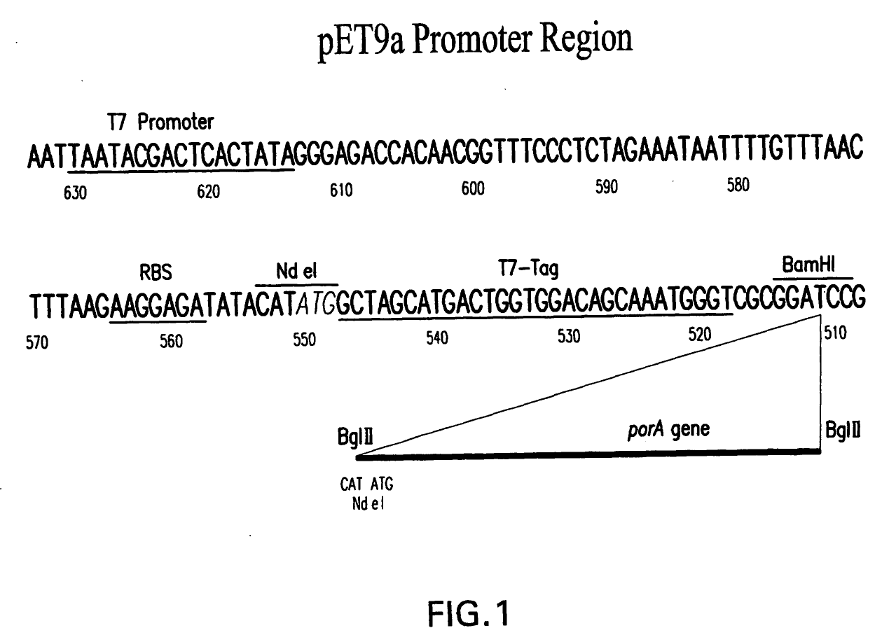

Plasmids exhibiting the correct DNA sequence were digested with BgIII restriction enzyme and the BgIII fragment was gel purified using a GeneClean II purification kit (Bio101, Carlsbad, Calif.). The purified BgIII fragment was cloned into the BamHI site of the expression vector pET9a (FIG. 1). The pET9a/porA host strains were selected on HySoy plates supplemented with 30 μg/ml kanamycin. Kanamycin resistant clones were grown and miniprep plasmid DNA was prepared. The plasmids were screened for the appropriate orientation of the porA gene in the BamHI site. Correctly oriented plasmids represent a fusion of the T7-antigen to the amino terminus of porA gene. These T7-antigen/PorA fusions were transformed into BLR(DE3)pLysS and selected on HySoy/Kan plates. The cultures were grown overnight at 37° C. in HySoy broth supplemented with 1% glucose. The overnight cultures were diluted 1/100 in fresh HySoy/1% glucose broth and grown for 2 hours at 37° C. After 2 hours of growth the cells were at an approximate optical density of 1.0. The cultures were induced to express the T7-Tag/PorA fusion protein by the addition of 1 mM IPTG (isopropyl β-D-thiogalactopyranoside). The induced cells were grown for approximately 2 hours at 37° C. and the cultures were then harvested. Whole cell lysates of approximately 1×10⁸ cells were prepared by the Laemmli protocol. The expression level of the PorA protein was assessed by observation of total meningococcal cellular lysates by polyacrylamide gel electrophoresis (PAGE) and Coommassie Blue staining. The percentage of PorA protein to the total amount of cellular protein was calculated on a Molecular Dynamics densitometer.

Deletion of the T7-Antigen

Each fusion plasmid was then subjected to a Ndel restriction digest, which deletes the T7-antigen and links the mature porA gene directly to the ATG start (i.e., mature +1) provided by the pET vector (FIG. 1). These Ndel deleted plasmids were transformed into Top10 cells and selected on HySoy/Kan plates. Candidate clones were grown and miniprep plasmid DNA was prepared. The plasmid DNA was subjected to DNA sequencing to confirm the deletion and the integrity of the porA gene sequence. Plasmids representing the correct DNA sequence were transformed into BLR(DE3)pLysS, selected on HySoy/Kan plates, grown in HySoy/glucose broth and induced to express PorA with IPTG. The total amount of PorA produced was assessed by densitometry.

Isolation and Solubilization of Recombinant PorA Inclusion Bodies

E. coli frozen cell paste (50 g wet weight) was thawed and resuspended in 250 mL of TE/pH 8.0 buffer and the cells lysed by passage through a microfluidizer. The suspension was centrifuged at 10,000 rpm and the pellet, containing PorA inclusion bodies (IBs), was resuspended in 250 mL TE/pH 8.0 buffer containing 1.0% TX-100. The suspension was stirred at room temperature for 1-2 hours and then centrifuged at 10,000 rpm. The pellet was collected and washed an additional 2 times with TE/pH 8.0/1.0% TX-100. Following the third TX-100 wash, the pellet was resuspended in 250 mL TE/pH 8.0 buffer containing 1.0% Z3-14, stirred for 1-2 hours, and centrifuged at 10,000 rpm. The pellet was collected and washed a second time with TE/pH 8.0/1.0% Z3-14.

The IB pellet was subsequently denatured and solubilized in 250 mL of TE/pH 8.0 buffer containing 8.0 M urea. Following denaturation, the material was centrifuged at 10,000 rpm and the clarified supernatant collected. TE/pH 8.0 buffer containing 10.0% Z3-14 and 5.0M NaCl was added to the clarified supernatant to give a final concentration of 1.0% Z3-14 and 250 mM NaCl. The PorA protein was then refolded into a soluble conformation by overnight dialysis against 20 L (2 changes) of TE/pH 8.0 buffer containing 0.05% Z3-14 and 250 mM NaCl.

Fractogel SO3-Chromatography of Recombinant PorA's

Following refolding, the preparation was centrifuged at 10,000 rpm and the clarified supernatant concentrated to approximately 80 mL using a Millipore™ ultrafiltration system with a 10,000 MW cutoff membrane. The concentrated preparation was buffer exchanged into 20 mM NaPO₄/0.1% Z3-14/50 mM NaCl/5 mM EDTA pH 6.0 by passage over a 600 mL Sephadex G-25 column. Following buffer exchange, the PorA was applied to a 200 mL Fractogel SO3-column equilibrated in 20mM NaPO₄/0.1% Z3-14/50 mM NaCl/5 mM EDTA/pH 6.0. The column was washed with five column volumes of 20 mM NaPO₄/0.1% Z3-14/5OmM NaCl (pH 6.0) followed by additional 5 column volumes of the same buffer containing 0.05% Z3-14. The bound PorA was eluted with 20 mM NaPO₄/0.05% Z3-14/pH 6.0 containing 1.0 M NaCl. Fractions containing PorA were pooled and buffer exchanged into 10 mM Tris HCl/0.05% Z3-14/150 mM NaCl/pH 7.5 by passage over a 600 mL Sephadex G-25 column. The preparation was diluted to 5 mg/mL with 10 mM Tris-HCl/150 mM NaCl/0.05% Z3-14 (pH 7.5).

Native PorA Purification

Frozen pellets of Neisseria meningitidis deficient in PorB and capsule were resuspended in 10 mM HEPES-NaOH/1 mM EDTA pH 7.4 at 5 ml/g wet cell weight and lysed by Microfluidizer (Microfluidics Corporation Model 110Y). The lysed cell suspension was adjusted to 0.5 M NaCl and centrifuged at 150,000×g for one hour. The total membrane pellet was solubilized in 10 mM HEPES-NaOH/1 mM MgCl₂/1%Triton-X-100 pH 7.4 for one hour and centrifuged at 150,000×g for 1hr. The outer membrane pellet was solubilized in 50 mM Tris-HCl/5mM EDTA/1% Zwittergent 3-14 (buffer A) for one hour and centrifuged at 150,000×g for one hour. The resulting pellet was solubilized in buffer A/0.5 M NaCl for one hour and centrifuged at 150,000×g for one hour. The supernatant was dialyzed against buffer A, a precipitate was removed by centrifugation, and the supernatant was pooled with the first Zwittergent 3-14 supernatant. The dialyzed Zwittergent 3-14 pool was passed over an anion exchange chromatography column and eluted with a 0-1 M NaCl gradient. Fractions containing PorA were pooled and further purified by size exclusion chromatography (buffer A with 150 mM NaCl). Fractions containing PorA were pooled and analyzed by SDS-PAGE (Coomassie stain). All preparations were 85-90% homogeneous by laser densitometry.

### Example 2

### Results and Discussion

The majority of the porA genes express large quantities of protein after IPTG induction in the pET9a system, with or without the T7-Tag fused to the amino terminus, as demonstrated with pPX7300-T7 or pPX7300 respectively. P1:7,16 expressed in pPX7300/BLR(DE3)pLysS is a representative example of a highly induced PorA protein regardless of fusion status.

Most of the serosubtype recombinant strains containing the pET/porA expression vector, could be induced to express the PorA protein at high levels when the T7-Tag fusion sequence was removed from the plasmids. However the PorA serosubtype recombinant strains containing P1:5c,10, P1:5a,2c, P1:22,9, P1:21,16 and P1:22,14, failed to express PorA protein at substantial levels when the T7-Tag fusion sequence was removed from these plasmid vectors. Comparative analysis of all the expressing and non-expressing strains revealed a codon variation at amino acid position eighteen (i.e., mature +1) that correlated with the porA expression phenotype (Table 5). Non-expressing strains required the conversion of codon 18 from an ATC (IIe) to a TAC (Tyr), encoded by primer PI10AA18, to allow for maximum PorA expression.

TABLE 5
Comparative Analysis of Expressing and Non-Expressing Strains

| | | | | |
|---|---|---|---|---|
| Strain | Serosubtype | Vector | AA#18 | Codon |

Expressing Strains

| | | | | |
|---|---|---|---|---|
| H44/76 | P1:7,16 | pPX7300 | Tyr | TAC* |
| 880049 | P1:7b,4 | pPX7301 | Tyr | TAC* |
| H355 | P1:19,15 | pPX7302 | Phe | TTC* |
| 6557 | P1:22a,14 | pPX7304 | Tyr | TAC* |
| 6940 | P1:18,25,6 | pPX7308 | Ile | ATT* |
| M97 252097 | P1:7b,16 | pPX7310 | Tyr | TAC* |

Non-Expressing Strains

| | | | | |
|---|---|---|---|---|
| NMB | P1:5a,2c | pPX7303 | Ile | ATC |
| 870227 | P1:5c,10 | pPX7309 | Ile | ATC |
| 891 | P1:21,16 | pPX7307 | Ile | ATC |
| M982 | P1:22,9 | pPX7321 | Ile | ATC |
| M97 253462 | P1:22,14 | not assigned | Ile | ATC |

Expressing mutants

| NMB | P1:5a,2c | pPX7316 | Tyr | TAC |
|---|---|---|---|---|
| 870227 | P1:5c,10 | pPX7311 | Tyr | TAC |
| 891 | P1:21,16 | pPX7317 | Tyr | TAC |
| M982 | P1:22,9 | pPX7318 | Tyr | TAC |
| *Underlined bases are conserved compared to the non-expressing ATC codon. | | | | |
| Expressing and non-expressing phenotypes refer to recombinant expression of the PorA protein from the T7 promoter encoded on the pET9a vector without a T7-Tag. The first column shows the various meningococcal porA donor strain designations. The second column shows the serosubtype designation of the PorA protein. The third column indicates the plasmid number designation representing that porA gene cloned in the pET9a vector. | | | | |
| # The fourth column shows the amino acid encoded at position 18 of the PorA polypeptide. The fifth column indicates the nucleotide sequence of the codon at position 18. If the nucleotide sequence at codon 18 is ATC, then the vector fails to highly express PorA if the T7-Tag is not fused to the N-terminus. The other nucleotide sequences represented for codon 18 allow full expression of PorA with or without the T7-Tag fused to the N-terminus (including ATT-Ile, i.e. pPX7308). | | | | |

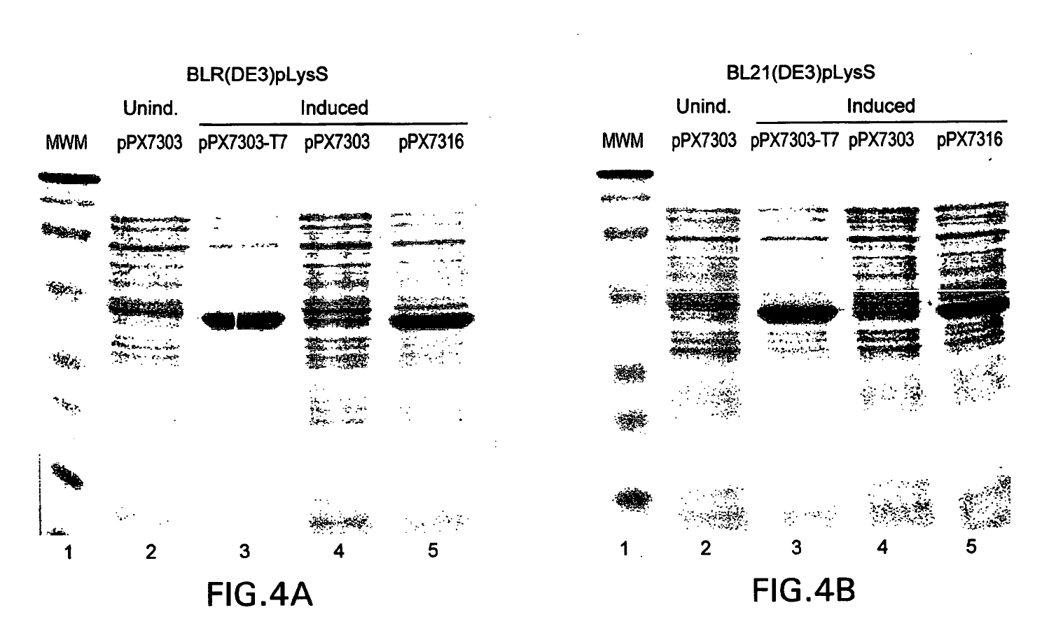

The codon 18 mutations were assigned new plasmid designations: the mutated version of pPX7303 is pPX7316, mutated pPX7309 is pPX7311, mutated pPX7307 is pPX7317 and mutated pPX7321 is pPX7318. The conversion of codon 18 from an ATC to a TAC in pPX7311, pPX7316, pPX7317 and pPX7318 resulted in greatly enhanced expression of the respective PorA proteins. A comparison of induced expression with the tagless P1:5a,2c wild-type PorA gene, T7-Tag/porA fusion gene, and the codon 18 mutant porA gene (pPX7303-T7, pPX7303 and pPX7316 respectively) is shown in FIG. 4. Expression was tested in the E. coli B strains, BLR(DE3)pLysS and BL21 (DE3)pLysS.

Both E. coli B strains demonstrate consistent levels of PorA expression with the different plasmid variants, pPX7303, pPX7303-T7 and pPX7316. Two E. coli K-12 (DE3) derivatives, HMS174(DE3)pLysE and NovaBlue(DE3), were also tested with the same plasmids. A comparison of induced expression with the mature P1:5a,2c wild-type porA gene (pPX7303), 17-Tag/porA fusion gene (pPX7303-T7) and codon 18 mutant (i.e., mature +1) porA gene (pPX7316), in the two K-12 (DE3) derivatives indicated that both pPX7303 and pPX7303-T7 failed to express well in either K-12 strain. Only the mature +1 form of porA with the codon 18 conversion from ATC to TAC expressed well in the K-12 strains, as evidenced by the pPX7316 sample in HMS174 and NovaBlue.

Thus, the pET vector system can express the meningococcal PorA protein as either an amino terminal T7-Tag fusion or with only a methionine fused to the amino terminus of the mature PorA (i.e., mature +1). The only serious problem encountered was the initial failure of four PorA serosubtypes to express the recombinant mature +1 PorA protein at high levels. Site directed mutagenesis of codon 18 of the porA gene restored full expression to these serosubtypes in all of the (DE3) E. coli host strains tested. The protein can be expressed at 30% to 50% of total cellular protein, with or without the T7-Tag fusion. The protein is sequestered in inclusion bodies in the cytoplasm of the cell from which it is purified and refolded. All of the (DE3) lysogenic E. coli strains tested worked well to express PorA.

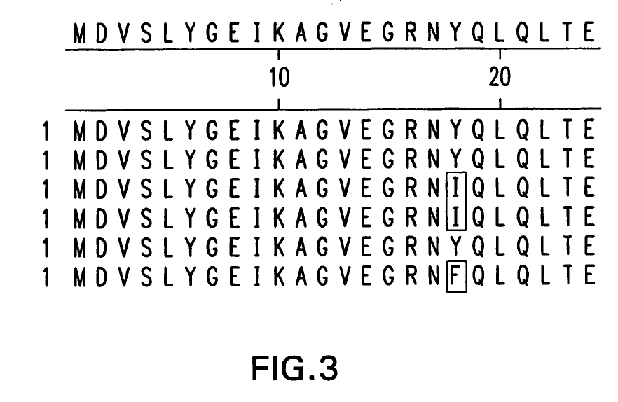

The initial failure of plasmids containing the P1:5c,10, P1:5a,2c, P1:22,9, and P1:21,16 porA genes to express their respective PorA's was overcome by site directed mutagenesis of the inserted porA gene. A comparative analysis of the expressing and non-expressing genes (FIG. 2) showed a single amino acid variation within the first 20 amino acids of the protein (FIG. 3). As noted in Table 5, codon 18 of the non-expressing strains is ATC (IIe), whereas the majority of expressing strains contain a TAC (Tyr) codon with other expressing strains having a TTC (Phe) codon and an ATT (IIe) codon. Conversion of the ATC codon to TAC conveys the expression phenotype. It is also contemplated that identification of an expressing strain (e.g., strain 6940, Table 5) with an ATT (IIe) codon at position 18 indicates that altering the polypeptide composition of PorA is not responsible for the enhanced levels of protein expression, but changes in the nucleotide composition does affect expression. Previous studies have shown that alterations in the 5′ end of the gene coding sequence can have dramatic effects in the level of recombinant protein produced in E. coli. Specifically, silent mutations introduced at the third nucleotide position of various codons within the first 15 codons of the expressed gene (Johansson et al., 1999). These data most likely indicate that the stability or other secondary structure effects of the porA mRNA varies with these nucleotide changes and in turn affects the level of PorA expression. However the exact mechanism at work here has not been identified.

Thus, fusionless porA genes with the ATC codon at position 18 fail to express in all the (DE3) lysogenic strains tested. The TAC conversion restores expression in all the strains tested, any of which could be used in immunogenic compositions. Finally, even T7-Tag fusion proteins failed to highly express the PorA protein in the E. coli K-12 derivatives (HMS174 and NovaBlue), unless the codon at position 18 was changed to TAC (data not shown). However E. coli B strains (BL21 and BLR) express the ATC version as long as the T7-Tag is present (FIG. 4).

In the case of P1:22, 14, a different donor strain of the same serosubtype was used as the source of the porA gene (e.g. the P1:22, 14 porA gene from strain M97 253462 has an ATC (IIe) codon at position 18 and failed to express without the T7-tag, whereas the porA gene from strain 6557 has a TAC (Tyr) at position 18 and expressed at high levels without the T7-tag).

### Example 3

### Methods for Identifying and Increasing the Expression Levels of Neisseria Meningitidis Polypeptides

A comparative analysis of recombinant expressing strains and recombinant non-expressing strains of Neisseria meningitidis porA DNA sequence (see Table 5, Example 2) revealed a codon variation at amino acid position 18 of the PorA (mature +1) polypeptide. It was demonstrated in Example 2, that a mutation of codon 18 from an ATC to a TAC resulted in an increase of PorA polypeptide expression in the non-expressing strains.

As defined previously in the Detailed Description of the Invention, an “endogenous” Neisseria polynucleotide sequence is a polynucleotide isolated or identified from a naturally occurring Neisseria strain and encodes a 5′ signal (or transport or leader) peptide sequence of approximately 19 amino acids. Similarly, an “endogenous” Neisseria protein or polypeptide sequence is a Neisseria protein or polypeptide isolated or identified from a naturally occurring Neisseria strain and comprises a N-terminal signal (or transport or leader) peptide sequence of approximately 19 amino adds, wherein a signal peptidase recognizes the N-terminal signal sequence via a proline turn at amino acid position −6, an alanine at amino acid position −3 and an alanine at amino acid position −1. As defined, a signal sequence generally exhibits three distinct features: (1) a membrane spanning hydrophobic domain, (2) followed by a turn in the peptide sequence formed by either a proline or glycine at approximately amino acid position −6, relative to the cleavage site and (3) in general either an alanine, glycine or serine at both the −3 and −1 positions, relative to the cleavage site (Pugsley, 1993).

In certain embodiments, when analyzing an “endogenous” Neisseria polynucleotide sequence (e.g., in silico), the 5′ nucleotides encoding the approximately 19 amino acids of N-terminal signal sequence may be “hypothetically” deleted to identify the “mature” polynucleotide sequence. Computer programs such as SignalP, Sigcleave or SPScan can be used to predict the signal sequence of a protein and are well known in the art (Zagursky and Russell, 2001, specifically incorporated by reference herein in its entirety). Thus, following the identification of the N-terminal signal sequence via physical inspection or computer program, a person of skill in the art can hypothetically remove the signal sequence to determine the “mature” Neisseria polynucleotide sequence, wherein codon 17 of the “mature” sequence may be mutated to obtain increased expression levels (as described below) or an alternative Neisseria strain may be selected (as described below).

As defined previously in the Detailed Description of the Invention, a “mature” Neisseria polynucleotide sequence is lacking the 5′ nucleotides encoding the signal sequence found in the “endogenous” Neisseria polynucleotide sequence. A “mature” Neisseria protein or polypeptide sequence of the invention is a protein or polypeptide sequence having its N-terminal signal peptide sequence removed (e.g., enzymatically cleaved or deleted from the 5′ nucleotide sequence).

Thus, in one non-limiting example, a method for identifying “mature” Neisseria polynucleotide sequences encoding porin polypeptides expressed at low levels in a host cell comprises:

Method I:

(a) obtaining a “mature” Neisseria polynucleotide sequence; and
(b) determining the triplet sequence at codon 17, wherein an ATC at codon 17 indicates that the encoded porin protein or polypeptide is expressed at low levels in a host cell.

Similarly, a method for increasing the expression levels of the above identified Neisseria polynucleotide sequence(s) in a host cell further comprises the following steps:
(c) replacing codon 17 with a codon other than an ATC; and
(d) adding a 5′-ATG codon to the sequence, wherein codon 17 in step (c) is now codon 18.

In a preferred embodiment, codon 17 in step (c) is replaced with a TAC codon. In another embodiment, the method provides the following steps:
(e) infecting, transfecting or transforming a host cell with an expression vector comprising the polynucleotide of step (d),
(f) culturing the host cell under conditions suitable to produce the encoded protein or polypeptide, and
(g) recovering the protein or polypeptide from the culture.

The polynucleotide sequences of the invention can be obtained using standard molecular cloning techniques known in the art (e.g., PCR, etc.). The codon triplet sequence can be determined using well known techniques (e.g., DNA sequencing, in silico analysis). Methods for replacing codon 17, transforming and culturing a host cell and recovering the polypeptide are well known in the art, some of which have been described in Example 1 above.

In addition to Method I described above, the present invention further contemplates alternative steps of Method I (e.g., a variation of at least one of steps (a)-(g)). Thus, in certain embodiments, the invention is directed to alternatives of Method I for identifying Neisseria polynucleotide sequences encoding porin polypeptides which are expressed at low levels in a host cell and methods for increasing the expression levels of a Neisseria porin polypeptide in a host cell.

In another non-limiting example, a method for identifying “endogenous” Neisseria polynucleotide sequences encoding porin polypeptides expressed at low levels in a host cell comprises:

Method II:

(a) obtaining an “endogenous” Neisseria polynucleotide sequence;
(b) determining the 5′ signal sequence;
(c) hypothetically deleting the 5′ signal sequence; and
(d) determining the triplet sequence at codon 17 of the sequence in step (c), wherein an ATC at codon 17 indicates that the encoded porin protein or polypeptide is expressed at low levels in a host cell.

Similarly, a method for identifying “endogenous” Neisseria polynucleotide sequences encoding porin polypeptides expressed at low levels in a host cell and increasing the expression levels of said polypeptides in a host cell comprises:

Method III:

(a) obtaining an “endogenous” Neisseria polynucleotide sequence;
(b) determining the 5′ signal sequence;
(c) deleting the 5′ signal sequence;
(d) determining the triplet sequence at codon 17, wherein an ATC at codon 17 indicates that the encoded porin protein or polypeptide is expressed at low levels in a host cell; and
(e) replacing codon 17 with a codon other than an ATC.

In certain embodiments, the method further comprises:
(e) adding a 5′-ATG codon to the sequence, wherein codon 17 in step (e) is now codon 18.

In preferred embodiments, the method further comprises:
(g) infecting, transfecting or transforming a host cell with an expression vector comprising the polynucleotide of step (f),
(h) culturing the host cell under conditions suitable to produce the encoded protein or polypeptide, and
(i) recovering the protein or polypeptide from the culture.

In yet another non-limiting example, the invention describes methods for increasing Neisseria polypeptide expression levels in a host cell by utilizing an alternative Neisseria strain. Thus, in one embodiment, the invention provides a method for increasing the expression levels of a Neisseria porin polypeptide or protein in a host cell comprising:

Method IV:

(a) obtaining a “mature” Neisseria polynucleotide sequence;
(b) determining the triplet sequence at codon 17, wherein an ATC at codon 17 indicates that the encoded porin protein or polypeptide is expressed at low levels in a host cell; and
(c) selecting an alternative Neisseria strain wherein codon 17 of the mature alternative strain sequence is a codon other than an ATC.
   In certain embodiments, the method further comprises:
(d) adding a 5′-ATG codon to the alternative Neisseria sequence, wherein codon 17 in step (c) is now codon 18.
   In yet other embodiments, the method further comprises:
(e) infecting, transfecting or transforming a host cell with an expression vector comprising the polynucleotide of step (d),
(f) culturing the host cell under conditions suitable to produce the encoded protein or polypeptide, and
(g) recovering the protein or polypeptide from the culture.

In one preferred embodiment, the alternative strain in step (c) has a TAC at codon 17.

In another non-limiting example, the invention is directed to a method for increasing the expression levels of a Neisseria porin polypeptide or protein in a host cell comprising:

Method V:

(a) obtaining an endogenous Neisseria polynucleotide sequence;
(b) determining the 5′ signal sequence;
(c) hypothetically deleting the 5′ signal sequence;
(d) determining the triplet sequence at codon 17 of the sequence in step (c), wherein an ATC at codon 17 indicates that the encoded porin protein or polypeptide is expressed at low levels in a host cell; and
(e) selecting an alternative Neisseria strain wherein codon 17 of the mature alternative strain sequence is a codon other than an ATC.

In certain embodiments, the method further comprises:
(f) adding a 5′-ATG codon to the alternative Neisseria sequence, wherein codon 17 in step (e) is now codon 18.
   In another embodiment, the method further comprises:
(g) infecting, transfecting or transforming a host cell with an expression vector comprising the polynucleotide of step (f),
(h) culturing the host cell under conditions suitable to produce the encoded protein or polypeptide, and
(i) recovering the protein or polypeptide from the culture.
   In a preferred embodiment, the alternative strain in step (e) has a TAC at codon 17.

As shown in Table 6 below, alternative Neisseria meningitidis strains encoding the same porA serosubtype were identified in a database search (e.g., P1:5,10; P1:5b,10; P1:5b,10j and P1:5b,10h). Based on analysis of codon 18 of the mature +1 sequence (e.g., ATC vs. TAC or TTC or ATT) the majority of these strains encoding the P1:5,10 serosubtype are predicted to express the PorA polypeptide at a low levels in a host cell. For example, most derivative strains of Neisseria meningitidis encoding the P1:5,10 serosubtype have an ATC at codon 18 and do not express the PorA polypeptide. However, three P1:5,10 derivative strains were identified in the database search, wherein these derivative strains have a TAC at codon 18 and are predicted to express the PorA polypeptide at high levels in a host cell (e.g., ≧30% total cellular protein concentration).

TABLE 6

| N. meningitidis Strain | Serosubtype | Codon 18 |
|---|---|---|
| NMU 92931 | P1:5,10 | ATC |
| NMMC 129 | P1:5b,10 | TAC |
| NMMC 123 | P1:5b,10j | TAC |
| NMMC 117 | P1:5b,10h | TAC |

### Example 4

### Evaluating Immunopotency of the PorA Antigens

Table 7 lists the mouse immunogenicity data generated from both recombinant and native PorA protein used as the immunogen. The substantially pure serosubtype PorA proteins are isolated as described in Example 1 and mixed with 100 ug MPL and emulsified. Swiss Webster mice were injected intraperitoneally with approximately 5 μg of the PorA protein in the adjuvant mixture. Animals are reimmunized approximately 4 weeks after the initial immunization and bled two weeks following the final immunization. The whole cell ELISA (Abdillahi et al., 1987) and bactericidal (Mountzouros et al., 2000) assays were performed as described in the publications.

Table 7 summarizes the whole cell ELISA (WCE) and bactericidal (BC) assay data generated in Swiss Webster mice. The first columns show the serosubtypes designation of the PorA protein and the parental meningococcal strain from which it was derived. The WCE and BC assays are indicated in column 3. The 6 week antisera titers for both assays using the recombinant and native PorA proteins are indicated in columns 4 and 5. These data indicate that there is essentially no difference in reactogenicity or functional activity of the antisera raised against either the native or recombinant PorA immunogens, with or without the lie to Tyr amino acid change.

TABLE 7

| porA | Parental | | Recombinant | Native PorA |
|---|---|---|---|---|
| Serosubtype | Strain | Assay | PorA (5 ug) | (5 ug) |
| 7,16 | H44/76 | WCE | 657,000 | 1,249,000 |
| | | BC | >800 | >800 |
| 7b,4 | 880049 | WCE | 793,000 | 949,000 |
| | | BC | 50 | 50 |
| 22a,14 | 6557 | WCE | 686,000 | NA |
| | | BC | 400 | NA |
| 5a,2c | NMB | WCE | 1,114,000 | 1,657,000 |
| | | BC | >800 | >800 |
| 19,15 | H355 | WCE | 1,697,000 | 1,536,000 |
| | | BC | 200 | 200 |
| 5c,10 | 870227 | WCE | 764,000 | 416,000 |
| | | BC | 400 | 50 |

All vaccinations prepared with 100 ug MPL (adjuvant); WCE: Whole Cell ELISA; BC: Bactericidal Assay; PorA Ile to Tyr change in NMB (P1:5a,2c) and 870227 (P1:5c,10), data highlighted in bold text; NA = Not available.

### Example 5

### Generation of Polyclonal Antisera

A substantially pure serotype PorA protein is used as an immunogen to prepare anti-PorA antibodies. The PorA protein is isolated as described in Example 1 and mixed with incomplete Freund's adjuvant and emulsified. Rabbits are injected intramuscularly with approximately 50 μg of a PorA protein in the adjuvant mixture. Animals are reimmunized approximately 4 weeks and 8 weeks after the initial immunization and bled one week following the final immunization.

### Example 6

### In Vitro Opsonphagocytosis Analysis

An in vitro opsonic assay is conducted by incubating together a mixture of Neisseria meningitidis cells, heat inactivated human serum containing specific antibodies to the Neisseria strain, and an exogenous complement source. Opsonophagocytosis proceeds during incubation of freshly isolated human polymorphonuclear cells (PMN's) and the antibody/complement/Neisseria cell mixture. Bacterial cells that are coated with antibody and complement are killed upon opsonophagocytosis. Colony forming units (cfu) of surviving bacteria that escape from opsonophagocytosis are determined by plating the assay mixture. Titers are reported as the reciprocal of the highest dilution that gives ≧50% bacterial killing, as determined by comparison to assay controls. Specimens which demonstrate less than 50% killing at the lowest serum dilution tested (1:8), are reported as having an OPA titer of 4. The highest dilution tested is 1:2560. Samples with ≧50% killing at the highest dilution are repeated, beginning with a higher initial dilution.

The present method is a modification of Gray's method (Gray, 1990). The assay mixture is assembled in a 96-well microtiter tissue culture plate at room temperature. The assay mixture consists of 10 μL of test serum (a series of two-fold dilutions) heated to 56° C. for 30 minutes prior to testing, 10 μL of preclostral bovine serum (complement source) having no opsonic activity for the bacterial test strain, and 20 μL of buffer containing 2000 viable Neisseria meningitidis organisms. This mixture is incubated at 37° C. without CO₂ for 30 minutes with shaking. Next, 40 μL of human PMNs, freshly prepared from heparinized peripheral blood by dextran sedimentation and Percoll density centrifugation, suspended in buffer at a concentration of 1×10⁶/mL is added. The assay plate(s) are then incubated at 37° C. for an additional 90 minutes with vigorous shaking. Aliquots from each well are dispensed onto the upper ¼ of a 15×100 mm blood agar plate. The blood agar plate is tilted while pipetting to allow the liquid suspension to “run” down the plate. Plates are incubated overnight in 5% CO₂ at 37° C. The viable cfu are counted the following morning. Negative control wells, lacking bacterial cells, test serum, complement and/or phagocytes in appropriate combination are included in each assay. A test serum control, which contains test serum plus bacterial cells and heat inactivated complement, is included for each individual serum. This control can be used to assess whether the presence of antibiotics or other serum components are capable of killing the bacterial strain directly (i.e. in the absence of complement or PMN's). A human serum with known opsonic titer is used as a positive human serum control. The opsonic antibody titer for each unknown serum is calculated as the reciprocal of the initial dilution of serum giving 50% cfu reduction compared to the control without serum.

### Example 7

### Intranasal Immunization of Swiss Webster Mice Prior to Challenge

Six-week old, pathogen-free, outbred female Swiss Webster mice (Taconic Farms. Germantown, N.Y.) are housed in a filtered HEPA Rack systems under standard temperature, humidity, and lighting conditions. Mice (10/group) are anesthetized with 60 mg/Kg of ketamine HCl (Fort Dodge Laboratory, Ft. Dodge, Iowa) by i.p. injection, then immunized intranasally with a 10 ul volume on weeks 0, 2, and 3, with an appropriate amount of the protein to be tested. At each immunization the protein being tested is formulated with 0.1 μg of CT-E29H and slowly instilled into the nostrils of each mouse. Control groups receive the CT-E29H alone or are formulated with the Keyhole Limpet Hemocyanin (KLH) protein. Serum samples are collected at weeks 0 and 4 to determine antibody response.

### Example 8

### Mouse Intranasal Challenge Model

The Swiss Webster mice are challenged approximately at one week after the last immunization with approximately 1×10⁷ CFU's of piliated, infant rat passaged Neisseria meningitidis mixed with 80 μg of ferric dextran. The Neisseria meningitidis culture is grown overnight at 37° C. in 5% CO₂ on Theayer Martin improved agar plates. Neisseria meningitidis colonies are then inoculated into Modified Frantz Media at an OD₆₂₀ of 0.2. The culture is grown at 37° C. and an agitation of 70 rpm until the bacterial cells reached late-log phase. The cells are then keep at room temperature and used for the intranasal challenge. At 4 hours prior to challenge, 2 mg of ferric dextran is injected i.p. into each mouse. The bacterial suspension is inoculated into the nostrils of anesthetized mice (10 μl per nostril, 20 μl per mouse). The actual dose of bacterial administered is confirmed by plate count. Twenty four hours after challenge, mice are sacrificed, the noses removed, and homogenized in 3-ml sterile saline with a tissue homogenizer (Ultra-Turax T25, Janke & Kunkel Ika-Labortechnik, Staufen, Germany). The homogenate is 10-fold serially diluted in saline and plated on Thayer Martin plates. The plates are incubated overnight at 37° C. in 5% CO₂ and then the colonies are counted.

### REFERENCES

European Application No. EP 125023
European Application No. EP 171496
European Application No. EP 173494
European Application No. EP 184187
U.S. Pat. No. 4,196,265
U.S. Pat. No. 4,522,811
U.S. Pat. No. 4,554,101
U.S. Pat. No. 4,683,202
U.S. Pat. No. 4,816,567
U.S. Pat. No. 4,873,316
U.S. Pat. No. 5,223,409
U.S. Pat. No. 5,439,808
U.S. Pat. No. 6,013,267
U.S. Pat. No. 6,168,918
U.S. Pat. No. 6,201,103
U.S. Pat. No. 6,207,646
International Application No. WO 86/01533
International Application No. WO 90/02809
International Application No. WO 91/17271
International Application No. WO 92/01047
International Application No. WO 92/09690
International Application No. WO 92/15679
International Application No. WO 92/18619
International Application No. WO 92/20791
International Application No. WO 01/18434
Abdillahi and Poolman, FEMS Microbiol. Lett., 48:367-371, 1987.
Amann et al., Gene 69:301-315, 1988.
Artenstein et al., N Engl. J. Med. 282: 417420, 1970.
Banerji et al., Cell, 33:729-740; 1983.
Bjornson et al., Infect. Immun. 56:1602-1607, 1988.
Byrne and Ruddle, PNAS 86:5473-5477,1989.
Calame and Eaton, Adv. Immunol. 43:235-275, 1988.
Campes and Tilghman, Genes Dev. 3:537-546, 1989.
Carbonetti and Sparling, Proc. Natl. Acad. Sci. USA 84:9084-9088, 1987.
Carbonetti et al., Proc. Natl. Acad. Sci. USA 85:6841-6845, 1988.
Dawson et al, Pediatr. Infect Dis. J. 18:816-822, 1999.
Douglas et al., FEMS Microbiol. Lett. 12:305-309, 1981.
Evans et al, Biotechniques 19:130-135, 1995.
Feavers et al., Infect Immun. 60:3620-3629, 1992.
Finne et al., Lancet, p. 355, 1983.
Frasch et al, Clin. Invest. Med., 9, p. 101, 1986.
Frasch et al., Eur. J. Clin. Microbiol., 4, p.533, 1985(a).
Frasch et al, in Robbins, Bacterial Vaccines, p. 262, Praeger Publications, N.Y., 1987.
Frasch et al., Med. Trop., 43, p.177, 1982.
Frasch et al, Rev. Infect. Dis. 7:504-510, 1985(b).
Frasch, Clin. Microb., Rev. 2, p. S134, 1989.
Gold, “Evolution of meningococcal disease,” p. 69, Vedros N. A., CRC Press, 1987.
Goldschneider et al., J. Exp. Med., 129, p.1307, 1969.
Gotschlich et al., “Human immunity to the meningococcus IV. Immunogenicity of group A and group C meningococcal polysaccharides,” J. Exp Med. 129:

1367-1384, 1969(a).
Gotschlich et al., Proc. Natl. Acad. Sci. USA 84:8135-8139, 1987.
Gray, Conjugate Vaccines Supplement p694-697, 1990.
Guzman et al., J. Bact., 177: 4121-4130, 1995.
Harlow and Lane, “Antibodies: A Laboratory Manual,” Cold Spring Harbor Laboratory Press, Cold Spring Harbor, N.Y., 1988
Heasley et al., “Reconstitution and characterization of the N. gonorrhoeae outer membrane permeability barrier,” in Genetics and Immunobiology of Neisseria gonorrhoeae, Danielsson and Normark, eds., University of Umea, Umea, pp. 12-15, 1980.
Heckels, J. Gen. Microbiol 99:333-341, 1977.
Hernandez-Sanchez et al., EMBO Jour. 17(13):3758-65, 1998.
Hogan, “Manipulating the Mouse Embryo,” Cold Spring Harbor Laboratory Press, Cold Spring Harbor, N.Y., 1986.
James and Heckels, J. Immunol. Meth. 42:223-228, 1981.
Johansson et al., Protein Expr. Purif., 17:105-112, 1999.
Johnson et al., Endoc. Rev., 10:317-331, 1989.
Jones et al., Infect Imman., 30:773-780, 1980.
Judd, Anal. Biochem., 173:307-316, 1988.
Kaufman et al., EMBO J, 6:187-195, 1987.
Kessel and Gruss, Science, 249:3 74-3 79, 1990.
Knapp et al., “Overview of epidemiological and clinical applications of auxotype/serovar classification of Neisseria gonorrhoeae,” The Pathogenic Neisseriae, Schoolnik, G. K., ed., American Society for Microbiology, Washington, pp. 6-12, 1985.
Koebnik, Mol. Microbiol. 16(6):1269-70, 1995.
Koomey et al., Mol. Microbiol., 5:279, 1991.
Kyte and Doolittle, J. Mol. Biol., 157:105-132, 1982.
LaVallie et al., Bio/Technology, 11: 187-193, 1992.
Lennette, “General principles for laboratory diagnosis of viral, rickettsial, and chlamydial infections,” p. 17-18, diagnostic procedures for viral, rickettsial, and chlamydial infections, vol. 7th edition, 1995.
Li et al., Cell 69:915, 1992.
Lowry et al., J. Biol. Chem. 193:265-275, 1951.
Lynch et al., Biophys. J. 41:62, p3, 1984(a).
Lynch et al., Biophys. J. 45:104-107,1984(b).
Mauro et al., Proc. Natl. Acad. Sci. USA 85:1071-1075, 1988.
McGuinness et al., J. Exp. Med. 171:1871-1882, 1990.
Moreno et al., Infec. Immun., 47, p. 527, 1985.
Morin et al., Nucleic Acids Res., 21:2157-2163, 1993.
Mountzouros and Howell, J. Clin. Microbiol., 38:2878-2884, 2000.
Murakmi et al., Infect. Immun. 57:2318-2323, 1989.
Myers et al., Nature 313:495, 1985(a).
Myers et al., Science 230:1242, 1985(b).
O'Gon-nan et al., Science 251:1351-1355, 1991.
Orita et al., PNAS 86:2766, 1989.
Parkhill et al., Nature 404(6777):502-6, 2000.
Pinkert et al. Genes Dev. 1:268-277, 1987.
Pizza et al., Science 287(5459):1816-20, 2000.
Pugsley, “The complete general secretory pathway in gram-negative bacteria,” Microbiol. Rev. 57:50-108, 1993.
Queen and Baltimore, Cell 33:741-748, 1983.
Rahman et al., Journal of Neuroscience 19:2016-2026, 1999.
Reingold et al., Lancet, p. 114, 1985.
Riedo et al., Pediatr. Infect. Dis. J. 14:643-657, 1995.
Rose et al., “Methods in Yeast Genetics: A Laboratory Course Manual.” Cold Spring Harbor Press, Cold Spring Harbor, N.Y. (1990).
Saleeba et al., Meth. Enzymol. 217:286-295, 1992.
Salzberg et al., Nuc. Acids Res. 26(2):544-8, 1998.
Sambrook et al., “Molecular Cloning: A Laboratory Manual” 2nd, ed, Cold Spring Harbor Laboratory, Cold Spring Harbor Laboratory Press, Cold Spring Harbor, N.Y., 1989.
Sanger, PNAS 74:5463, 1977.
Schultz et al., Gene 54:113-123, 1987.
Schwartz et al., Clin. Microbiol. Rev., 2, p. S118, 1989.
Seed, Nature 329:840, 1987.
Seki and Arai, Neurosci. Res. 17:265-290, 1993.
Sierra et al., NIPH Annals, 14, p.195, 1991.
Simon et al., Science, 252:802-8, 1991.
Skevakis et al., J. Infect Dis., 149, p. 387, 1984.
Smith and Johnson, Gene 67:31-40, 1988.
Smith et al., Mol. Cell Biol. 3:2156-2165, 1983.
Songyang, et al., Cell 72:767-778, 1993.
Studier et al. “Gene Expression Technology” Methods in Enzymology 185, 60-89, 1990.
Tebbey et al., Vaccine 18(24):2723-2734, 2000.
Thomas and Capecchi, Cell 51:503, 1987.
Ward et al., FEMS Microbiol. Lett. 73:283-289, 1992.
Wetzler et al., J. Ex. Med. 168:1883-1897, 1988.
Wilmut et al., Nature 385:810-813, 1997.
Wilson et al., Cell 37:767, 1984.
Winoto and Baltimore. EMBO J 8:729-733, 1989.
Wolff and Stern, FEMS Microbiol Lett. 83:179-186, 1991.
Wyle et al., J. Infect Dis. 126: 514-521, 1972.
Young et al., Proc. Natl. Acad. Sci. USA 80:3831-3835, 1983.
Young et al., Proc. Natl. Acad. Sci. USA 83:150-154, 1986.
Zagursky et al., Gene 27:183-191, 1984.
Zagursky and Russell, “Bioinfomatics: Use in bacterial vaccine research,” Biotechniques, 31:636-659, 2001.
Zervos et al., Cell 72:223-232, 1993. Zollinger et al., J. Clin. Invest., 63, p. 836, 1979.
Zollinger et al., NIPH Annals, 14, p. 211, 1991.
Zollinger, in Woodrow and Levine, “New generation vaccines,” p. 325, Marcel Dekker Inc. N.Y., 1990.

What is claimed is:

1. A method for increasing the expression levels of a Neisseria PorA protein or polypeptide in a host cell comprising the steps of:
(a) infecting, transfecting or transforming a host cell with an expression vector comprising a polynucleotide comprising a nucleotide sequence of SEQ ID NO:1, SEQ ID NO:3, SEQ ID NO:13, SEQ ID NO:15 or SEQ ID NO:24, wherein codon 18 is a codon other than an ATC;
(b) culturing the host cell under conditions suitable to produce the protein or polypeptide encoded by the polynucleotide of step (a); and
(c) recovering the protein or polypeptide from the culture.

2. The method of claim 1, wherein the polynucleotide comprising the nucleotide sequence of SEQ ID NO:1 encodes a protein or polypeptide comprising an amino acid sequence of SEQ ID NO:2, wherein the amino acid at residue 18 is an amino acid other than an ATC encoded isoleucine residue.

3. The method of claim 2, wherein the polynucleotide encoding the PorA protein or polypeptide is isolated from Neisseria meningitidis.

4. The method of claim 1, wherein the polynucleotide comprising the nucleotide sequence of SEQ ID NO:3 encodes a protein or polypeptide comprising an amino acid sequence of SEQ ID NO:4, wherein the amino acid at residue 18 is an amino acid other than an ATC encoded isoleucine residue.

5. The method of claim 4, wherein the polynucleotide encoding the PorA protein or polypeptide is isolated from Neisseria meningitidis.

6. The method of claim 1, wherein the polynucleotide comprising the nucleotide sequence of SEQ ID NO:13 encodes a protein or polypeptide comprising an amino acid sequence of SEQ ID NO:14, wherein the amino acid at residue 18 is an amino acid other than an ATC encoded isoleucine residue.

7. The method of claim 6, wherein the polynucleotide encoding the PorA protein or polypeptide is isolated from Neisseria meningitidis.

8. The method of claim 1, wherein the polynucleotide comprising the nucleotide sequence of SEQ ID NO:15 encodes a protein or polypeptide comprising an amino acid sequence of SEQ ID NO:16, wherein the amino acid at residue 18 is an amino acid other than an ATC encoded isoleucine residue.

9. The method of claim 8, wherein the polynucleotide encoding the PorA protein or polypeptide is isolated from Neisseria meningitidis.

10. The method of claim 1, wherein the polynucleotide comprising the nucleotide sequence of SEQ ID NO:24 encodes a protein or polypeptide comprising an amino acid sequence of SEQ ID NO:25, wherein the amino acid at residue 18 is an amino acid other than an ATC encoded isoleucine residue.

11. The method of claims 10, wherein the polynucleotide encoding the PorA protein or polypeptide is isolated from Neisseria meningitidis.

12. The method of claim 1, wherein codon 18 is a TAC codon.

13. The method of claim 1, wherein the polynucleotide is operatively linked to one or more gene expression regulatory elements.

14. The method of claim 13, wherein one of the regulatory elements is a promoter.

15. The method of claim 1, wherein the vector is a plasmid.

16. The method of claim 15, wherein the plasmid is pET9a.

17. The method of claim 1, wherein the host cell is a bacterial cell.

18. The method of claim 17, wherein the host cell is E. coli.

19. The method of claim 18, wherein the E. coli is a DE3 lysogenic strain.

20. The method of claim 19, wherein the strain is selected from the group consisting of BLR(DE3)pLysS, BL21(DE3)pLysS, HMS174(DE3)pLysE and NovaBlue(DE3).

21. The method of claim 1, wherein the protein or polypeptide expressed is at least about 30% of the total cellular protein concentration.

22. The method of claim 1, wherein the protein or polypeptide expressed is at least about 50% of the total cellular protein concentration.

23. The method of claim 1, wherein the protein or polypeptide expressed is at least about 75% of the total cellular protein concentration.

24. An isolated PorA protein or polypeptide produced according to the method of claim 1.

25. An isolated Neisseria meningitidis polynucleotide comprising a nucleotide sequence of SEQ ID NO:1, SEQ ID NO:3, SEQ ID NO:13, SEQ ID NO:15 or SEQ ID NO:24, wherein codon 18 is a codon other than an ATC codon.

26. The polynucleotide of claim 25, wherein codon 18 is a TAC codon.

27. An isolated Neisseria meningitidis PorA polypeptide or protein comprising an amino acid sequence of SEQ ID NO:2, SEQ ID NO:4, SEQ ID NO:14, SEQ ID NO:16 or SEQ ID NO:25, wherein the amino acid at residue 18 is an amino acid other than an ATC encoded isoleucine.

28. The polypeptide or protein of claim 27, wherein the amino acid at residue 18 is tyrosine.

29. A recombinant expression vector comprising a polynucleotide having a nucleotide sequence of SEQ ID NO:1, SEQ ID NO:3, SEQ ID NO:13, SEQ ID NO:15 or SEQ ID NO:24, wherein codon 18 is a codon other than an ATC codon.

30. The vector of claim 29, wherein codon 18 is a TAC codon.

31. The vector of claim 30, wherein the polynucleotide is selected from the group consisting of DNA, cDNA, genomic DNA, RNA and mRNA.

32. The vector of claim 31, wherein the vector is plasmid DNA.

33. The vector of claim 32, wherein the polynucleotide is operatively linked to one or more gene expression regulatory elements.

34. A genetically engineered host cell transfected, transformed or infected with the vector of claim 29.

35. The host cell of claim 34, wherein the cell is a bacterial cell.

36. The host cell of claim 35, wherein the bacterial cell is E. coli.

37. The host cell of claim 36, wherein the E. coli is a DE3 lysogenic strain.

38. The host cell of claim 37, wherein the strain selected from the group consisting of BLR(DE3)pLysS, BL21(DE3)pLysS, HMS174(DE3)pLysE and NovaBlue(DE3).

39. The host cell of claim 34, wherein the polynucleotide is expressed to produce the encoded polypeptide or protein.

40. An immunogenic composition comprising a Neisseria meningitidis PorA polypeptide or protein having an amino acid sequence of SEQ ID NO:2, SEQ ID NO:4, SEQ ID NO:14, SEQ ID NO:16 or SEQ ID NO:25, wherein the amino acid at residue 18 is an amino acid other than an ATC encoded isoleucine.

41. The immunogenic composition of claim 40, wherein the amino acid at residue 18 is tyrosine.

42. The immunogenic composition of claim 41, further comprising one or more PorA polypeptides or proteins selected from the group consisting of SEQ ID NO:6, SEQ ID NO:8, SEQ ID NO:10, SEQ ID NO:12, SEQ ID NO:18 and SEQ ID NO:20.

43. The immunogenic composition of claim 42, further comprising one or more adjuvants.

44. A method for identifying Neisseria polynucleotide sequences encoding PorA proteins or polypeptides which are expressed at low levels in a host cell, the method comprising:
(a) obtaining a mature Neisseria polynucleotide sequence; and
(b) determining the triplet sequence at codon 17, wherein an ATC at codon 17 indicates that the encoded PorA protein or polypeptide is expressed at low levels in a host cell.

45. An isolated polynucleotide identified according to the method of claim 44.

46. A recombinant expression vector comprising the polynucleotide of claim 45.

47. A genetically engineered host cell transfected, transformed or infected with the vector of claim 46.

48. A method for identifying Neisseria polynucleotide sequences encoding PorA proteins or polypeptides which are expressed at low levels in a host cell, the method comprising:
(a) obtaining an endogenous Neisseria polynucleotide sequence;
(b) determining the 5′ signal sequence;
(c) hypothetically deleting the 5′ signal sequence; and
(d) determining the triplet sequence at codon 17 of the sequence in step (c), wherein an ATC at codon 17 indicates that the encoded PorA protein or polypeptide is expressed at low levels in a host cell.

49. An isolated polynucleotide identified according to the method of claim 48.

50. A recombinant expression vector comprising the polynucleotide of claim 49.

51. A genetically engineered host cell transfected, transformed or infected with the vector of claim 50.

52. A method for increasing the expression levels of a Neisseria PorA polypeptide or protein in a host cell, the method comprising:
(a) obtaining a mature Neisseria polynucleotide sequence;
(b) determining the triplet sequence at codon 17, wherein an ATC at codon 17 indicates that the encoded PorA protein or polypeptide is expressed at low levels in a host cell; and
(c) replacing codon 17 with a codon other than an ATC.

53. The method of claim 52, further comprising step (d), adding a 5′-ATG codon to the sequence, wherein codon 17 in step (c) is now codon 18.

54. An isolated polynucleotide produced according to the method of claim 53.

55. The method of claim 53, further comprising the steps of:
(e) infecting, transfecting or transforming a host cell with an expression vector comprising the polynucleotide of step (d),
(f) culturing the host cell under conditions suitable to produce the encoded protein or polypeptide, and
(g) recovering the protein or polypeptide from the culture.

56. The method of claim 52, wherein replacing codon 17 in step (c) is a TAC codon.

57. An isolated polypeptide produced according to the method of claim 55.

58. An immunogenic composition comprising the polypeptide of claim 57.

59. A recombinant expression vector comprising the polynucleotide of claim 54.

60. A genetically engineered host cell transfected, transformed or infected with the vector of claim 59.

61. A method for increasing the expression levels of a Neisseria PorA polypeptide or protein in a host cell, the method comprising:
(a) obtaining an endogenous Neisseria polynucleotide sequence;
(b) determining the 5′ signal sequence;
(c) deleting the 5′ signal sequence;
(d) determining the triplet sequence at codon 17, wherein an ATC at codon 17 indicates that the encoded protein or polypeptide is expressed at low levels in a host cell; and
(e) replacing codon 17 with a codon other than an ATC.

62. The method of claim 61, further comprising step (f), adding a 5′-ATG codon to the sequence, wherein codon 17 in step (e) is now codon 18.

63. An isolated polynucleotide produced according to the method of claim 62.

64. The method of claim 62, further comprising the steps of:
(g) infecting, transfecting or transforming a host cell with an expression vector comprising the polynucleotide of step (f),
(h) culturing the host cell under conditions suitable to produce the encoded protein or polypeptide, and
(i) recovering the protein or polypeptide from the culture.

65. The method of claim 61, wherein replacing codon 17 in step (c) is a TAC codon.

66. An isolated polypeptide produced according to the method of claim 64.

67. An immunogenic composition comprising the polypeptide of claim 66.

68. A recombinant expression vector comprising the polynucleotide of claim 63.

69. A genetically engineered host cell transfected, transformed or infected with the vector of claim 68.

70. A method for increasing the expression levels of a Neisseria PorA polypeptide or protein in a host cell, the method comprising:
(a) obtaining a mature Neisseria polynucleotide sequence;
(b) determining the triplet sequence at codon 17, wherein an ATC at codon 17 indicates that the encoded protein or polypeptide is expressed at low levels in a host cell; and
(c) selecting an alternative Neisseria strain wherein codon 17 of the mature alternative strain sequence is a codon other than an ATC.

71. The method of claim 70, further comprising step (d), adding a 5′-ATG codon to the alternative Neisseria sequence, wherein codon 17 in step (c) is now codon 18.

72. An isolated polynucleotide produced according to the method of claim 71.

73. The method of claim 71, further comprising the steps of:
(e) infecting, transfecting or transforming a host cell with an expression vector comprising the polynucleotide of step (d),
(f) culturing the host cell under conditions suitable to produce the encoded protein or polypeptide, and
(g) recovering the protein or polypeptide from the culture.

74. The method of claim 70, wherein the alternative strain in step (c) has a TAC at codon 17.

75. An isolated polypeptide produced according to the method of claim 73.

76. An immunogenic composition comprising the polypeptide of claim 75.

77. A recombinant expression vector comprising the polynucleotide of claim 72.

78. A genetically engineered host cell transfected, transformed or infected with the vector of claim 77.

79. A method for increasing the expression levels of a Neisseria PorA polypeptide or protein in a host cell, the method comprising:
(a) obtaining an endogenous Neisseria polynucleotide sequence;
(b) determining the 5′ signal sequence;
(c) hypothetically deleting the 5′ signal sequence;
(d) determining the triplet sequence at codon 17 of the sequence in step (c), wherein an ATC at codon 17 indicates that the encoded protein or polypeptide is expressed at low levels in a host cell; and
(e) selecting an alternative Neisseria strain wherein codon 17 of the mature alternative strain sequence is a codon other than an ATC.

80. The method of claim 79, further comprising step (f), adding a 5′-ATG codon to the alternative Neisseria sequence, wherein codon 17 in step (e) is now codon 18.

81. An isolated polynucleotide produced according to the method of claim 80.

82. The method of claim 80, further comprising the steps of:
(g) infecting, transfecting or transforming a host cell with an expression vector comprising the polynucleotide of step (f),
(h) culturing the host cell under conditions suitable to produce the encoded protein or polypeptide, and
(i) recovering the protein or polypeptide from the culture.

83. The method of claim 80, wherein the alternative strain in step (f) has a TAC at codon 17.

84. An isolated polypeptide produced according to the method of claim 82.

85. An immunogenic composition comprising the polypeptide of claim 84.

86. A recombinant expression vector comprising the polynucleotide of claim 81.

87. A genetically engineered host cell transfected, transformed or infected with the vector of claim 86.

88. A method of immunizing against Neisseria comprising administering to a host an immunizing amount of an immunogenic composition comprising a polypeptide having an amino acid sequence of SEQ ID NO:2, or a fragment thereof and a pharmaceutically acceptable carrier, wherein the amino acid at residue 18 is an amino acid other than an ATC encoded isoleucine.

89. The method of claim 88, wherein the amino acid at residue 18 is tyrosine.

90. A method of immunizing against Neisseria comprising administering to a host an immunizing amount of an immunogenic composition comprising a polypeptide having an amino acid sequence of SEQ ID NO:4, or a fragment thereof and a pharmaceutically acceptable carrier, wherein the amino acid at residue 18 is an amino acid other than an ATC encoded isoleucine.

91. The method of claim 90, wherein the amino acid at residue 18 is tyrosine.

92. A method of immunizing against Neisseria comprising administering to a host an immunizing amount of an immunogenic composition comprising a polypeptide having an amino acid sequence of SEQ ID NO:14, or a fragment thereof and a pharmaceutically acceptable carrier, wherein the amino acid at residue 18 is an amino acid other than an ATC encoded isoleucine.

93. The method of claim 92, wherein the amino acid at residue 18 is tyrosine.

94. A method of immunizing against Neisseria comprising administering to a host an immunizing amount of an immunogenic composition comprising a polypeptide having an amino acid sequence of SEQ ID NO:16, or a fragment thereof and a pharmaceutically acceptable carrier, wherein the amino acid at residue 18 is an amino acid other than an ATC encoded isoleucine.

95. The method of claim 94, wherein the amino acid at residue 18 is tyrosine.

96. A method of immunizing against Neisseria comprising administering to a host an immunizing amount of an immunogenic composition comprising a polypeptide having an amino acid sequence of SEQ ID NO:25, or a fragment thereof and a pharmaceutically acceptable carrier, wherein the amino acid at residue 18 is an amino acid other than an ATC encoded isoleucine.

97. The method of claim 72, wherein the amino acid at residue 18 is tyrosine.

99. A method of immunizing against Neisseria comprising administering to a host an immunizing amount of an immunogenic composition comprising a polypeptide having an amino acid sequence of SEQ ID NO:2 or a fragment thereof, a polypeptide having an amino acid sequence of SEQ ID NO:4 or a fragment thereof, a polypeptide having an amino acid sequence of SEQ ID NO:14 or a fragment thereof, a polypeptide having an amino acid sequence of SEQ ID NO:16 or a fragment thereof, a polypeptide having an amino acid sequence of SEQ ID NO:25 or a fragment thereof and a pharmaceutically acceptable carrier, wherein the amino acid at residue 18 of SEQ ID NO:2, SEQ ID NO:4, SEQ ID NO:14, SEQ ID NO:16 and SEQ ID NO:25 is an amino acid other than an ATC encoded isoleucine.

100. The method of claim 99, wherein the amino acid at residue 18 is tyrosine.

101. The method according to any one of claims 44-53b, further comprising an adjuvant.

102. The method according to any one of claims 44-53b, further comprising one or more PorA polypeptides or proteins selected from the group consisting of SEQ ID NO:6, SEQ ID NO:8, SEQ ID NO:10, SEQ ID NO:12, SEQ ID NO:14, SEQ ID NO:16, SEQ ID NO:18 and SEQ ID NO:20.

103. An immunogenic composition according to any one of claims 42, 67,76 or 85, further comprising one or more ORF2086 protein antigens comprising an amino acid sequence of SEQ ID NO:26 through SEQ ID NO:83.
